(19) United States
(12) Patent Application Publication
(10) Pub. No.: US 2024/0384323 A1
(43) Pub. Date: Nov. 21, 2024

(54) METHOD FOR DETERMINING PROTEASE ACTIVITY IN A BIOLOGICAL SAMPLE

(71) Applicant: CytomX Therapeutics, Inc., South San Francisco, CA (US)

(72) Inventors: Olga VASILJEVA, Highlands Ranch, CO (US); Bruce HOWNG, Piedmont, CA (US); Michael B. WINTER, San Francisco, CA (US); Alexey Yevgenyevich BEREZHNOY, Burlingame, CA (US); Carol LEPAGE, Concord, CA (US)

(21) Appl. No.: 18/687,182

(22) PCT Filed: Aug. 30, 2022
(86) PCT No.: PCT/US2022/075700

§ 371 Date: Feb. 27, 2024

Related U.S. Application Data
(60) Provisional application No. 63/238,761, filed on Aug. 30, 2021.

(51) Int. Cl. C12Q 1/37

(52) U.S. Cl. CPC C12Q 1/37; G01N 2333/96433

(57) ABSTRACT

A method of determining protease activity in a biological sample comprising: (a) contacting the biological sample with a solution comprising a QZ probe to form a mixture; (b) incubating the mixture, thereby forming an incubated mixture comprising an incubated liquid; and (c) measuring the quantity of one or more analytes in a sample of the incubated liquid to determine the level of protease activity in the biological sample.

### CROSS-REFERENCE TO RELATED APPLICATION

This application claims the benefit of provisional application U.S. Ser. No. 63/238,761, filed Aug. 30, 2021, pursuant to 35 U.S.C. § 119(e), which is incorporated herein by reference in its entirety.

### REFERENCE TO SEQUENCE LISTING

The “Sequence Listing” submitted electronically concurrently herewith pursuant 37 C.F.R. § 1.821 in computer readable form (CRF) via EFS-Web as file name “4862_142_PCT_Sequence_Listing_Final.XML” is incorporated herein by reference. The electronic copy of the Sequence Listing was created on Aug. 30, 2022, and the size on disk is 151,552 bytes.

### FIELD OF THE INVENTION

The present disclosure relates to the field of biotechnology, and more specifically, to a method for assessing protease activity in a biological sample and applications thereof.

### BACKGROUND

Proteases, or proteolytic enzymes, catalyze the breakdown of proteins by hydrolysis of peptide bonds. More than 500 proteases (˜2% of the genome) have been identified using bioinformatic analysis of murine and human genomes and can be categorized in five distinct classes based on their catalytic mechanisms: serine, cysteine, aspartic, metalloproteases, and threonine proteases. Proteases are involved in the control of a multitude of key physiological processes, such as hemostasis, immunity, fertility, cell survival, proliferation and differentiation, and apoptosis.

Normally, protease activity is tightly regulated through multiple redundant mechanisms, including gene expression, zymogen activation, endogenous inhibitors, subcellular localization, and post-translational modifications. Protease dysregulation has been identified in a wide range of pathologies, including cardiovascular, neurodegenerative and inflammatory diseases, infection, and cancer. Notably, dysregulated proteolysis is central to carcinogenesis by playing key roles in tumor progression-associated processes, including growth, invasion, and metastasis. Due to their involvement in multiple pathologies, proteases represent attractive biomarkers or drug targets in wide-ranging therapeutic areas, including cancer.

Protease expression levels can be measured using mRNA quantification, proteomics, or by immunoassays, such as immunohistochemistry (IHC) or enzyme-linked immunosorbent assays (ELISAs). However, because proteases are regulated by multiple post-translational mechanisms, mRNA expression and immunoassays are not necessarily predictive of protease activity levels. For example, mRNA expression and immunoassays are unable to distinguish between active and inactive zymogen forms of proteases or those complexed with endogenous protease inhibitors.

In contrast, standard zymography, which relies upon visualization of enzymatic substrate conversion, enables direct measurement of protease activity through detection of cleavage product formation, or alternatively, substrate depletion. The combined use of molecular weight separation and zymography in the standard in-gel zymography approach provides qualitative, as well as quantitative, information and allows for differentiation of intact, activated, and complexed proteases. However, the tissue homogenization process often utilized for in-gel zymography may allow aberrant ex vivo proteolysis of substrates, which would impact the assay outcome. In addition, proteases can be denatured during the electrophoresis process. While in situ zymography of tissue sections obviates these risks, these approaches generally rely on broad-spectrum, dye-quenched protein substrates such as DQ-gelatin, DQ-collagen I, and DQ-collagen IV. Several types of zymography approaches therefore exist, but there remains an outstanding need for robust, reliable, and widely applicable techniques for estimating physiological and pathological protease activity.

### SUMMARY OF THE INVENTION

In one aspect, the present disclosure provides a method of determining the level of protease activity in a biological sample, the method comprising:
(a) contacting the biological sample with a solution comprising a quantitative zymography (QZ) probe to form a mixture, wherein the QZ probe comprises at least one bipartite polypeptide having a component A, a CL, and a component B in a structural arrangement of, from N-terminus to C-terminus,

A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide comprising the component A or a portion thereof, and a cleaved polypeptide comprising the component B or a portion thereof,
(b) incubating the mixture, thereby forming an incubated mixture comprising an incubated liquid; and
(c) measuring the quantity of one or more analytes in a sample of the incubated liquid to determine the level of protease activity in the biological sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof. In some embodiments, the biological sample is a cell, a cell culture, or a tissue sample. In some embodiments, the biological sample is an organoid.

In a specific aspect, the present disclosure provides a method of determining the level of protease activity in a biological sample, the method comprising:
(a) contacting a biological sample with a solution comprising a QZ probe to form a mixture, wherein the biological sample is a tissue sample, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising cleaved polypeptide that comprises the component A or a portion thereof and cleaved polypeptide comprising the component B or a portion thereof,
(b) incubating the mixture, thereby forming an incubated mixture comprising the tissue sample and an incubated liquid; and
(c) measuring the quantity of one or more analytes in a sample of the incubated liquid to determine the level of protease activity in the tissue sample, wherein the analyte is selected from the group consisting of cleaved polypeptide comprising the component A or portion thereof, cleaved polypeptide comprising the component B or portion thereof, an uncleaved bipartite polypeptide, and any combination of two or more thereof.

In a further specific aspect, the present disclosure provides a method of determining the level of protease activity in a biological sample comprising a liquid, the method comprising:
(a) contacting a biological sample with a QZ probe to form a mixture, wherein the biological sample comprises a liquid, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising cleaved polypeptide comprising one or more cleaved polypeptide species comprising the component A or a portion thereof and cleaved polypeptide comprising the component B or a portion thereof,
(b) incubating the mixture thereby forming an incubated mixture comprising an incubated liquid; and
(c) measuring the quantity of one or more analytes in a sample of incubated liquid to determine the level of protease activity in the biological sample, wherein the analyte is selected from the group consisting of cleaved polypeptide comprising the component A or portion thereof, cleaved polypeptide comprising the component B or portion thereof, an uncleaved bipartite polypeptide, and any combination of two or more thereof.

In some embodiments, the biological sample is selected from the group consisting of a cell culture supernatant, a sample of cells, an organoid, a tissue sample, a cell lysate supernatant, an organoid culture supernatant, blood, bile, bone marrow aspirate, breast milk, cerebrospinal fluid, plasma, saliva, serum, sputum, synovial fluid, and urine. In some embodiments, the biological sample is plasma.

In some embodiments, the bipartite polypeptide comprises the structure of, from N-terminus to C-terminus, B-CL-A. In some embodiments, the QZ probe comprises a polypeptide complex comprising one or more further polypeptides. In some embodiments, the QZ probe comprises an antibody, wherein at least one of components A and B of the bipartite polypeptide comprises an antibody domain selected from the group consisting of a light chain variable domain, a heavy chain variable domain, and a combination thereof.

In some embodiments, the QZ probe comprises an antibody comprising a first polypeptide, a second polypeptide, a third polypeptide, and a fourth polypeptide, wherein: (1) the first polypeptide comprises a bipartite polypeptide comprising a first component A that comprises a first light chain variable domain; (2) the second polypeptide comprises a second component A comprising a second light chain variable domain; (3) the third polypeptide comprises a first heavy chain variable domain; and (4) the fourth polypeptide comprises a second heavy chain variable domain.

In some embodiments, the QZ probe comprises an antibody comprising a first polypeptide, a second polypeptide, a third polypeptide, and a fourth polypeptide, wherein: (1) the first polypeptide comprises a bipartite polypeptide comprising a first component A that comprises a first heavy chain variable domain, (2) the second polypeptide comprises a second component A comprising a second heavy chain variable domain; (3) the third polypeptide comprises a first light chain variable domain; and (4) the fourth polypeptide comprises a second light chain variable domain.

In some embodiments, each component B comprises a polypeptide having at least 3 amino acid residues. In some embodiments, the QZ probe comprises an activatable antibody and each component B comprises a masking moiety.

In some embodiments, the QZ probe comprises an activatable antibody comprising a first, a second, a third, and a fourth polypeptide, wherein: the first polypeptide is a first light chain comprising a first bipartite polypeptide, wherein the component A comprises a first light chain variable domain, and a light chain constant domain, and the component B comprises a first masking moiety; the second polypeptide is a second light chain comprising a second bipartite polypeptide, wherein the component A comprises a second light chain variable domain, and a light chain constant domain, and the component B comprises a second masking moiety; the third polypeptide is a first heavy chain comprising a first heavy chain variable domain, and a CH1, a CH2, a CH3, a hinge region, and an Fc domain; and the fourth polypeptide is a second heavy chain comprising a second heavy chain variable domain, and a CH1, a CH2, a CH3, a hinge region, and an Fc domain.

In some embodiments, the QZ probe comprises an activatable antibody comprising a first, a second, a third, and a fourth polypeptide, wherein: the first polypeptide is a first light chain comprising a first light chain variable domain, and a light chain constant domain; the second polypeptide is a second light chain comprising a second light chain variable domain and a light chain constant domain; the third polypeptide is a first heavy chain comprising a first bipartite polypeptide, wherein the component A comprises a first heavy chain variable domain and a CH1, a CH2, a CH3, a hinge region, and an Fc domain, and the component B comprises a first masking moiety; and the fourth polypeptide is a second heavy chain comprising a bipartite polypeptide, wherein the component A comprises a second heavy chain variable domain, and a CH1, a CH2, a CH3, a hinge region, and an Fc domain, and component B comprises a second masking moiety.

In some embodiments, the QZ probe comprises a pseudo-antibody, wherein one of the component A and the component B comprises a pseudo-antibody variable domain selected from the group consisting of a pseudo-light chain variable domain and a pseudo-heavy chain variable domain, and a combination thereof. In some embodiments, the QZ probe comprises a pseudo-antibody comprising the bipartite polypeptide and a second polypeptide, wherein either (i) the component A comprises a pseudo-light chain variable domain and the second polypeptide comprises a domain selected from the group consisting of a heavy chain variable domain and a pseudo-heavy chain variable domain; or (ii) the component A comprises a pseudo-heavy chain variable domain and the second polypeptide comprises a domain selected from the group consisting of a light chain variable domain and a pseudo-light chain variable domain.

In some embodiments, the QZ probe comprises a pseudo-antibody comprising the bipartite polypeptide and a second polypeptide, wherein either (i) the component A comprises a light chain variable domain and the second polypeptide comprises a pseudo-heavy chain domain; or (ii) the component A comprises a heavy chain variable domain and the second polypeptide comprises a pseudo-light chain variable domain.

In some embodiments, the QZ probe comprises a pseudo-antibody comprising a first polypeptide, a second polypeptide, a third polypeptide, and a fourth polypeptide, wherein: (1) the first polypeptide is a light chain comprising a bipartite polypeptide, wherein the component A comprises a domain selected from the group consisting of a first light chain variable domain and a first pseudo-light chain variable domain; (2) the second polypeptide is a light chain comprising a bipartite polypeptide, wherein the component A comprises a domain selected from the group consisting of a second light chain variable domain and a second pseudo-light chain variable domain; (3) the third polypeptide is a heavy chain comprising a first pseudo-heavy chain variable domain; and (4) the fourth polypeptide is a heavy chain comprising a second pseudo-heavy chain variable domain.

In some embodiments, the QZ probe comprises a pseudo-antibody comprising a first polypeptide, a second polypeptide, a third polypeptide, and a fourth polypeptide, wherein (1) the first polypeptide is a light chain comprising a first bipartite polypeptide, wherein the component A comprises a first pseudo-light chain variable domain; (2) the second polypeptide is a light chain comprising a second bipartite polypeptide, wherein the component A comprises a second pseudo-light chain variable domain; (3) the third polypeptide is a heavy chain comprising a domain selected from the group consisting of a first heavy chain variable domain and a first pseudo-heavy chain variable domain; and (4) the fourth polypeptide is a heavy chain comprising a domain selected from the group consisting of a second heavy chain variable domain and a second pseudo-heavy chain variable domain.

In some embodiments, each component B is a polypeptide comprising at least about 3 amino acid residues. In some embodiments, the pseudo-antibody is a variant of a parental antibody and wherein each component B comprises a parental masking moiety.

In some embodiments, the QZ probe comprises an activatable pseudo-antibody comprising a first polypeptide, a second polypeptide, a third polypeptide, and a fourth polypeptide, wherein: the first polypeptide is a first light chain comprising a first bipartite polypeptide, wherein the component A comprises (i) a first domain selected from the group consisting of first pseudo-light chain variable domain and a first light chain variable domain, and (ii) a light chain constant domain, and the component B comprises a first parental masking moiety; the second polypeptide is a second light chain comprising a second bipartite polypeptide, wherein the component A comprises (i) a second domain selected from the group consisting of a second pseudo-light chain variable domain and a second light chain variable domain, and (ii) a light chain constant domain, and the component B comprises a second parental masking moiety; the third polypeptide is a heavy chain comprising (i) a third domain selected from the group consisting of a first pseudo-heavy chain domain and a first heavy chain variable domain, and (ii) a CH1, a CH2, a CH3, a hinge region, and an Fc domain; and the fourth polypeptide is a heavy chain comprising (i) fourth domain selected from the group consisting of a second pseudo-heavy chain domain and a second heavy chain variable domain, and (ii) a CH1, a CH2, a CH3, a hinge region, and an Fc domain, with the proviso that at least one of the first domain, the second domain, the third domain, and the fourth domain is a pseudo-variable domain.

In some embodiments, the QZ comprises an activatable pseudo-antibody comprising a first polypeptide, a second polypeptide, a third polypeptide, and a fourth polypeptide, wherein: the first polypeptide is a first light chain comprising (i) a domain selected from the group consisting of first pseudo-light chain variable domain and a first light chain variable domain, and (ii) a light chain constant domain; the second polypeptide is a second light chain comprising (i) a domain selected from the group consisting of a second pseudo-light chain variable domain and a second light chain variable domain, and (ii) a light chain constant domain; the third polypeptide is a heavy chain comprising a first bipartite polypeptide, wherein the component A comprises (i) a domain selected from the group consisting of a first pseudo-heavy chain variable domain and a first heavy chain variable domain and (ii) a CH1, a CH2, a CH3, a hinge region, and an Fc domain, and the component B comprises a first parental masking moiety; and the fourth polypeptide is a heavy chain comprising a bipartite polypeptide, wherein the component A comprises (i) a domain selected from the group consisting of a second pseudo-heavy chain domain and a second heavy chain variable domain, and (ii) a CH1, a CH2, a CH3, a hinge region, and an Fc domain, and the component B comprises a second parental masking moiety, with the proviso that at least one of the first domain, the second domain, the third domain, and the fourth domain is a pseudo-antibody variable domain.

In some embodiments, the method further comprising performing a plurality of cycles of steps (a)-(c).

In some embodiments, the CL in each of the plurality of cycles or subset thereof, is different. In some embodiments, the component A in each of the plurality of cycles or subset thereof, is the same. In some embodiments, the component B in each of the plurality of cycles or subset thereof, is the same. In some embodiments, the component A in each of the plurality of cycles or subset thereof, is the same, and the component B in each of the plurality of cycles or subset thereof, is the same.

In some embodiments, the method further comprises performing a plurality of cycles of steps (a)-(c), wherein in each cycle, the biological sample is incubated with one or more protease inhibitors or a combination of two or more protease inhibitors prior to step (a) and/or during step (a). In some embodiments, the protease inhibitor in each cycle of the plurality of cycles is different. In some embodiments, the QZ probe in each cycle of the plurality of cycles is the same. In some embodiments, the plurality of cycles is performed in parallel. In some embodiments, the plurality of cycles is performed in a multi-well plate. In some embodiments, the plurality of cycles is performed in series.

In some embodiments, the QZ probe comprises a plurality of distinct species of QZ probes, and wherein the biological sample is contacted with the plurality of distinct QZ probes. In some embodiments, each distinct species of QZ probe in the plurality or subset thereof comprises a CL having a different substrate. In some embodiments, measuring the quantity of one or more analytes in a sample of the incubated liquid comprises measuring a distinct signal associated with each distinct species of QZ probe in the plurality.

In some embodiments, each CL comprises a substrate for a protease selected from the group consisting of a disintegrin and metalloprotease (ADAM), a disintegrin and metalloproteinase with thrombospondin motifs (ADAMTS), an aspartate protease, an aspartic cathepsin, a caspase, a cysteine cathepsin, a cysteine proteinase, a KLK, a metalloproteinase, a matrix metalloproteinase (MMP), a serine protease, a coagulation factor protease, and a Type II Transmembrane Serine Protease (TTSP). In some embodiments, each CL comprises a substrate for at least one protease selected from the group consisting of ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathepsin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP1, MMP2, MMP3, MMP7, MMP8, MMP9, MMP10, MMP11, MMP12, MMP13, MMP14, MMPP15, MMPP16, MMP17, MMP19, MMP20, MMP23, MMP24, MMP26, MMP27, activated protein C, Cathepsin A, Cathepsin G, Chymase, FVIIa, FIXa, FXa, FXI, FXIIa, elastase, granzyme B, Guanidinobenzoatase, HtrA1, proteinase 3, neutrophil elastase, NSP4, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matriptase, TMPRSS2, TMPRSS3, TMPRSS4, TMPRSS5, TMPRSS6, TMPRSS7, TMPRSS8, TMPRSS9, TMPRSS10, and TMPRSS11. In some embodiments, each CL comprises a substrate for a serine protease. In some embodiments, each CL comprises a substrate for a matrix metalloproteinase (MMP). In some embodiments, each CL comprises a substrate for an aspartate protease, cysteine protease, or threonine protease.

In some embodiments, each CL comprises a substrate having an amino acid sequence selected from the group consisting of SEQ ID NOs:17-84.

In some embodiments, each QZ probe further comprises a detectable label. In some embodiments, the method further comprises measuring the quantity of analyte comprises using a secondary reagent that binds to at least one analyte, wherein the secondary reagent is attached to a detectable label. In some embodiments, the quantities of one or more analytes is/are determined by subjecting the sample(s) of incubated liquid to capillary electrophoresis. In some embodiments, the capillary electrophoresis is reducing capillary electrophoresis. In some embodiments, the quantities of one or more analyte is/are determined by subjecting the sample(s) of incubated liquid to a capillary electrophoresis immunoassay.

In some embodiments, the one or more measured analytes comprise an uncleaved intact bipartite polypeptide. In some embodiments, the one or more measured analytes comprise one or more species of cleaved polypeptide comprising component A or portion thereof and the uncleaved bipartite polypeptide. In some embodiments, the one or more measured analytes comprise one or more species of the cleaved polypeptide comprising component B or portion thereof and the uncleaved bipartite polypeptide. In some embodiments, the one or more measured analytes comprise one or more species of the cleaved polypeptide comprising component A or portion thereof, one or more species of the cleaved polypeptide comprising component B or portion thereof, and the uncleaved bipartite polypeptide.

In another aspect, the present disclosure provides a method of identifying a patient suitable for a treatment with a protease-activatable therapeutic molecule, the method comprising: determining the level of protease activity in a biological sample from a patient according to the method herein, wherein the protease-activatable therapeutic molecule is activated by a target protease, and wherein, if the biological sample is determined to have target-protease activity, then the patient is identified as being suitable for treatment with a protease-activatable therapeutic molecule.

In another aspect, the present disclosure provides a method of treating a patient having a disease or disorder with a protease-activatable therapeutic molecule that is activated by a target protease, the method comprising: administering to a patient having a disease or disorder a therapeutically effective amount of a protease-activatable therapeutic molecule, wherein the patient has been identified as suitable for treatment with the protease-activatable therapeutic molecule in accordance with the method herein.

In some embodiments, the patient has a disorder or a disease selected from the group consisting of a cardiovascular disease, a neoplastic disease, a neurodegenerative disease, an inflammatory disease, a skin disease, an infectious disease, a bacterial infection, a viral infection, an autoimmune disease, a metabolic disease, a hematologic disease, and a cancer. In some embodiments, the QZ probe comprises an activatable cytokine, wherein component A of the bipartite polypeptide comprises a cytokine and component B comprises a masking moiety.

In some aspects of the disclosure, samples of incubated liquid are subjected to a method selected from the group consisting of HPLC, mass spectrometry (MS), liquid chromatography (LC), MS-LC, SDS-PAGE (e.g., reducing SDS-PAGE), capillary electrophoresis (e.g., reducing SDS-capillary electrophoresis), size exclusion chromatography, and a capillary electrophoresis-based immunoassay (CEI).

In specific aspects of the disclosure, the sample(s) of incubated liquid is/are subjected to reducing SDS-capillary electrophoresis. In other aspects, the sample(s) of incubated liquid is/are subjected to CEI.

### BRIEF DESCRIPTION OF THE DRAWINGS

FIG. 1A depicts a schematic structure of an illustrative QZ probe comprising a bipartite polypeptide, A-CL-B. Components A and B are linked by a cleavable linker (CL) that is susceptible to cleavage by a protease.

FIG. 1B depicts a schematic structure of an exemplary QZ probe comprising an activatable pseudo-antibody having two antibody light chains, and two heavy chains comprising mutations in the variable region of a parental antibody. The mutations knock out the antigen-binding activity of the otherwise functional parental antibody. Each of the two light chains of the activatable pseudo-antibody corresponds to the bipartite polypeptide, B-CL-A, wherein the bipartite polypeptide/light chain comprises component A linked by a cleavable linker (CL) to component B that is a masking moiety.

FIG. 2 is a schematic of an illustrative way of applying the QZ assay to a tissue sample, in which: (1) a tissue section is laid on a slide; (2) a solution of a QZ probe is applied to the tissue and incubated for a suitable time; (3) the incubated liquid is collected; and (4) the components in the incubated liquid are separated by capillary electrophoresis. The resulting electropherogram depicts a peak at around 34 kDa, which corresponds to intact QZ probe (i.e., intact bipartite polypeptide), and a peak at around 30 kDa which corresponds to a cleaved QZ probe. The relative peak areas or heights provide an indication of the level of protease activity in the tissue sample.

FIG. 3A depicts superimposed capillary electropherograms of QZ probe, C225-Sub1, incubated with human membrane type serine protease 1 (MT-SP1) (solid line) and without MT-SP1 (a control, dashed line). The light chains (LC) of C225-Sub1 are bipartite polypeptides each having a component A that comprises a light chain variable domain and a light chain constant domain, and a component B that is a parental masking moiety (in the structure of, from N-terminus to C-terminus, B-CL-A). Each heavy chain (HC) of C225-Sub comprises a heavy chain variable domain, a CH1, a CH2, a CH3, a hinge region and an Fc domain. The control trace (dashed line) corresponds to the C225-Sub1 QZ probe without MT-SP1 incubation, indicating an intact LC peak of around 34 kDa and an intact heavy chain peak of around 61 kDa. The trace corresponding to the QZ probe incubated with MT-SP1 (solid line) shows the resulting cleavage product of the C225-Sub1 QZ probe. The peak at around 28 kDa corresponds to a cleaved LC peak. The peak at around 34 kDa corresponds to an intact LC peak that is substantially diminished relative to the control peak of around 34 kDa. The peak at around 61 kDa corresponds to an intact heavy chain peak (HC) of the same size as the control peak of around 61 kDa. A magnified window of the light chain section is displayed on the bottom.

FIG. 3B depicts superimposed capillary electropherograms of the C225-Sub1 QZ probe incubated with human matrix metalloproteinase-2 (MMP-2) (solid line) with a cleaved LC peak (around 28 kDa), an intact LC peak (around 34 kDa), and an intact heavy chain peak (around 61 kDa) and C225-Sub1 QZ probe incubated without MMP-2 (a control, dashed line) with an intact LC peak (around 34 kDa) and an intact heavy chain peak (HC, around 61 kDa) is depicted. A magnified window of the light chain section is displayed on the bottom.

FIG. 4 depicts the enzyme-linked immunosorbent assay (ELISA) results of C225 (an EGFR antibody), MC225 (a non-binding mutant of C225), and a non-EGFR antibody binding to immobilized EGFR. The lines for MC225 and the non-EGFR antibody fully overlap.

FIG. 5A is a diagram showing tissue partitioning into halves and quarters. Three human tumor serial sections were analyzed by leaving one whole (S1-4) and dividing the other two into halves (S1-2 and S3-4) or quarters (S1, S2, S3, and S4). The tissue areas of each section are indicated in mm².

FIG. 5B is a graph depicting protease activity of a tissue sample assessed in a constant assay volume but at different section sizes as indicated in FIG. 5A as whole (S1-4), halves (S1-2 and S3-4) or quarters (S1, S2, S3, and S4).

FIG. 5C is a graph depicting protease activity of tissue samples of the same section size assessed in different assay volumes (100 μL and 300 μL).

FIG. 5D is a graph depicting protease activity of tissue samples assessed at different section thicknesses (25 μM, 12 μM, and 4 μM) in a constant assay volume (100 μL).

FIG. 5E is a graph depicting protease activity of tissue samples assessed before and after tissue storage at −80° C. for 4 months.

FIG. 5F is a graph depicting protease activity of H292-derived xenograft tissue samples, H292 #1 and H292 #2, subjected to different freezing techniques and storage temperatures. Samples frozen using dry ice (CO₂), liquid nitrogen (LN), or −80° C. temperature were stored at −80° C. Samples frozen using −20° C. temperature were stored at −20° C.

FIG. 6 depicts superimposed capillary electropherograms of MC225-Sub1 QZ probe after 24-hour incubation on tissue samples, with QZ probe concentrations of 5 μg/mL, 10 μg/mL, 20 μg/mL or 40 μg/mL in the mixture. The lower molecular weight peaks correspond to the cleaved LC of the MC225-Sub1 QZ probe, and the higher molecular weight peaks correspond to the intact LC of the MC225-Sub1 QZ probe as indicated.

FIG. 7 depicts protease activity of H292-derived xenograft tissue samples incubated with different QZ probes. Probes A, B and C differ from each other in that each comprise a different substrate to target different proteases. H292 tumor samples from five mice were assessed with each probe.

FIG. 8A depicts protease activity of H292-derived xenograft tissue sample 5.1 incubated with different QZ probes (each having a light chain bipartite polypeptide) and different protease inhibitors. Probes A, B and C which target different proteases were used in the assessment. All the tissue samples were serially sectioned from the same H292-derived xenograft tissue sample 5.1, and incubated with QZ probe A, B or C individually. Protease activity was characterized in the absence of a protease inhibitor (Neg), or in the presence of a broad-spectrum protease inhibitor (EDTA), an MMP protease inhibitor (Galardin), or a serine protease inhibitor (aprotinin), respectively.

FIG. 8B depicts protease activity of H292-derived xenograft tissue sample 5.2 incubated with different QZ probes and different protease inhibitors. Probes A, B and C which target different proteases were used in the assessment. All the tissue samples were serially sectioned from the same H292-derived xenograft tissue sample 5.2, and incubated with QZ probe A, B or C individually. Protease activity was characterized in the absence of a protease inhibitor (Neg), or in the presence of a broad-spectrum protease inhibitor (EDTA), an MMP protease inhibitor (Galardin), or a serine protease inhibitor (aprotinin), respectively.

FIG. 9A depicts protease activity of H292-derived xenograft tissue sample 21.2 incubated with different QZ probes (each having light chain bipartite polypeptides) and different protease inhibitors. Probes D, E and F which target different proteases were used in the assessment. All tissue samples were serially sectioned from H292-derived xenograft tissue sample 21.2, and incubated with QZ probe D, E or F individually. Protease activity was characterized in the absence of a protease inhibitor (Neg), or in the presence of a broad-spectrum protease inhibitor cocktail (HALT+EDTA), a serine protease inhibitor (aprotinin), an MMP protease inhibitor (Galardin), or a cysteine protease inhibitor (E64), respectively.

FIG. 9B depicts protease activity of H292-derived xenograft tissue sample 21.3 incubated with different QZ probes and different protease inhibitors. Probes D, E and F which target different proteases were used in the assessment. All the tissue samples were serially sectioned from the same H292-derived xenograft tissue sample 21.3, and incubated with QZ probe D, E or F individually. Protease activity was characterized in the absence of a protease inhibitor (Neg), or in the presence of a broad-spectrum protease inhibitor cocktail (HALT+EDTA), a serine protease inhibitor (aprotinin), an MMP protease inhibitor (Galardin), or a cysteine protease inhibitor (E64), respectively.

FIG. 9C depicts protease activity of H292-derived xenograft tissue sample 21.5 incubated with different QZ probes and different protease inhibitors. Probes D, E and F which target different proteases were used in the assessment. All the tissue samples were serially sectioned from the same H292-derived xenograft tissue sample 21.5, and incubated with QZ probe D, E or F individually. Protease activity was characterized in the absence of a protease inhibitor (Neg), or in the presence of a broad-spectrum protease inhibitor cocktail (HALT+EDTA), a serine protease inhibitor (aprotinin), a MMP protease inhibitor (Galardin), or a cysteine protease inhibitor (E64), respectively.

FIG. 10A depicts protease activity of human head and neck squamous cell carcinoma (HNSCC) tissue samples incubated with different QZ probes and different protease inhibitors. QZ probes C225-S01 and C225-M01 (each of which having light chain bipartite polypeptides), which target serine protease and matrix metalloproteinases (MMPs) respectively, were incubated with HNSCC tissue sections. Protease activity was characterized in the absence of a protease inhibitor (no inhibitor), or in the presence of a serine protease inhibitor (aprotinin), a MMP protease inhibitor (Galardin), or a broad-spectrum protease inhibitor cocktail (HALT/EDTA), respectively.

FIG. 10B depicts protease activity of human pancreatic cancer tissue samples incubated with different QZ probes and different protease inhibitors. QZ probes C225-S01 and C225-M01, which target serine protease and matrix metalloproteinases (MMPs) activities respectively, were incubated with tissue sections of pancreatic cancer. Protease activity was characterized in the absence of a protease inhibitor (no inhibitor), or in the presence of a serine protease inhibitor (aprotinin), a MMP protease inhibitor (Galardin), or a broad-spectrum protease inhibitor cocktail (HALT/EDTA), respectively.

FIG. 10C depicts protease activity of human prostate cancer tissue samples incubated with different QZ probes and different protease inhibitors. QZ probes C225-S01 and C225-M01, which target serine protease and matrix metalloproteinases (MMPs) activities respectively, were incubated with tissue sections of prostate cancer. Protease activity was characterized in the absence of a protease inhibitor (no inhibitor), or in the presence of a serine protease inhibitor (aprotinin), a MMP protease inhibitor (Galardin), or a broad-spectrum protease inhibitor cocktail (HALT/EDTA), respectively.

FIG. 11 depicts protease activity of recombinant proteases MMP-2 and MT-SP1 cross-tested by QZ probes C225-M01 and C225-S01. High MMP-2 protease activity was detected by C225-M01 but not by C225-S01. High MT-SP1 protease activity was detected by C225-S01 but not by C225-M01.

FIG. 12 depicts k_{cat}/K_{M} of the S01 substrate in an internally quenched (IQ) probe format verse a QZ probe format C225-S01. The S01 substrate in the context of an IQ probe, a small probe, indicates a higher k_{cat}/K_{M} to both uPA and MT-SP1 proteases, than in the context of a QZ probe, a large probe.

FIG. 13A depicts in situ protease activity of H292 derived xenograft tumor tissues (n=5 mice) incubated with QZ probes MC225-Sub1 and MC225-Sub2 (each having light chain bipartite polypeptides). MC225-Sub2 detects a significantly higher in situ protease activity than MC225-Sub1 (P<0.001) in the H292 derived xenograft tumor tissues.

FIG. 13B depicts in vivo efficacy of activatable antibodies, QZ probes C225-Sub1 and C225-Sub2 (each having light chain bipartite polypeptide), compared to cetuximab (C225) and intravenous immunoglobulin therapy (IVIG) dosed at 10 mpk in the H292 xenograft model (n=8 mice per group). C225-Sub2 exhibits significantly higher in vivo efficacy than C225_Sub1 (P<0.001).

FIG. 14A depicts protease activity of patient tumor samples incubated with QZ probe MC225-Sub2. Tumor tissue samples and adjacent normal colon tissue samples were from four colorectal cancer (CRC) patients. A higher protease activity was observed in tumor sections compared to normal adjacent sections in all four patients.

FIG. 14B depicts protease activity of cholangiocarcinoma patient tissues (n=4) incubated with QZ probe MC225-Sub1 or MC225-Sub2. A higher protease activity rate was observed with MC225-Sub2 compared with MC255-Sub1 in the tissue samples from all four patients.

FIG. 15A depicts protease activity of two tissue sections each from two H292 derived xenograft tumor samples incubated with MC225-Sub1. There is no significant difference in protease activity from two tumors of the same mouse xenograft model.

FIG. 15B depicts protease activity of four tissue sections each from two patient bladder cancer tumor samples, incubated with MC225-Sub1. The tumor samples from patient #1 indicates a significantly higher protease activity than the tumor samples from patient #2.

FIGS. 16A-16D depict a series of capillary electropherograms of a QZ probe comprising an activatable anti-PD1 antibody incubated in a plasma sample. This QZ probe has two light chain bipartite polypeptides (LC) each with the same component A comprising a light chain variable domain and light chain constant domain and the same component B comprising a masking moiety in the structure of, from N-terminus to C-terminus, B-CL-A. FIG. 16A is a superimposed capillary electropherogram indicating a mix of cleaved LC and intact LC at a 2:8 ratio verses an intact LC only. FIG. 16B is a capillary electropherogram of the same QZ probe incubated in normal plasma, displaying a single intact LC peak. FIG. 16C is a capillary electropherogram of the same QZ probe incubated in plasma from a lung cancer patient, displaying a single intact LC peak. FIG. 16D is a capillary electropherogram of the QZ probe incubated in plasma from a gastric cancer patient, displaying a single intact LC peak.

FIG. 17A depicts a schematic structure of an illustrative IFN-α2b QZ probe. The IFN-α2b QZ probe has a peptide masking moiety (Affinity Mask) fused to the N-terminus of human IFN-α2b via a protease-cleavable linker and a constant fragment (Fc) masking moiety (Steric Mask) fused to the C-terminus of human IFN-α2b through a second protease-cleavable linker.

FIG. 17B is a schematic of an illustrative QZ assay using a fluorescently labeled IFN-α2b QZ probe incubated with a tissue sample.

FIG. 18A depicts capillary electropherograms of the IFN-α2b QZ probe incubated with TNBC tumor tissue sections for different time points. The light grey line corresponds to a control, the IFN-α2b QZ probe without tumor tissue incubation, indicating the intact peak of the probe. The dark grey line corresponds to the resulting cleavage product of the IFN-α2b QZ probe after a 2-hour incubation with the tumor tissue section, indicating that the probe has its peptide mask cleaved and a small amount of the probe has its Fc mask cleaved. The black line corresponds to the resulting cleavage product of the IFN-α2b QZ probe after a 16-hour incubation with the tumor tissue sections, indicating an increase in Fc mask removal.

FIG. 18B depicts interferon functional activities of the cleavage product of the IFN-α2b QZ probe incubated with TNBC tumor tissue sections for different time points. The IFN functional assay was performed with a HEK-Blue™ reporter cell line. The diamond grey line corresponds to the IFN-α2b QZ probe without TNBC tumor tissue incubation, indicating low interferon reporter activity. The triangle grey line corresponds to the resulting cleavage product of the IFN-α2b QZ probe after a 2-hour incubation with the tumor tissue section, indicating an increasing interferon reporter activity. The circle black line corresponds to the resulting cleavage product of the IFN-α2b QZ probe after a 16-hour incubation with the tumor tissue section, indicating the highest interferon reporter activity.

The figures herein are for illustrative purposes only and are not necessarily drawn to scale.

### DETAILED DESCRIPTION

The present disclosure provides a novel assay (referred to herein as the “QZ assay”) for identifying and assessing protease activity (e.g., endogenous protease activity) in a sample (e.g., a biological sample). Knowing which proteases are active in the localized area of interest could help advance the design of various protease-activatable therapeutics. Protease-activatable therapeutics are designed to activate under very specific physiological and pathological conditions in which protease dysregulation may occur. The present disclosure also provides methods for identifying whether a patient afflicted with a disease or disorder is likely to benefit from a particular protease-activatable therapeutic. Each of the methods described herein employs a specific type of probe (“QZ probe”) to ascertain the type and level of protease activity in a biological sample. In one aspect, the present disclosure provides a method of determining the level of protease activity in a biological sample, the method comprising:
(a) contacting a biological sample with a solution comprising a QZ probe to form a mixture, wherein the QZ probe comprises at least one bipartite polypeptide having a component A, a CL, and a component B in a structural arrangement of, from N-terminus to C-terminus, A-CL-B or B-CL-A,
wherein component A and component B are each independently a polypeptide,
wherein CL is a cleavable linker comprising a substrate for a protease,
wherein cleavage of the CL generates a cleavage product comprising cleaved polypeptide comprising component A or a portion thereof and cleaved polypeptide comprising component B or a portion thereof (the contacting step);
(b) incubating the mixture, thereby forming an incubated mixture comprising an incubated liquid (the incubating step); and
(c) measuring the quantity of one or more analyte in a sample of the incubated liquid to determine the level of protease activity in the biological sample, wherein the analyte is selected from the group consisting of cleaved polypeptide comprising component A or portion thereof, cleaved polypeptide comprising component B or portion thereof, intactuncleaved bipartite polypeptide, and any combination of two or more thereof. The terms “polypeptide” and “peptide” are used interchangeably herein to refer to molecule or substituent having two or more amino acids linked together via peptide bonds.

As used herein, a “biological sample” refers to a sample containing a tissue, organ, or cell (including whole cells and/or live cells and/or cell debris). The biological sample may contain (or be derived from) a “bodily fluid”. The present invention encompasses embodiments wherein the bodily fluid is selected from amniotic fluid, aqueous humour, vitreous humour, bile, blood, blood plasma, blood serum, breast milk, cerebrospinal fluid, cerumen (earwax), chyle, chyme, endolymph, perilymph, exudates, feces, ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

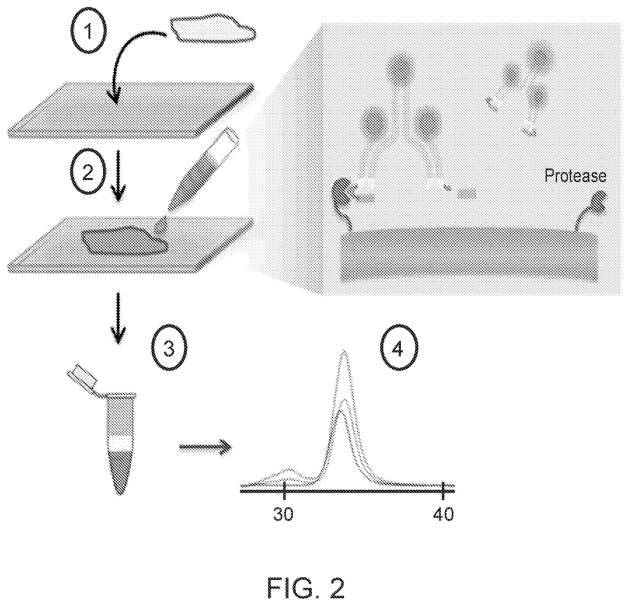

The methods described herein may be used to determine protease activity (e.g., endogenous protease activity) in any of a number of different types of biological samples, such as, for example, a tissue sample, a sample of cells, an organoid, a cell culture supernatant, a cell lysate supernatant, an organoid culture supernatant, blood, bile, bone marrow aspirate, breast milk, cerebrospinal fluid, plasma, saliva, serum, sputum, synovial fluid, urine, and the like. In a specific aspect, the present disclosure provides a method of determining the level of protease activity in a tissue sample, the method comprising:
(a) contacting a biological sample with a solution comprising a QZ probe to form a mixture, wherein the biological sample is a tissue sample, wherein the QZ probe comprises at least one bipartite polypeptide having a component A, a CL, and a component B in a structural arrangement of, from N-terminus to C-terminus, A-CL-B or B-CL-A,
wherein component A and component B are each independently a polypeptide,
wherein CL is a cleavable linker comprising a substrate for a protease,
wherein cleavage of the CL generates a cleavage product comprising cleaved polypeptide that comprises component A or a portion thereof and cleaved polypeptide comprising component B or a portion thereof (the contacting step);
(b) incubating the mixture, thereby forming an incubated mixture comprising the tissue sample and an incubated liquid (the incubating step); and
(c) measuring the quantity of one or more analyte in a sample of the incubated liquid to determine the level of protease activity in the tissue sample, wherein the analyte is selected from the group consisting of cleaved polypeptide comprising component A or portion thereof, cleaved polypeptide comprising component B or portion thereof, intactuncleaved bipartite polypeptide, and any combination of two or more thereof. A schematic of an illustrative embodiment of this method is depicted in FIG. 2.

In a further specific aspect, the present disclosure provides a method of determining the level of protease activity in a biological sample comprising a liquid, the method comprising:
(a) contacting a biological sample with a QZ probe to form a mixture, wherein the biological sample comprises a liquid, and wherein the QZ probe comprises at least one bipartite polypeptide having a component A, a CL, and a component B in a structural arrangement of, from N-terminus to C-terminus, A-CL-B or B-CL-A,
wherein component A and the component B are each independently a polypeptide,
wherein CL is a cleavable linker comprising a substrate for a protease,
wherein cleavage of the CL generates a cleavage product comprising a cleaved polypeptide comprising one or more cleaved polypeptide species comprising component A or a portion thereof and a cleaved polypeptide comprising component B or a portion thereof (the contacting step);
(b) incubating the mixture (the incubating step); and
(c) measuring the quantity of one or more analyte in a sample of liquid from the incubated mixture to determine the level of protease activity in the biological sample, wherein the analyte is selected from the group consisting of cleaved polypeptide comprising component A or portion thereof, cleaved polypeptide comprising component B or portion thereof, intact (i.e., uncleaved) bipartite polypeptide, and any combination of two or more thereof. Exemplary biological samples that comprise a liquid that may be employed herein, include, for example, a cell culture supernatant, a cell lysate supernatant, an organoid culture supernatant, blood, bile, bone marrow aspirate, breast milk, cerebrospinal fluid, plasma, saliva, sputum, synovial fluid, urine, and the like. In certain embodiments, the biological sample is a liquid obtained via a solid-liquid separation process, such as, for example, centrifugation, filtration, and the like. Often, the biological sample is plasma. In some examples, the biological sample is a sample of cells, an organoid, or a tissue sample.

When the bipartite polypeptide (and hence, the QZ probe) comprises a CL having a substrate for a protease present in the biological sample, contact and subsequent incubation of the biological sample with the QZ probe leads to cleavage (i.e., protease-mediated hydrolysis) of the CL. Cleavage of the bipartite polypeptide results in the generation of a cleavage product comprising fragments of the bipartite polypeptide. The fragments (collectively, “cleaved polypeptides”) include cleaved polypeptide species that comprise component A or portion thereof and cleaved polypeptide species comprising component B or portion thereof. Heterogeneous populations of species may result, for example, in circumstances where the CL comprises more than one protease substrate. As used herein, the terms “bipartite polypeptide”, “intact bipartite polypeptide”, “uncleaved bipartite polypeptide” and “intact uncleaved bipartite polypeptide” are used interchangeably to refer to a fusion polypeptide of two polypeptides, component A and component, B linked together via a cleavable linker.

In some embodiments, the detection of no or substantially low quantities of cleavage product and/or a relatively much larger quantity of intact/uncleaved bipartite polypeptide in the sample of incubated liquid may be an indication of either no or relatively low quantities of the protease(s) specific for the CL substrate employed in the bipartite polypeptide of the QZ probe. In some embodiments, the detection of no or substantially low quantities of cleavage product and/or a relatively much larger quantity of intact/uncleaved bipartite polypeptide in the sample of incubated liquid may be an indication of high protease inhibitor (e.g., when the assay measures activity). The method can be repeated using one or a panel of a plurality of additional QZ probes in which within each QZ probe, component A and component B of the bipartite polypeptide in each of the probes in the panel are identical with the exception that each CL comprises a substrate that differs from the substrates in the other QZ probes in the panel. Thus, each QZ probe in the panel targets a different protease. This methodology is useful for determining the identity of protease(s) present in the biological sample. When the sample of incubated liquid comprises one or more species of cleaved polypeptide, the identity of the endogenous protease present in the biological sample can be identified by knowing which substrate was employed in the bipartite probe.

The methods of the present invention are thus very useful as an indirect way for assaying for the presence of any one of a multitude of different protease activities that may be present in the biological sample of interest. In an illustrative embodiment, the method further comprises repeating an initial cycle of the steps (a)-(c) above, wherein the QZ probe in each cycle of a plurality of cycles comprises a bipartite polypeptide having a structural arrangement of, from N-terminus to C-terminus, A-CL-B or B-CL-A, as defined above. In some embodiments, the CL in each of the plurality of cycles or subset thereof, is different. In some embodiments, component A in each of the plurality of cycles or subset thereof, is the same and/or component B in each of the plurality of cycles or subset thereof, is the same. In certain embodiments, component A in each of the plurality of cycles or subset thereof, is the same, and component B in each of the plurality of cycles or subset thereof, is the same.

In some instances where there is a possibility of more than one protease species contributing to cleavage (due to, for example, the CL comprising two or more substrates for two or more protease species), the method further comprises pre-incubating the biological sample with one or more protease inhibitors prior to the contacting step. Therefore, in certain embodiments, the method comprises repeating an initial cycle of steps (a)-(c), wherein the QZ probe in each cycle is the same, but wherein the method further comprises pre-incubating the biological sample with one or more protease inhibitors prior to the contacting step in at least one cycle. In some embodiments, in at least a subset of the plurality of cycles, each cycle comprises pre-incubating the biological sample with one or more protease inhibitors prior to the contacting step, wherein a different protease inhibitor is employed in each of these cycles. The identity of the relevant protease can be deduced by noting the protease inhibitor condition that results in a decrease in cleavage product or commensurately, an increase in quantity of uncleaved bipartite polypeptide in the sample of incubation liquid. In some embodiments, a combination of two or more protease inhibitors is employed.

When it is desirable to test a biological sample against a plurality of diverse QZ probes, or diverse conditions (e.g., with the addition of one or more protease inhibitors (e.g., diverse protease inhibitors)), the cycles may be performed in parallel, such as for example, in a multi-well plate. Alternatively, the plurality of cycles may be performed serially. The biological sample employed in each cycle is often derived from the same biological specimen.

In a further aspect, a diverse library (i.e., a plurality) of QZ probes can be applied to a single biological sample, e.g., in step (a) the QZ probe may comprise a plurality of distinct species of QZ probes, in which the (a single) biological sample is contacted with the plurality of distinct QZ probes. In some embodiments, the QZ probe comprises an antibody or a pseudo-antibody. In certain of these embodiments, each distinct species of QZ probe comprises a distinct antibody or pseudo-antibody. In certain embodiments, each distinct species of QZ probe in the plurality or subset thereof comprises a CL having a different substrate. Measuring the quantity of one of more analytes associated with each species of QZ probe can be accomplished by measuring a unique signal associated with each distinct species of QZ probe. In some of these embodiments, the analytes can be measured in a number of different ways, such as, for example, by using anti-idiotype antibodies each specific for a distinct QZ probe, by differentially labeling the QZ probes with specific labels or dyes followed by detection, and the like. Usually the QZ probes comprise CL substituents comprising different substrates. These formats are particularly useful for carrying out relatively rapid identification of and quantitation of levels endogenous protease activity in a particular biological sample/specimen.

The level of protease activity in the biological sample can be represented by any of a variety of different metrics, e.g., the quantity(ies) (or peak area(s) or peak height(s)) corresponding to any one or more of cleaved polypeptide comprising component A or portion thereof, or the quantity (or peak area or peak height) corresponding to cleaved polypeptide comprising component B or portion thereof, or the quantity (or peak area or peak height) corresponding to (intact) bipartite polypeptide in the sample of incubated liquid; or the percentage of bipartite polypeptide in the QZ reagent converted to cleaved polypeptide comprising component A or portion thereof, and/or cleaved polypeptide comprising component B or portion thereof; or the ratio of the quantity(ies) (or peak area(s) or peak height(s)) of cleaved polypeptide comprising component A or portion thereof and/or cleaved polypeptide comprising component B or portion thereof, to the quantity (or peak area or peak height) of (intact) bipartite polypeptide in the sample of incubated liquid; or the ratio or percentage of the quantity(ies) (or peak area(s) or peak height(s)) of any one or more of the cleavage polypeptide species to (or on the basis of) the total quantity (or total peak area or total peak height) of the cleaved polypeptide species and (intact) bipartite polypeptide in the sample of incubated liquid; or the ratio of the absolute difference in the quantity (or peak area or peak height) of intact bipartite polypeptide in the reagent of step (a) and the quantity (or peak area or peak height) of intact bipartite polypeptide in the sample of incubated liquid, to the quantity (or peak area or peak height) of intact bipartite polypeptide in the sample of incubated liquid; or any other similar metric computed from the quantities of any one or more of the quantity of cleaved polypeptide comprising component A or portion thereof, the quantity of cleaved polypeptide comprising component B or portion thereof, and/or the quantity of intact bipartite polypeptide in the sample of incubated liquid and/or the quantity of intact bipartite polypeptide in the QZ reagent, wherein peak area is obtained from, for example, a chromatogram, an electropherogram, and the like.

Thus, in some embodiments, the measured analyte is an uncleaved bipartite polypeptide. In certain embodiments, the measured analytes are one or more species of cleaved polypeptide comprising component A or portion thereof and an uncleaved bipartite polypeptide. In other embodiments, the measured analytes is/are one or more species of cleaved polypeptide comprise component B or portion thereof and an uncleaved bipartite polypeptide. In other embodiments, the measured analytes are one or more species of cleaved polypeptide comprising component A or portion thereof, one or more species of cleaved polypeptide comprising component B or portion thereof, and an uncleaved bipartite polypeptide.

Any method for separating and detecting different polypeptide species in a mixture may be used to quantify the polypeptide components in the sample of incubated liquid and the quantity of (intact) bipartite polypeptide in the QZ probe reagent. Suitable methods include, for example, HPLC, mass spectrometry (MS), liquid chromatography (LC), MS-LC, SDS-PAGE (e.g., reducing SDS-PAGE), capillary electrophoresis (e.g., reducing SDS-capillary electrophoresis), size exclusion chromatography, a capillary electrophoresis-based immunoassay (CEI), and the like. Due to relatively smaller size of the cleavage products as compared to size of intact bipartite polypeptide, cleavage product and (intact) bipartite polypeptide can be readily distinguished from each other on the basis of molecular weight. In certain embodiments, the quantities of cleaved polypeptide comprising component A or portion thereof, cleaved polypeptide comprising component B or portion thereof, and/or uncleaved bipartite polypeptide are determined by a method selected from the group consisting of capillary electrophoresis and a capillary electrophoresis-based immunoassay.

In certain embodiments, capillary electrophoresis is employed. In some of these embodiments, the capillary electrophoresis method is a reducing capillary electrophoresis method. In certain other embodiments, a capillary electrophoresis-based immunoassay is used, as described, for example, in PCT Publication No. WO 2019/018828, which is incorporated herein by reference.

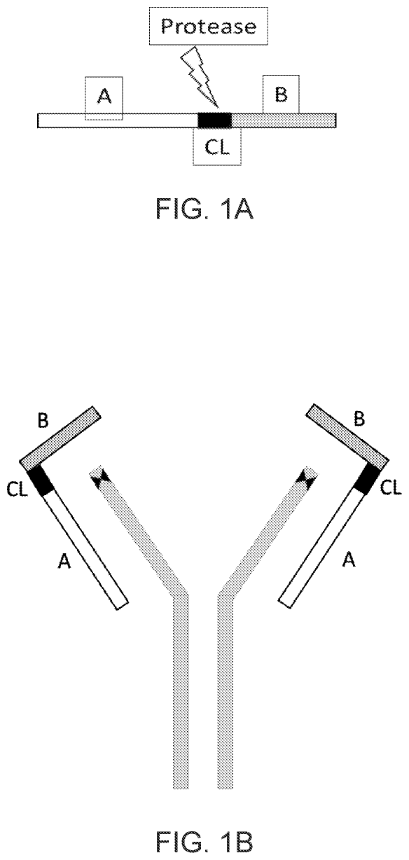

As used herein, the term “QZ probe” refers to a polypeptide or a complex of polypeptides in which at least one of the polypeptides is a bipartite polypeptide having the structure described above. In some embodiments, the QZ probe comprises a single polypeptide, i.e., the bipartite polypeptide. An illustration of a single polypeptide QZ probe is depicted in FIG. 1A. In other embodiments, the QZ probe comprises at least two polypeptides, of which at least one is a bipartite polypeptide. In certain embodiments, the QZ probe comprises three or four polypeptides, of which at least one is a bipartite polypeptide, and in some instances, at least two are bipartite polypeptides.

The QZ probe may be a binding or non-binding QZ probe as demonstrated in the Examples hereinbelow. The terms “binding” and “non-binding” refer to the capacity for the QZ probe to bind to a target in the biological sample. Whether or not a QZ probe binds can be readily determined by methods that are well known in the art, including by an ELISA assay, and the like. The QZ assay is often performed under non-binding conditions. As used herein, the term “non-binding conditions” refers to conditions that result in no or substantially reduced binding of the QZ probe to a target in the biological sample. Non-binding conditions may comprise use of a non-binding QZ probe, or employing the addition step of blocking the binding targets in the biological sample prior to contacting the biological sample with the QZ probe. Techniques for blocking biological targets include use of a blocking agent, such as, for example, an antibody that binds to the target, and the like, or by using other techniques, such as, for example using an excess of the QZ probe to compensate for loss of some of the QZ probe to binding, as well as any known technique for blocking targets in a biological sample.

The QZ probe employed herein may comprise one polypeptide, for example, a bipartite polypeptide as depicted in FIG. 1A, with a structure arrangement of A-CL-B, from N-terminus to C-terminus. Alternatively, the QZ probe may comprise a complex of two, three, four, or more polypeptides of which at least one polypeptide is a bipartite polypeptide.

The bipartite polypeptide may further comprise one or more linkers, such as a flexible linker disposed between component A and the CL, and/or the CL and component B; and in the opposite orientation, between component B and the CL, and/or the CL and component A. One or more of such linkers may be employed to provide spatial separation between two or more elements in the bipartite polypeptide.

Linkers that are suitable for use as flexible linkers in the bipartite polypeptide include any that are known in the art. Specific flexible linkers that are suitable for use in the bipartite polypeptide include any of those described in, e.g., PCT publication number WO 2021/207669, which is incorporated herein by reference. Exemplary linkers include those comprising or consisting of glycine; glycine and serine; and glycine, serine and at least one of alanine or threonine. Such linkers may be from about 1 to about 30, from 1 to about 25, etc. amino acid residues in length. In some embodiments, the linkers consists of a glycine residue. In some embodiments, the linker is a polypeptide consisting of glycine residues. In some embodiments, linker can be rich in glycine (Gly or G) residues. In some embodiments, the linker can be rich in serine (Ser or S) residues. In some embodiments, the linker can be rich in glycine and serine residues. In some embodiments, the linker has one or more glycine-serine residue pairs (GS) (e.g., 1, 2, 3, 4, 5, 6, 7, 8, 9, or 10 or more GS pairs).

Bipartite polypeptides suitable for use in the present invention may have a molecular weight of at least about 1 kDa, or at least about 5 kDa or at least about 10 kDa, or at least about 20 kDa, or at least about 30 kDa, or at least about 40 kDa, or at least about 50 kDa, or at least about 60 kDa, or at least about 70 kDa, or at least about 80 kDa, or at least about 90 kDa, or at least about 100 kDa. Suitable bipartite polypeptides include those having a molecular weight in the range of from about 1 kDa to about 500 kDa, or from about 5 kDa to about 400 kDa, or from about 10 kDa to about 200 kDa, or from about 15 kDa to about 150 kDa. All ranges referred to herein are intended to be inclusive of the endpoints that define the range.

As used in the present disclosure, the term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

In other embodiments, the bipartite polypeptide may comprise at least about 10 amino acid residues, or at least about 50 amino acid residues, or at least about 100 amino acid residues, or at least about 200 amino acid residues, or at least about 300 amino acid residues, or at least about 400 amino acid residues, or at least about 500 amino acid residues, or at least about 600 amino acid residues, or at least about 700 amino acid residues, or at least about 800 amino acid residues, or at least about 900 amino acid residues, or at least about 1000 amino acid residues. In certain embodiments, the bipartite polypeptide comprises from about 10 to about 5000 amino acid residues, or from about 50 to about 4000 amino acid residues, or from about 100 to about 2000 amino acid residues, or from about 150 to about 1500 amino acid residues.

Components A and B deployed within the bipartite polypeptide may have the same amino acid sequence or may have different amino acid sequences (e.g., different with respect to sequence composition and/or sequence length). Often, component A and component B have different amino acid sequences and different sequence lengths. Correspondingly, components A and B may have the same or different molecular weight. In certain embodiments, components A and B have the same molecular weight. In other embodiments, components A and B have different molecular weights. Often, components A and B have at least about 5% difference, or at least about 10% difference, or at least 15% difference, or at least about 20% difference, or at least 25% difference, in molecular weight.

The molecular weight of each cleavage polypeptide will vary and depend on a variety of factors, including, for example, position of the substrate(s) within the bipartite polypeptide, type of substrate(s) employed, location of the cleavage site(s) within the substrate(s), molecular weight of components A and B, molecular weight of the (intact) bipartite polypeptide, and the like. Since the QZ probe can be designed with specific known substrates, it is possible to have some a priori knowledge of approximate molecular weights of the cleaved polypeptides. In some embodiments, at least one of the cleaved polypeptide comprising A or portion thereof and the cleaved polypeptide comprising B or portion thereof has a molecular weight of at least about 1 kDa, or at least about 5 kDa or at least about 10 kDa, or at least about 20 kDa, or at least about 30 kDa, or at least about 40 kDa, or at least about 50 kDa, or at least about 60 kDa, or at least about 70 kDa, or at least about 80 kDa, or at least about 90 kDa, or at least about 100 kDa. In other embodiments, at least one of the cleaved polypeptide comprising component A or a portion thereof and the cleaved polypeptide comprising component B or a portion thereof has a molecular weight in the range of from about 1 kDa to about 500 kDa, or from about 5 kDa to about 400 kDa, or from about 10 kDa to about 200 kDa, or from about 15 kDa to about 150 kDa.

In certain embodiments, at least one of the cleaved polypeptide comprising component A or a portion thereof and the cleaved polypeptide comprising component B or a portion thereof comprises at least about 10 amino acid residues, or at least about 50 amino acid residues, or at least about 100 amino acid residues, or at least about 200 amino acid residues, or at least about 300 amino acid residues, or at least about 400 amino acid residues, or at least about 500 amino acid residues, or at least about 600 amino acid residues, or at least about 600 amino acid residues, or at least about 700 amino acid residues, or at least about 800 amino acid residues, or at least about 900 amino acid residues, or at least about 1000 amino acid residues. At least one of the cleaved polypeptides may have a sequence length in the range of from about 10 to about 5000 amino acid residues, or from about 50 to about 4000 amino acid residues, or from about 100 to about 2000 amino acid residues, or from about 150 to about 1500 amino acid residues.

In some embodiments, at least one of the cleaved polypeptide comprising component A or a portion thereof and the cleaved polypeptide comprising component B or a portion thereof has a molecular weight that is in the range of about 5% to about 95% of the molecular weight of the bipartite polypeptide, or in the range of about 10% to about 90% of the molecular weight of the bipartite polypeptide, or in the range of about 20% to about 80% of the molecular weight of the bipartite polypeptide, or in the range of about 30% to about 70% of the molecular weight of the bipartite polypeptide.

In some embodiments, the QZ probe comprises an antibody. As used herein, the term “antibody” refers to an immunoglobulin (Ig) molecule or an immunologically active portion of Ig molecule, i.e., a molecule that contains an antigen binding domain that specifically binds an antigen. In some embodiments, the antibody comprises two light chains and two heavy chains. As used herein, the terms “light chain” and “LC” are used interchangeably herein to refer to a polypeptide comprising either a light chain variable domain or a pseudo-light chain variable domain and a light chain constant domain. As described in more detail herein below, the LC may comprise additional elements, such as, for example, a light chain constant domain, a masking moiety, and the like. As used herein, and the terms “heavy chain” and “HC” are used interchangeably herein to refer to a polypeptide comprising a heavy chain variable domain or a pseudo-heavy chain variable domain. The HC may comprise additional elements, such as, for example, a CH1, a CH2, and a CH3 domain, a hinge region, an Fc domain, and the like. In some instances, the bipartite polypeptide is a light chain (LC) wherein one of component A and component B comprises a light chain variable domain or a pseudo-light chain variable domain and a constant domain, and the other of component A and component B comprises a masking moiety (or parental masking moiety) as described in more detail herein below. In other instances, the bipartite polypeptide is a heavy chain (HC) wherein one of component A and component B comprises a heavy chain variable domain or a pseudo-heavy chain variable domain, a CH1, a CH2, and a CH3 domain, a hinge region, and an Fc domain, and the other of component A and component B comprises a masking moiety (or a parental masking moiety).

In other embodiments, the antibody employed herein can comprise a Fab, a F(ab′)2, a monospecific Fab2, a bispecific Fab2, a trispecific Fab3, an scFv, a bispecific diabody, a trispecific triabody, an scFv-Fc, a minibody, a bispecific T cell engager (e.g., a BiTE™), a dual-affinity re-targeting antibody (DART antibody), and the like.

In some embodiments in which the QZ probe comprises an antibody, at least one of component A and B comprises one or more antibody components selected from the group consisting of a light chain variable domain and a heavy chain variable domain, and a combination thereof. In certain of these embodiments, component A comprises a light chain variable domain. In other embodiments, component A comprises a heavy chain variable domain. In further embodiments, component A comprises both a light chain variable domain and a heavy chain variable domain.

In some embodiments, the QZ probe comprises an antibody, wherein the QZ probe comprises at least a first polypeptide comprising a bipartite polypeptide and a second polypeptide. In some of these embodiments, component A in the first polypeptide comprises a light chain variable domain and the second polypeptide comprises at least one heavy chain variable domain. In certain of these embodiments, component A further comprises a light chain constant domain and the second polypeptide further comprises one or more heavy chain constant domain(s) and/or an Fc domain, wherein the first and the second polypeptides bind to each other via one or more disulfide bonds. In other embodiments, component A in the first polypeptide comprises a heavy chain variable domain, and the second polypeptide comprises a light chain variable domain. In certain of these embodiments, component A further comprises one or more heavy chain constant domain(s) and/or a Fc domain and the second polypeptide further comprises a light chain constant domain, wherein the first and the second polypeptides bind to each other via one or more disulfide bonds. In some embodiments, component B comprises a polypeptide having from about 3 to about 200 amino acid residues. In certain of these embodiments, the molecular weight of component B is less than about 50% that of component A.

In some instances, the QZ probe comprises at least three polypeptides. In other instances, the QZ probe comprises at least four polypeptides. In some of these embodiments, the QZ comprises a first polypeptide, a second polypeptide, a third polypeptide, and a fourth polypeptide, wherein: (1) the first polypeptide comprises a first bipartite polypeptide that comprises a first component A comprising a first light chain variable domain; (2) the second polypeptide comprises a second component A comprising a second light chain variable domain; (3) the third polypeptide comprises a first heavy chain variable domain; and (4) the fourth polypeptide comprises a second heavy chain variable domain. In some of the above-described embodiments, the first and second polypeptides further comprise a light chain constant domain and the third and fourth polypeptides further comprise one or more heavy chain constant domains and/or a Fc domain, wherein the first and third polypeptides, and the second and fourth polypeptides bind to each other via one or more disulfide bonds. In some embodiments, the QZ probe comprises a bipartite polypeptide that is a light chain, wherein component A comprises a light chain variable domain and a light chain constant domain, and wherein the QZ probe comprises a second polypeptide comprising a heavy chain variable domain, a CH1, CH2, and CH3 domain, a hinge region, and an Fc domain, wherein the structure of the bipartite polypeptide is, from N-terminus to C-terminus, B-CL-A.

In some embodiments, component B comprises a polypeptide having from about 3 to about 200 amino acid residues. In certain of these embodiments, the molecular weight of component B is less than about 50% that of component A.

In some embodiments, the QZ probe comprises an antibody comprising a first polypeptide, a second polypeptide, a third polypeptide, and a fourth polypeptide, wherein: (1) the first polypeptide comprises a first bipartite polypeptide that comprises a first component A comprising a first heavy chain variable domain; (2) the second polypeptide comprises a second component A comprising a second heavy chain variable domain; (3) the third polypeptide comprises a first light chain variable domain; and (4) the fourth polypeptide comprises a second light chain variable domain. In some of these embodiments, the third and fourth polypeptides further comprise a light chain constant domain and the first and second polypeptides further comprise one or more heavy chain constant domain(s) and/or a Fc domain, wherein the first and third polypeptides and the second and fourth polypeptides bind to each other via one or more disulfide bonds. In some embodiments, the first and second polypeptides each independently are bipartite polypeptides that are light chains, wherein each component A comprises a light chain variable domain and a light chain constant domain, and wherein the third and fourth polypeptides are each a heavy chain comprising a heavy chain variable domain, a CH1, CH2, and CH3 domain, a hinge region, and an Fc domain, where the structure of the bipartite polypeptide is, from N-terminus to C-terminus, B-CL-A. In some embodiments, component B comprises a polypeptide having from about 3 to about 200 amino acid residues. In certain of these embodiments, the molecular weight of component B is less than about 50% that of component A.

In another aspect, the QZ probe comprises an activatable antibody in which each component B is a masking moiety (MM). As used herein, the terms “masking moiety” and “MM,” are used interchangeably herein to refer to a peptide that, when positioned proximal to the binding domain of an antibody, interferes with the binding of the antibody to its biological target. As used herein, a masking moiety (or MM), when positioned proximal to a receptor domain of a cytokine, reduces the cytokine activity. As used herein, the term “activatable antibody” refers to a construct comprising an antibody and a masking moiety, and the term “activatable antibody” refers to a construct comprising a cytokine and a masking moiety. In certain embodiments, the QZ probe comprises an activatable antibody, wherein the bipartite polypeptide is a light chain, wherein component A comprises a light chain variable domain and a light chain constant domain and component B comprises a masking moiety. In certain embodiments, the QZ probe comprises an activatable cytokine, wherein component A of the bipartite polypeptide comprises a cytokine and component B comprises a masking moiety.

In some embodiments, the MM is a polypeptide that is capable of binding to another component of the QZ probe. In some examples, the MM may be an antibody or antibody fragment (e.g., a Fab fragment, a F(ab′)2 fragment, a scFv, a scAb, a dAb, a single domain heavy chain antibody, and a single domain light chain antibody) that binds to another component of the QZ probe such that interrupts another QZ probe component's binding to its target. In some examples, the MM may be a ligand, a receptor, a fragment thereof (e.g., an extracellular domain of a receptor) of another component of the QZ probe that binds to the QZ probe component and interrupts the QZ probe component's binding to its target. In some examples, when the QZ probe component is an antibody or antibody fragment thereof, the MM may be an anti-idiotypic antibody or fragment thereof (e.g., scFv) that binds to the idiotype of the QZ probe component. In some examples, the MM may be a cytokine or a receptor for a cytokine. In some examples, the MM may have an amino acid sequence that is at least 85% identical to a cytokine or to a receptor for a cytokine.

In some embodiments, the MM does not bind other QZ probe component, but still interferes with other QZ probe component's binding to its binding partner through non-specific interactions such as steric hindrance. For example, the MM may be positioned in the activatable molecule such that the tertiary or quaternary structure of the activatable molecule allows the MM to mask the QZ probe component through charge-based interaction, thereby holding the MM in place to interfere with binding partner access to the QZ probe component. Examples of such MMs include an albumin, e.g., human serum albumin (HSA), a fragment crystallizable (Fc) domain, an antibody constant domain (e.g., CH domains), a polymer (e.g., branched or multi-armed polyethylene glycol (PEG)), a latency associated protein (LAP), and any polypeptide or other moieties that sterically interfere the interaction between another QZ probe component and its target. In some examples, the MM may recruit a large protein binding partner that sterically interfere the interaction between another QZ probe component and its target. For example, the MM may be an antibody or a fragment thereof that binds to serum albumin.

Examples of suitable masking moieties include the full-length or a fragment or mutein of a cognate receptor of another QZ component, and antibodies and fragment thereof, e.g., a polyclonal antibody, a recombinant antibody, a human antibody, a humanized antibody a single chain variable fragment (scFv), single-domain antibody such as a heavy chain variable domain (VH), a light chain variable domain (VL), a variable domain of camelid-type nanobody (VHH), a dAb and the like. Other exemplary antigen-binding domain that bind another QZ component can also be used as an MM include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. As another example, a peptide that is modified by conjugation to a water-soluble polymer, such as PEG, can sterically inhibit or prevent binding of the cytokine to its receptor. Antibodies and antigen-binding domains that bind to, for example, a protein with a long serum half-life such as HSA, immunoglobulin or transferrin, or to a receptor that is recycled to the plasma membrane, such as FcRn or transferrin receptor, can also inhibit the cytokine, particularly when bound to their antigen.

When the QZ probe comprises an EGFR antibody, a mutant thereof, or a fragment thereof, the QZ probe may comprise a MM comprising any one of SEQ ID NOs: 86-127. When the QZ probe comprises an interferon, a mutant thereof, or a fragment thereof, the QZ probe may comprise a MM comprising any one of SEQ ID NOs: 128-162.

In certain embodiments, the QZ probe comprises an activatable antibody comprising a first polypeptide, a second polypeptide, a third polypeptide, and a fourth polypeptide as described hereinabove, wherein the first and second component B of the above-described bipartite polypeptides each independently comprise a masking moiety. In some aspects, the QZ probe comprises an activatable antibody comprising a first polypeptide, a second polypeptide, a third polypeptide, and a fourth polypeptide, wherein:
the first polypeptide is a first light chain comprising a first bipartite polypeptide, wherein component A comprises a first light chain variable domain, and (ii) a light chain constant domain, and component B comprises a first masking moiety;
the second polypeptide is a second light chain comprising a second bipartite polypeptide, wherein component A comprises a second light chain variable domain, and (ii) a light chain constant domain, and component B comprises a second masking moiety;
the third polypeptide is a first heavy chain comprising a first heavy chain variable domain, and (ii) a CH1, a CH2, a CH3, a hinge region, and an Fc domain; and
the fourth polypeptide is a second heavy chain comprising (a second heavy chain variable domain, and (ii) a CH1, a CH2, a CH3, a hinge region, and an Fc domain. In some of the above-described embodiments, the bipartite polypeptides have the structure of, from N-terminus to C-terminus, B-CL-A.

In other aspects, the QZ polypeptide comprises an activatable antibody comprising a first polypeptide, a second polypeptide, a third polypeptide, and a fourth polypeptide, wherein:
the first polypeptide is a first light chain comprising a first light chain variable domain, and a light chain constant domain;
the second polypeptide is a second light chain comprising a second light chain variable domain and a light chain constant domain;
the third polypeptide is a first heavy chain comprising a first bipartite polypeptide, wherein component A comprises a first heavy chain variable domain and a CH1, a CH2, a CH3, a hinge region, and an Fc domain, and component B comprises a first masking moiety; and
the fourth polypeptide is a second heavy chain comprising a bipartite polypeptide, wherein component A comprises a second heavy chain variable domain, and a CH1, a CH2, a CH3, a hinge region, and an Fc domain, and component B comprises a second masking moiety. In some of the above-described embodiments, the bipartite polypeptides have the structure of, from N-terminus to C-terminus, B-CL-A.

In some embodiments, the QZ probe comprises a pseudo-antibody or pseudo-light, or pseudo-heavy domain thereof. As used herein, the term “pseudo-antibody” refers to a compound that has the structure of an antibody, but which differs from the antibody in that it exhibits weak or no detectable binding to the biological sample as measured by, for example, an ELISA. A pseudo-antibody may be a mutated version (i.e., a variant) of a parental antibody in which the mutations cause the parental antibody to lose some or all of its ability to bind to its biological target.

As used herein, the term, “pseudo-light chain variable domain” refers to a light chain variable domain that has been mutated relative to the light chain variable domain of a parental antibody, where such mutation(s) impair(s) the ability of the antibody to bind to its target. In some instances, a pseudo-light chain variable domain comprises one or more mutations in the complementarity-determining regions (CDRs) of the parental antibody light chain variable domain.

The term, “pseudo-heavy chain variable domain” is used herein to refer to a heavy chain variable domain that has been mutated relative to the heavy chain variable domain of a parental antibody, such that the mutation(s) impair(s) the ability of the antibody to bind to its target. In some instances, a pseudo-heavy chain variable domain comprises one or more mutations in the complementarity-determining regions (CDRs) of the parental antibody heavy chain variable domain. A “pseudo-antibody variable domain” refers to either of both a pseudo-light chain variable domain and/or a pseudo-heavy chain variable domain.

A pseudo-antibody comprises at least one of a pseudo-light chain variable domain and a pseudo-heavy chain variable domain. In some embodiments, a pseudo-antibody comprises a pseudo-light chain variable domain and a heavy chain variable domain. In other embodiments, the pseudo-antibody comprises a light chain variable domain and a pseudo-heavy chain variable domain. In some embodiments, the pseudo-antibody comprises a pseudo-light chain variable domain and a pseudo-heavy chain variable domain.

In some embodiments, the QZ probe comprises a pseudo-antibody, wherein one of component A and component B comprises a pseudo-antibody variable domain selected from the group consisting of a pseudo-light chain variable domain and a pseudo-heavy chain variable domain, or a combination thereof. In some of these embodiments, component A comprises the pseudo-antibody variable domain.

In a specific embodiment, the QZ probe comprises a bipartite polypeptide and a second polypeptide, wherein component A comprises a pseudo-light chain variable domain, and the second polypeptide comprises a heavy chain variable domain. In other embodiments, the QZ probe comprises a bipartite polypeptide and a second polypeptide wherein component A comprises a light chain variable domain, and the second polypeptide comprising a pseudo-heavy chain variable domain.

In a specific embodiment, the QZ probe comprises a pseudo-antibody comprising the bipartite polypeptide and a second polypeptide, wherein either
(i) component A comprises a pseudo-light chain variable domain and the second polypeptide comprises a domain selected from the group consisting of a heavy chain variable domain and a pseudo-heavy chain variable domain; or
(ii) component A comprises a pseudo-heavy chain variable domain and the second polypeptide comprises a domain selected from the group consisting of a light chain variable domain and a pseudo-light chain variable domain.

In a further embodiment, the QZ probe comprises a pseudo-antibody comprising the bipartite polypeptide and a second polypeptide, wherein either
(i) component A comprises a light chain variable domain and the second polypeptide comprises a pseudo-heavy chain domain; or
(ii) component A comprises a heavy chain variable domain and the second polypeptide comprises a pseudo-light chain variable domain. In some embodiments, component B comprises a polypeptide having from about 3 to about 200 amino acid residues. In certain of these embodiments, the molecular weight of component B is less than about 50% that of component A.

The above-described QZ probes may further comprise a third polypeptide comprising one or more of a light chain variable domain, a pseudo-light chain variable domain, a heavy chain variable domain, a pseudo-heavy chain variable domain, and combinations of any two or more thereof.

In some embodiments, the QZ probe comprises a pseudo-antibody, wherein the QZ probe comprises at least four polypeptides. In some of these embodiments, the QZ probe comprises a first polypeptide, a second polypeptide, a third polypeptide, and a fourth polypeptide, wherein:
the first polypeptide comprises a first bipartite polypeptide that comprises a first component A which comprises a domain selected from the group consisting of a first light chain variable domain and a first pseudo-light chain variable domain;
the second polypeptide comprising a second component A which comprises a domain selected from the group consisting of a second light chain variable domain and a second pseudo-light chain variable domain;
the third polypeptide comprising a first pseudo-heavy chain variable domain; and
a fourth polypeptide comprising a second pseudo-heavy chain variable domain.

In other of these embodiments, the QZ probe comprises a first polypeptide, a second polypeptide, a third polypeptide, and a fourth polypeptide, wherein:
the first polypeptide comprises a first bipartite polypeptide that comprises a first component A which comprises a first pseudo-light chain variable domain;
the second polypeptide comprising a second component A which comprises a second pseudo-light chain variable domain;
the third polypeptide comprising a first heavy chain variable domain; and
the fourth polypeptide comprising a second heavy chain variable domain.

In some of the above-described embodiments, the first and second polypeptides further comprise a light chain constant domain. In some of these embodiments, the third and fourth polypeptides further comprise one or more heavy chain constant domains and/or a Fc domain. In some embodiments, the first and second polypeptides each independently comprise a component A that comprises a light chain and the third and fourth polypeptides each independently comprise a heavy chain, where the structure of the bipartite polypeptide is, from N-terminus to C-terminus, B-CL-A. In some embodiments, each component B independently comprises a polypeptide having from about 3 to about 200 amino acid residues. In certain of these embodiments, the molecular weight of component B is less than about 50% that of component A.

In some embodiments, the QZ probe comprises a pseudo-antibody having a first polypeptide, a second polypeptide, a third polypeptide, and a fourth polypeptide, wherein:
the first polypeptide comprises a first bipartite polypeptide that comprises a first component A which comprises a domain selected from the group consisting of a first heavy chain variable domain and a first pseudo-heavy chain variable domain;
the second polypeptide comprising a second component A which comprises a domain selected from the group consisting of a second heavy chain variable domain and a second pseudo-heavy chain variable domain;
the third polypeptide comprising a first pseudo-light chain variable domain; and
the fourth polypeptide comprising a second pseudo-light chain variable domain.

In other embodiments, the QZ comprises a pseudo-antibody comprising a first polypeptide, a second polypeptide, a third polypeptide, and a fourth polypeptide, wherein:
the first polypeptide comprises a first bipartite polypeptide that comprises a first component A which comprises a first pseudo-heavy chain variable domain;
the second polypeptide comprises a second component A which comprises a second pseudo-heavy chain variable domain;
the third polypeptide comprising a first light chain variable domain; and
the fourth polypeptide comprising a second light chain variable domain. In certain embodiments, the first and second polypeptide further comprise at least one heavy chain constant domain and/or an Fc domain, and the third and fourth polypeptides comprise a light chain constant domain. In some of the above-described embodiments, the first and second component A each independently comprise a heavy chain, and the third and fourth polypeptides each comprise a light chain, where the structure of the bipartite polypeptide is, from N-terminus to C-terminus, B-CL-A. In some embodiments, component B comprises a polypeptide having from about 3 to about 200 amino acid residues. In certain of these embodiments, the molecular weight of component B is less than about 50% that of component A.

In another aspect of the present disclosure, the pseudo-antibody is a variant of a parental antibody, and component B of the bipartite polypeptide is a masking moiety (MM) for the parental antibody (i.e., a “parental masking moiety”). As used herein, the term “activatable pseudo-antibody” refers to a construct comprising a pseudo-antibody and a masking moiety in which the pseudo-antibody is a variant of a parental antibody and the masking moiety is a parental masking moiety. In certain embodiments, the QZ probe comprises an activatable pseudo-antibody, wherein the antibody component of the activatable pseudo-antibody is a variant of a parental antibody, wherein the bipartite polypeptide is a light chain, wherein component A comprises a light chain variable domain or a pseudo-light chain variable domain and a light chain constant domain, and component B comprises a parental masking moiety. In other embodiments, the QZ probe comprises an activatable pseudo-antibody, wherein the antibody component of the activatable pseudo-antibody is a variant of a parental antibody, wherein the bipartite polypeptide is a heavy chain, wherein component A comprises a heavy chain variable domain or a pseudo-heavy chain variable domain, a CH1, a CH2, a CH3, a hinge region, and an Fc, and component B comprises a parental masking moiety. In certain of these embodiments, the bipartite polypeptide has the structure of, from N-terminus to C-terminus, B-CL-A.

In some embodiments, the QZ probe comprises an activatable pseudo-antibody, wherein the first and second component B of the above-described bipartite polypeptides each independently comprise a parental masking moiety. In some aspects, the QZ probe comprises an activatable pseudo-antibody comprising a first polypeptide, a second polypeptide, a third polypeptide, and a fourth polypeptide, wherein:
the first polypeptide is a first light chain comprising a first bipartite polypeptide, wherein component A comprises (i) a first domain selected from the group consisting of first pseudo-light chain variable domain and a first light chain variable domain, and (ii) a light chain constant domain, and component B comprises a first parental masking moiety;
the second polypeptide is a second light chain comprising a second bipartite polypeptide, wherein component A comprises (i) a second domain selected from the group consisting of a second pseudo-light chain variable domain and a second light chain variable domain, and (ii) a light chain constant domain, and component B comprises a second parental masking moiety;
the third polypeptide is a heavy chain comprising (i) a third domain selected from the group consisting of a first pseudo-heavy chain domain and a first heavy chain variable domain, and (ii) a CH1, a CH2, a CH3, a hinge region, and an Fc domain; and
the fourth polypeptide is a heavy chain comprising (i) fourth domain selected from the group consisting of a second pseudo-heavy chain domain and a second heavy chain variable domain, and (ii) a CH1, a CH2, a CH3, a hinge region, and an Fc domain, with the proviso that at least one of the first domain, the second domain, the third domain, and the fourth domain is a pseudo-variable domain. In some of these embodiments the first component A comprises a light chain variable domain and the second component A comprises a light chain variable domain. In other embodiments, the first component A comprises a pseudo-light chain variable domain and the second component A comprises a pseudo-light chain variable domain. In some of the above-described embodiments, the bipartite polypeptides have the structure of, from N-terminus to C-terminus, B-CL-A.

In other aspects, QZ probe comprises an activatable pseudo-antibody comprising a first polypeptide, a second polypeptide, a third polypeptide, and a fourth polypeptide, wherein:
the first polypeptide is a first light chain comprising (i) a domain selected from the group consisting of first pseudo-light chain variable domain and a first light chain variable domain, and (ii) a light chain constant domain;
the second polypeptide is a second light chain comprising (i) a domain selected from the group consisting of a second pseudo-light chain variable domain and a second light chain variable domain, and (ii) a light chain constant domain;
the third polypeptide is a heavy chain comprising a first bipartite polypeptide, wherein component A comprises (i) a domain selected from the group consisting of a first pseudo-heavy chain variable domain and a first heavy chain variable domain and (ii) a CH1, a CH2, a CH3, a hinge region, and an Fc domain, and component B comprises a first parental masking moiety; and
the fourth polypeptide is a heavy chain comprising a bipartite polypeptide, wherein component A comprises (i) a domain selected from the group consisting of a second pseudo-heavy chain domain and a second heavy chain variable domain, and (ii) a CH1, a CH2, a CH3, a hinge region, and an Fc domain, and component B comprises a second parental masking moiety, with the proviso that at least one of the first domain, the second domain, the third domain, and the fourth domain is a pseudo-antibody variable domain. In some of these embodiments the first component A comprises a heavy chain variable domain and the second component A comprises a heavy chain variable domain. In other embodiments, the first component A comprises a pseudo-heavy chain variable domain and the second component A comprises a pseudo-heavy chain variable domain. In some of the above-described embodiments, the bipartite polypeptides have the structure of, from N-terminus to C-terminus, B-CL-A.

In certain of the above-described embodiments in which the QZ probe comprises a complex of four polypeptides, the first polypeptide and the second polypeptide comprise the same amino acid sequence. In other embodiments, the first polypeptide and the second polypeptide comprise different sequences. In other embodiments, the third polypeptide and the fourth polypeptide have the same amino acid sequence. In some embodiments, the third polypeptide and the fourth polypeptide have different amino acid sequences.

The CL of the bipartite polypeptides employed herein may comprise any of a variety of protease substrates. Suitable substrates include any that are known in the art, or that may be identified using any of a variety of known techniques including those described in U.S. Pat. Nos. 7,666,817, 8,563,269, PCT Publication No. WO 2014/026136, and Boulware et al. “Evolutionary optimization of peptide substrates for proteases that exhibit rapid hydrolysis kinetics,” Biotechnol Bioeng. (2010) 106.3: 339-46, each of which is incorporated by reference in their entireties.

In some embodiments, the CL comprises a substrate for a protease that is active, e.g., upregulated or otherwise unregulated, in a disease condition or diseased tissue. Exemplary disease conditions include, for example, a cancer (e.g., where the diseased tissue is a tumor tissue) and an inflammatory or autoimmune condition (e.g., where the diseased tissue is inflamed tissue). In some embodiments, the CL comprises a substrate for an extracellular protease. In other embodiments, the CL comprises a substrate for an intracellular protease.

Exemplary substrates include those that are substrates for any one or more of the following proteases: a disintegrin and metalloprotease (ADAM), an ADAM-like, or a disintegrin and metalloproteinase with thrombospondin motifs (ADAMTS, such as, for example, ADAM8, ADAM9, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example, Caspase 1, Caspase 2, Caspase 3, Caspase 4, Caspase 5, Caspase 6, Caspase 7, Caspase 8, Caspase 9, Caspase 10, Caspase 14, and the like); a cysteine cathepsin (such as, for example, Cathepsin B, Cathepsin C, Cathepsin K, Cathepsin L, Cathepsin S, Cathepsin V/L2, Cathepsin X/Z/P); a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metalloproteinase (such as, for example, Meprin, Neprilysin, prostate-specific membrane antigen (PSMA), bone morphogenetic protein 1 (BMP-1), and the like); a matrix metalloproteinase (MMP, such as, for example, MMP1, MMP2, MMP3, MMP7, MMP8, MMP9, MMP10, MMP11, MMP12, MMP13, MMP14, MMPP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like); elastase, granzyme B, Guanidinobenzoatase, HtrA1, proteinase 3, neutrophil elastase, neutrophil serine protease 4 (NSP4), Lactoferrin, Marapsin, NS3/4A, PACE4, Plasmin, prostate-specific antigen (PSA), tissue plasminogen activator (tPA), Thrombin, Tryptase, urokinase-type plasminogen activator (uPA), a Type II transmembrane Serine Protease (TTSP) (such as, for example, DESC1, DPP-4, FAP, Hepsin, Matriptase-2, MT-SP1/Matriptase, TMPRSS2, TMPRSS3, TMPRSS4, TMPRSS5, TMPRSS6, TMPRSS7, TMPRSS8, TMPRSS9, TMPRSS10, TMPRSS11, and the like), and the like. Specific substrates are described, for example, in WO 2010/081173, WO 2015/048329, WO 2015/116933, and WO 2016/118629, each of which is incorporated herein by reference in its entirety.

In certain embodiments, the CL comprises a substrate for at least one protease selected from the group consisting of a matrix metalloprotease (MMP), such as MMP2, thrombin, an aspartate protease, a cysteine protease (e.g., a cathepsin), threonine protease, legumain, and a serine protease, such as matriptase (MT-SP1), and urokinase (uPA).

In some embodiments, the CL comprises a substrate for at least one MMP. In certain of these embodiments, the MMP is selected from the group consisting of MMP1, MMP2, MMP3, MMP9, MMP11, MMP13, MMP14, MMPP17, and MMP19. In a specific embodiment, the CL comprises a substrate for MMP2. In certain specific embodiments, the CL comprises a substrate for MMP9.

The CL (and substrate therein) employed in the design of the QZ probe may be selected based on apriori knowledge of specific proteases suspected of being active in the biological sample of interest. In certain embodiments, a CL is selected from the group consisting of SEQ ID NOs: 17-84.

The specific combination of assay conditions may vary because the assay is tolerant to a wide range of conditions. During the contacting step, the QZ probe may be utilized in the form of an aqueous solution. The solution may further comprise one or more buffering agents or buffers to maintain the pH of the QZ probe solution at a desired pH. Buffers that are suitable for use in the QZ probe solution include any buffer that will not degrade the physical integrity of the biological sample. In some instances, the buffer is one that maintains pH in the range of from about 2.0 to about 9.0. In some instances, the buffer is one that maintains pH in the range of from about 5.0 to about 8.5. In some instances, the buffer is one that maintains pH in the range of from about 7.0 to about 8.0. An exemplary buffer that is suitable for use in the practice of the present invention comprises Tris hydrochloride, Calcium dichloride, Zinc chloride, and Tween®-20 (polysorbate 20). Other exemplary buffers that are suitable for use according to the present disclosure include Phosphate-buffered saline (PBS), N-2-hydroxyethylpiperazine-N′-2-ethanesulfonic acid (HEPES) buffer, and the like.

The QZ probe solution typically has a pH in the range of from about 2.0 to about 9.0, or sometimes in the range of from about 5.0 to about 8.5, and often in the range of from about 7.0 to about 8.0.

When the biological sample comprises a solid biological sample, e.g., a tissue sample, a cell sample, and the like, the resulting mixture comprises both a solid and a liquid. After incubating the mixture comprising the solid and the liquid for a suitable time, the liquid (or a liquid sample) is separated from the solid in the incubated mixture and the measuring step is conducted on the liquid (or liquid sample) to assess protease activity. When the biological sample comprises a liquid, e.g., a plasma sample, and the like, the measuring step can be conducted by measuring the incubated liquid or a sample thereof to assess protease activity.

Suitable biological samples include those that have been previously frozen. In some instances, the biological sample is frozen within about 6 hours post excision or extraction from a mammal. In some instances, the biological sample is frozen within about 2 hours post excision or extraction. In some instances, the biological sample is frozen within about 1 hour post excision or extraction. In some instances, the biological sample is frozen within about 30 minutes post excision or extraction. In some instances, the biological sample is frozen immediately after excising or extracting from a mammal.

In some embodiments, biological samples are employed that have been previously frozen at any temperature below 0° C. In some instances, the biological sample has been previously frozen at a temperature in the range of from about 0° C. to about −200° C., or in the range of from about −10° C. to about −100° C., or in the range of from about −20° C. to about −80° C.

In some instances, the frozen biological sample is staged at room temperature (e.g., a temperature in the range of from about 20 to about 25° C.) for at least about 30 seconds, or at least about 1 minute, or at least about 30 minutes, or at least about 6 hours, or about 24 hours prior to contact with the QZ probe. Often the biological sample is prepared under an ambient conditions (i.e., ambient temperature, pressure, and humidity).

When deployed as an aqueous solution (i.e. the “QZ probe solution”), the concentration of QZ probe in the QZ probe solution may be in the range of from about 0.1 μg/mL to about 500 μg/mL, or in the range of from about 1 μg/mL to about 100 μg/mL, or in the range of from about 5 μg/mL to about 50 μg/mL. The mixture volume may be in the range of from about 1 μL to about 2000 μL, or in the range of from about 10 μL to about 1000 μL, or in the range of from about 100 μL to about 500 μL.

As described hereinabove, in some instances, the biological sample is a tissue sample. In some embodiments, the tissue sample has a thickness in the range of from about 1 μm to about 250 μm, or in the range of from about 5 μm to about 100 μm, or in the range of from about 10 μm to about 50 μm. In some instances, the tissue sample has a surface area in the range of from about 1 mm² to about 1000 mm², or in the range of from about 10 mm² to about 500 mm², or in the range of from about 20 mm² to about 100 mm². In some instances, the ratio of mixture volume to the tissue sample surface area is in the range of from about 0.001 μL/mm² to about 2000 μL/mm², or in the range of from about 0.01 μL/mm² to about 1000 μL/mm², or in the range of from about 0.1 μL/mm² to about 100 μL/mm², or in the range of from about 1 μL/mm² to about 10 μL/mm².

During the incubation step, the mixture of biological sample and QZ probe is incubated for a period of time and at a temperature sufficient to allow any proteases present in the biological sample sufficient time to cleave the CL of the bipartite probe. In some embodiments, the mixture is incubated for a suitable time at a temperature in the range from about 4° C. to about 42° C., or in the range from about 10° C. to about 40° C., or in the range from about 20° C. to about 37° C. A suitable time can be in the range of from about 5 minutes to about 168 hours, or in the range of from about 1 hour to about 60 hours, or in the range of from about 24 hours to about 48 hours.

In some embodiments, the QZ probe further comprises a detectable label to facilitate detection of the bipartite polypeptide and cleaved polypeptides. In certain embodiments, a labelled secondary reagent is employed to facilitate detection, such as a labelled secondary antibody that binds to the bipartite polypeptide.

Any of a variety of moieties may be used as a label. Suitable labels include, for example, an imaging agent (such as, for example, a radioisotope (e.g., indium, technetium, ¹²⁵I, ¹³³Xe, a contrasting agent (such as, for example, iodine, gadolinium, iron oxide, and the like), an enzyme (such as, for example, horseradish peroxidase, alkaline phosphatase, β-galactosidase, and the like), a fluorescent label (such as, for example, yellow fluorescent protein (YFP), cyan fluorescent protein (CFP), green fluorescent protein (GFP), modified red fluorescent protein (mRFP), red fluorescent protein dimer2 (RFP tdimer2), HCRED, a europium derivative, and the like; a bioluminescent label, such as D-luciferin, and the like), a luminescent label (such as, for example an N-methylacrydium derivative, and the like) a dye, one or more metal ions, a ligand-based label (such as, for example, biotin, avidin, streptavidin, a hapten, and the like); and the like.

In some embodiments, the detectable label comprises a fluorescent label that has an absorption wavelength in the range of from 400 nm to 900 nm. In some embodiments, the detectable label comprises an Alexa Fluor® label, such as Alex Fluor® 647 or Alexa Fluor® 750. In some embodiments, the detectable label is attached to the bipartite polypeptide with a degree of labeling (DOL) in the range of from about 1.0 to about 5.0, or in the range of from about 2.0 to about 3.5. As used herein, the terms “Degree of Labeling”, “DOL”, “Degree of Substitution”, or “DOS” can be used interchangeably to refer to a molar ratio of label to polypeptide.

The methods of the present disclosure may be useful as a translational screen for identifying patients who might be responsive to a particular protease-activatable therapeutic molecule (see Example 5). As such, the present disclosure further provides a method of identifying a patient suitable for a treatment with a protease-activatable therapeutic molecule, the method comprising:
determining the level of protease activity in a biological sample from a patient according to any of the methods described herein,
   wherein the protease-activatable therapeutic molecule is activated by a target protease, and
   wherein, if the biological sample is determined to have target-protease activity, then the patient is identified as being suitable for treatment with a protease-activatable therapeutic molecule.

As used herein, the term “patient” refers to a mammal. Typically, the patient is a human. In a further embodiment, the present disclosure provides a method of treating a patient having a disease or disorder with a protease-activatable therapeutic molecule that is activated by a target protease, the method comprising:
administering to a patient having a disease or disorder a therapeutically effective amount of a protease-activatable therapeutic molecule,
wherein the patient has been identified as suitable for treatment with the protease-activatable therapeutic molecule in accordance with the methods for identifying a patient suitable for treatment with the protease-activatable therapeutic molecule, as described hereinabove.

Patients identified as being suitable for treatment with a particular protease-activatable therapeutic molecule may be afflicted with a variety of disorders or diseases. Exemplary disorders and diseases include, for example, a cardiovascular disease, a neoplastic disease, a neurodegenerative disease, an inflammatory disease, a skin disease, an infectious disease, a bacterial infection, a viral infection, an autoimmune disease, a metabolic disease, a hematologic disease, a cancer, and the like. Potential protease-activatable therapeutic molecules include protease-activatable therapeutic polypeptides and polypeptide complexes. Exemplary protease-activatable therapeutic molecules include, for example, protease-activatable antibodies, and other polypeptides, including, for example, cytokines, and the like.

Examples of methods and compositions also include those described in Howng B et al., “Novel Ex Vivo Zymography Approach for Assessment of Protease Activity in Tissues with Activatable Antibodies” Pharmaceutics. 2021 Sep. 2; 13(9):1390, which is incorporated by reference herein in its entirety.

The present application also provides aspects and embodiments as set forth in the following numbered Statements:

Statement 1. A method of determining the level of protease activity in a biological sample, the method comprising:
(a) contacting a biological sample with a solution comprising a QZ probe to form a mixture, wherein the QZ probe comprises at least one bipartite polypeptide having a component A, a cleavable linker (CL), and a component B in a structural arrangement of, from N-terminus to C-terminus,

A-CL-B or B-CL-A,
wherein the component A and the component B are each independently a polypeptide,
wherein CL comprises a substrate for a protease,
wherein cleavage of the CL generates a cleavage product comprising a cleaved polypeptide comprising the component A or a portion thereof, and a cleaved polypeptide comprising the component B or a portion thereof,
(b) incubating the mixture, thereby forming an incubated mixture comprising an incubated liquid; and
(c) measuring the quantity of one or more analytes in a sample of the incubated liquid to determine the level of protease activity in the biological sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

Statement 2. The method of claim 1, wherein the biological sample is a cell.

Statement 3. The method of claim 1, wherein the biological sample is an organoid.

Statement 4. A method of determining the level of protease activity in a biological sample, the method comprising:
(a) contacting a biological sample with a solution comprising a QZ probe to form a mixture, wherein the biological sample is a tissue sample, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide that comprises component A or a portion thereof, and a cleaved polypeptide comprising component B or a portion thereof,
(b) incubating the mixture, thereby forming an incubated mixture comprising the tissue sample and an incubated liquid; and
(c) measuring the quantity of one or more analytes in a sample of the incubated liquid to determine the level of protease activity in the tissue sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

Statement 5. A method of determining the level of protease activity in a biological sample comprising a liquid, the method comprising:
(a) contacting a biological sample with a QZ probe to form a mixture, wherein the biological sample comprises a liquid, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide comprising one or more cleaved polypeptide species comprising the component A or a portion thereof and a cleaved polypeptide comprising the component B or a portion thereof,
(b) incubating the mixture thereby forming an incubated mixture comprising an incubated liquid; and
(c) measuring the quantity of one or more analytes in a sample of incubated liquid to determine the level of protease activity in the biological sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

Statement 6. The method of Statement 5, wherein the biological sample is selected from the group consisting of a cell culture supernatant, a cell lysate supernatant, an organoid culture supernatant, blood, bile, bone marrow aspirate, breast milk, cerebrospinal fluid, plasma, saliva, serum, sputum, synovial fluid, and urine.

Statement 7. The method of Statement 5, wherein the biological sample is plasma.

Statement 8. The method of any of Statements 1-7, wherein the bipartite polypeptide comprises the structure of, from N-terminus to C-terminus, B-CL-A.

Statement 9. The method of any of Statements 1-8, wherein the QZ probe comprises a polypeptide complex comprising one or more further polypeptides.

Statement 10. The method of any of Statements 1-9, wherein the QZ probe comprises an antibody, wherein at least one of the components A and B of the bipartite polypeptide comprises an antibody domain selected from the group consisting of a light chain variable domain, a heavy chain variable domain, and a combination thereof.

Statement 11. The method of Statement 10, wherein the QZ probe comprises an antibody comprising a first polypeptide, a second polypeptide, a third polypeptide, and a fourth polypeptide, wherein:
(1) the first polypeptide comprises a bipartite polypeptide comprising a first component A that comprises a first light chain variable domain;
(2) the second polypeptide comprises a second component A comprising a second light chain variable domain;
(3) the third polypeptide comprises a first heavy chain variable domain; and
(4) the fourth polypeptide comprises a second heavy chain variable domain.

Statement 12. The method of Statement 10 or 11, wherein the QZ probe comprises an antibody comprising a first polypeptide, a second polypeptide, a third polypeptide, and a fourth polypeptide, wherein: (1) the first polypeptide comprises a bipartite polypeptide comprising a first component A that comprises a first heavy chain variable domain, (2) the second polypeptide comprises a second component A comprising a second heavy chain variable domain; (3) the third polypeptide comprises a first light chain variable domain; and (4) the fourth polypeptide comprises a second light chain variable domain.

Statement 13. The method of any of Statements 1-12, wherein each component B comprises a polypeptide having at least 3 amino acid residues.

Statement 14. The method of any of Statements 1-13, wherein the QZ probe comprises an activatable antibody and each component B comprises a masking moiety.

Statement 15. The method of Statement 14, wherein the QZ probe comprises an activatable antibody comprising a first, a second, a third, and a fourth polypeptide, wherein:
the first polypeptide is a first light chain comprising a first bipartite polypeptide, wherein the component A comprises a first light chain variable domain, and a light chain constant domain, and the component B comprises a first masking moiety;
the second polypeptide is a second light chain comprising a second bipartite polypeptide, wherein the component A comprises a second light chain variable domain, and a light chain constant domain, and the component B comprises a second masking moiety;
the third polypeptide is a first heavy chain comprising a first heavy chain variable domain, and a CH1, a CH2, a CH3, a hinge region, and an Fc domain; and the fourth polypeptide is a second heavy chain comprising a second heavy chain variable domain, and a CH1, a CH2, a CH3, a hinge region, and an Fc domain.

Statement 16. The method of Statement 14, wherein the QZ probe comprises an activatable antibody comprising a first, a second, a third, and a fourth polypeptide, wherein:
the first polypeptide is a first light chain comprising a first light chain variable domain, and a light chain constant domain;
the second polypeptide is a second light chain comprising a second light chain variable domain and a light chain constant domain;
the third polypeptide is a first heavy chain comprising a first bipartite polypeptide,
wherein the component A comprises a first heavy chain variable domain and a CH1, a CH2, a CH3, a hinge region, and an Fc domain, and the component B comprises a first masking moiety; and
the fourth polypeptide is a second heavy chain comprising a bipartite polypeptide,
wherein the component A comprises a second heavy chain variable domain, and a CH1, a CH2, a CH3, a hinge region, and an Fc domain, and the component B comprises a second masking moiety.

Statement 17. The method of any of Statements 1-9, wherein the QZ probe comprises a pseudo-antibody, wherein one of the component A and the component B comprises a pseudo-antibody variable domain selected from the group consisting of a pseudo-light chain variable domain and a pseudo-heavy chain variable domain, and a combination thereof.

Statement 18. The method of any of Statements 1-9, wherein the QZ probe comprises a pseudo-antibody comprising the bipartite polypeptide and a second polypeptide, wherein either
(i) the component A comprises a pseudo-light chain variable domain and the second polypeptide comprises a domain selected from the group consisting of a heavy chain variable domain and a pseudo-heavy chain variable domain; or
(ii) the component A comprises a pseudo-heavy chain variable domain and the second polypeptide comprises a domain selected from the group consisting of a light chain variable domain and a pseudo-light chain variable domain.

Statement 19. The method of any of Statements 1-9, wherein the QZ probe comprises a pseudo-antibody comprising the bipartite polypeptide and a second polypeptide, wherein either
(i) the component A comprises a light chain variable domain and the second polypeptide comprises a pseudo-heavy chain domain; or
(ii) the component A comprises a heavy chain variable domain and the second polypeptide comprises a pseudo-light chain variable domain.

Statement 20. The method of any of Statements 1-9, wherein the QZ probe comprises a pseudo-antibody comprising a first polypeptide, a second polypeptide, a third polypeptide, and a fourth polypeptide, wherein:
(1) the first polypeptide is a light chain comprising a bipartite polypeptide, wherein the component A comprises a domain selected from the group consisting of a first light chain variable domain and a first pseudo-light chain variable domain;
(2) the second polypeptide is a light chain comprising a bipartite polypeptide, wherein the component A comprises a domain selected from the group consisting of a second light chain variable domain and a second pseudo-light chain variable domain;
(3) the third polypeptide is a heavy chain comprising a first pseudo-heavy chain variable domain; and
(4) the fourth polypeptide is a heavy chain comprising a second pseudo-heavy chain variable domain.

Statement 21. The method of any of Statements 1-9, wherein the QZ probe comprises a pseudo-antibody comprising a first polypeptide, a second polypeptide, a third polypeptide, and a fourth polypeptide, wherein (1) the first polypeptide is a light chain comprising a first bipartite polypeptide, wherein the component A comprises a first pseudo-light chain variable domain; (2) the second polypeptide is a light chain comprising a second bipartite polypeptide, wherein the component A comprises a second pseudo-light chain variable domain; (3) the third polypeptide is a heavy chain comprising a domain selected from the group consisting of a first heavy chain variable domain and a first pseudo-heavy chain variable domain; and (4) the fourth polypeptide is a heavy chain comprising a domain selected from the group consisting of a second heavy chain variable domain and a second pseudo-heavy chain variable domain.

Statement 22. The method of any of Statements 17-21, wherein each component B is a polypeptide comprising at least about 3 amino acid residues.

Statement 23. The method of any of Statements 17-21, wherein the pseudo-antibody is a variant of a parental antibody and wherein each component B comprises a parental masking moiety.

Statement 24. The method of any of Statements 1-9, wherein the QZ probe comprises an activatable pseudo-antibody comprising a first polypeptide, a second polypeptide, a third polypeptide, and a fourth polypeptide,
wherein:
the first polypeptide is a first light chain comprising a first bipartite polypeptide,
wherein the component A comprises (i) a first domain selected from the group consisting of first pseudo-light chain variable domain and a first light chain variable domain, and (ii) a light chain constant domain, and the component B comprises a first parental masking moiety;
the second polypeptide is a second light chain comprising a second bipartite polypeptide, wherein the component A comprises (i) a second domain selected from the group consisting of a second pseudo-light chain variable domain and a second light chain variable domain, and (ii) a light chain constant domain, and the component B comprises a second parental masking moiety;
the third polypeptide is a heavy chain comprising (i) a third domain selected from the group consisting of a first pseudo-heavy chain domain and a first heavy chain variable domain, and (ii) a CH1, a CH2, a CH3, a hinge region, and an Fc domain; and
the fourth polypeptide is a heavy chain comprising (i) fourth domain selected from the group consisting of a second pseudo-heavy chain domain and a second heavy chain variable domain, and (ii) a CH1, a CH2, a CH3, a hinge region, and an Fc domain, with the proviso that at least one of the first domain, the second domain, the third domain, and the fourth domain is a pseudo-variable domain.

Statement 25. The method of any of Statements 1-9, wherein the QZ comprises an activatable pseudo-antibody comprising a first polypeptide, a second polypeptide, a third polypeptide, and a fourth polypeptide, wherein:
the first polypeptide is a first light chain comprising (i) a domain selected from the group consisting of first pseudo-light chain variable domain and a first light chain variable domain, and (ii) a light chain constant domain;
the second polypeptide is a second light chain comprising (i) a domain selected from the group consisting of a second pseudo-light chain variable domain and a second light chain variable domain, and (ii) a light chain constant domain;
the third polypeptide is a heavy chain comprising a first bipartite polypeptide, wherein the component A comprises (i) a domain selected from the group consisting of a first pseudo-heavy chain variable domain and a first heavy chain variable domain and (ii) a CH1, a CH2, a CH3, a hinge region, and an Fc domain, and the component B comprises a first parental masking moiety; and
the fourth polypeptide is a heavy chain comprising a bipartite polypeptide, wherein the component A comprises (i) a domain selected from the group consisting of a second pseudo-heavy chain domain and a second heavy chain variable domain, and (ii) a CH1, a CH2, a CH3, a hinge region, and an Fc domain, and the component B comprises a second parental masking moiety,
   with the proviso that at least one of the first domain, the second domain, the third domain, and the fourth domain is a pseudo-antibody variable domain.

Statement 26. The method of any of Statements 1-25, further comprising performing a plurality of cycles of steps (a)-(c).

Statement 27. The method of Statement 26, wherein the CL in each of the plurality of cycles or subset thereof, is different.

Statement 28. The method of any of Statements 26-27, wherein the component A in each of the plurality of cycles or subset thereof, is the same.

Statement 29. The method of any of Statements 26-27, wherein the component B in each of the plurality of cycles or subset thereof, is the same.

Statement 30. The method of any of Statements 26-27, wherein the component A in each of the plurality of cycles or subset thereof, is the same, and the component B in each of the plurality of cycles or subset thereof, is the same.

Statement 31. The method of any of Statements 1-30, further comprising performing a plurality of cycles of steps (a)-(c), wherein in each cycle, the biological sample is incubated with one or more protease inhibitors or a combination of two or more protease inhibitors prior to step (a) and/or during step (a).

Statement 32. The method of Statement 31, wherein the protease inhibitor in each cycle of the plurality of cycles is different.

Statement 33. The method of any of Statements 31-32, wherein the QZ probe in each cycle of the plurality of cycles is the same.

Statement 34. The method of any of Statements 26-33, wherein the plurality of cycles is performed in parallel.

Statement 35. The method of Statement 34, wherein the plurality of cycles is performed in a multi-well plate.

Statement 36. The method of any of Statements 26-35, wherein the plurality of cycles is performed in series.

Statement 37. The method of any of Statements 1-36, wherein the QZ probe comprises a plurality of distinct species of QZ probes, and wherein the biological sample is contacted with the plurality of distinct QZ probes.

Statement 38. The method of Statement 37, wherein each distinct species of QZ probe in the plurality or subset thereof comprises a CL having a different substrate.

Statement 39. The method of any of Statements 37-38, wherein measuring the quantity of one or more analytes in a sample of the incubated liquid comprises measuring a distinct signal associated with each distinct species of QZ probe in the plurality.

Statement 40. The method of any of Statements 1-39, wherein each CL comprises a substrate for a protease selected from the group consisting of a disintegrin and metalloprotease (ADAM), a disintegrin and metalloproteinase with thrombospondin motifs (ADAMTS), an aspartate protease, an aspartic cathepsin, a caspase, a cysteine cathepsin, a cysteine proteinase, a KLK, a metalloproteinase, a matrix metalloproteinase (MMP), a serine protease, a coagulation factor protease, and a Type II Transmembrane Serine Protease (TTSP).

Statement 41. The method of any of Statements 1-39, wherein each CL comprises a substrate for at least one protease selected from the group consisting of ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathepsin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP1, MMP2, MMP3, MMP7, MMP8, MMP9, MMP10, MMP11, MMP12, MMPP13, MMP14, MMP15, MMP16, MMP17, MMP19, MMP20, MMP23, MMP24, MMP26, MMP27, activated protein C, Cathepsin A, Cathepsin G, Chymase, FVIIa, FIXa, FXa, FXI, FXIIa, elastase, granzyme B, Guanidinobenzoatase, HtrA1, proteinase 3, neutrophil elastase, NSP4, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matriptase, TMPRSS2, TMPRSS3, TMPRSS4, TMPRSS5, TMPRSS6, TMPRSS7, TMPRSS8, TMPRSS9, TMPRSS10, and TMPRSS11.

Statement 42. The method of any of Statements 1-39, wherein each CL comprises a substrate for a serine protease.

Statement 43. The method of any of Statements 1-39, wherein each CL comprises a substrate for a matrix metalloproteinase (MMP).

Statement 44. The method of any of Statements 1-39, each CL comprises a substrate for an aspartate protease, cysteine protease, or threonine protease.

Statement 45. The method of any of Statements 1-39, wherein each CL comprises a substrate having an amino acid sequence selected from the group consisting of SEQ ID NOs:17-84.

Statement 46. The method of any of Statements 1-45, wherein the one or more measured analytes comprise an uncleaved intact bipartite polypeptide.

Statement 47. The method of any of Statements 1-45, wherein the one or more measured analytes comprise one or more species of cleaved polypeptide comprising the component A or portion thereof and the uncleaved bipartite polypeptide.

Statement 48. The method of any of Statements 1-45, wherein the one or more measured analytes comprise one or more species of the cleaved polypeptide comprising the component B or portion thereof and the uncleaved bipartite polypeptide.

Statement 49. The method of any of Statements 1-45, wherein the one or more measured analytes comprise one or more species of the cleaved polypeptide comprising the component A or portion thereof, one or more species of the cleaved polypeptide comprising the component B or portion thereof, and the uncleaved bipartite polypeptide.

Statement 50. A method of identifying a patient suitable for a treatment with a protease-activatable therapeutic molecule, the method comprising:
determining the level of protease activity in a biological sample from a patient
according to the method of any of Statements 1-49,
wherein the protease-activatable therapeutic molecule is activated by a target protease, and
wherein, if the biological sample is determined to have target-protease activity, then the patient is identified as being suitable for treatment with a protease-activatable therapeutic molecule.

Statement 51. A method of treating a patient having a disease or disorder with a protease-activatable therapeutic molecule that is activated by a target protease, the method comprising:
administering to a patient having a disease or disorder a therapeutically effective amount of a protease-activatable therapeutic molecule,
wherein the patient has been identified as suitable for treatment with the protease-activatable therapeutic molecule in accordance with the method of Statement 50.

Statement 52. The method of any of Statements 50-51, wherein the patient has a disorder or a disease selected from the group consisting of a cardiovascular disease, a neoplastic disease, a neurodegenerative disease, an inflammatory disease, a skin disease, an infectious disease, a bacterial infection, a viral infection, an autoimmune disease, a metabolic disease, a hematologic disease, and a cancer.

Statement 53. The method of any of Statements 1-52, wherein the QZ probe comprises an activatable cytokine, wherein the component A of the bipartite polypeptide comprises a cytokine and the component B comprises a masking moiety.

Statement 54. The method of any of Statements 1-53, wherein each QZ probe further comprises a detectable label.

Statement 55. The method of any of Statements 1-54, further comprising measuring the quantity of analyte comprises using a secondary reagent that binds to at least one analyte, wherein the secondary reagent is attached to a detectable label.

Statement 56. The method of any of Statements 1-55, wherein the quantities of one or more analytes is/are determined by subjecting the sample(s) of incubated liquid to capillary electrophoresis.

Statement 57. The method of Statement 56, wherein the capillary electrophoresis is reducing capillary electrophoresis.

Statement 58. The method of any of Statements 1-57, wherein the quantities of one or more analyte is/are determined by subjecting the sample(s) of incubated liquid to a capillary electrophoresis immunoassay.

The invention of the present disclosure will be more readily understood by reference to the following examples, which are included merely for purposes of illustration of certain aspects and embodiments of the present invention, and are not intended to limit the scope of disclosed invention in any way.

### EXAMPLES

### Example 1

### Materials and Methods

Recombinant human MT-SP1 (3946-SEB), uPA (1310-SE), and MMP-2 (902-MP) were from R&D Systems. Human tumor samples were provided by the NCI Cooperative Human Tissue Network. The H292 cell line (CRL-1848) for xenograft studies was acquired from the American Type Culture Collection. Plasma samples were obtained from ProteoGenex.

cDNA coding for the polypeptides described herein were separately cloned into a modified pcDNA3.1 mammalian expression vector (Life Technologies). CHO-S cells (Life Technologies) were transiently transfected with the plasmids for 5-7 days using FreeStyle MAX transfection reagent (Life Technologies) following the manufacturer's instructions. Each was purified using a HiTrap Mab Select Sure Protein A column (GE Healthcare) coupled to an AKTA purifier (GE Healthcare). The purity and the homogeneity of each purified product was analyzed by SDS-PAGE in reducing and non-reducing conditions and size exclusion chromatography using a Superdex 200, 10/300 GL column (GE Healthcare), respectively.

Antibody C225, which is the anti-epidermal growth factor receptor (EGFR) antibody Cetuximab, was used as a parental antibody in this disclosure. Pseudo-antibody MC225 was generated by introducing several mutations in the heavy chain (HC) complementarity-determining regions (CDRs) of C225. The sequences of the parental C225 HC (SEQ ID NO: 1) and the mutant MC225_HC (SEQ ID NO: 3) are shown in Table 1, as the mutation areas are underlined in MC225_HC (SEQ ID NO: 3). Parental antibody C225 comprises SEQ ID NO:1 (heavy chain) and SEQ ID NO: 2 (light chain). Pseudo-antibody MC225 comprises SEQ ID NO: 3 (heavy chain with a pseudo-heavy chain variable domain) and SEQ ID NO: 4 (light chain). The parental light chain (SEQ ID NO: 2) is the same as the light chain employed in the pseudo-antibody (SEQ ID NO:4). The amino acid sequences are provided in Table 1.

TABLE 1
Sequences of Parental Antibody C225 and Pseudo-Antibody MC225.

| Name | Description | Sequence |
|---|---|---|
| C225_HC | Parental | QVQLKQSGPGLVQPSQSLSITCTVSGFSLTNYGVH |
| (SEQ ID NO: 1) | antibody C225 | WVRQSPGKGLEWLGVIWSGGNTDYNTPFTSRLSIN |
| | heavy chain | KDNSKSQVFFKMNSLQSQDTAIYYCARALTYYDYE |
| | | FAYWGQGTLVTVSAASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKRVEPKSCDKTHTCPPCPAPELLGGPSVFLFPPK |
| | | PKDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGK |
| C225_LC | Parental | QILLTQSPVILSVSPGERVSFSCRASQSIGTNIHWYQ |
| (SEQ ID NO: 2) | antibody C225 | QRTNGSPRLLIKYASESISGIPSRFSGSGSGTDFTLSI |
| | light chain | NSVESEDIADYYCQQNNNWPTTFGAGTKLELKRTV |
| | | AAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| MC225_HC | Pseudo-antibody | QVQLKQSGPGLVQPSQSLSITCTVSGFSLTSYGVHW |
| (SEQ ID NO: 3) | MC225 heavy | VRQSPGKGLEWLGVIWSKGSTAYNTPFTSRLSINKD |
| | chain | NSKSQVFFKMNSLQSQDTAIYYCARALTSGKAEFA |
| | | YWGQGTLVTVSAASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTKV |
| | | DKRVEPKSCDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGK |
| MC225_LC | Pseudo-antibody | QILLTQSPVILSVSPGERVSFSCRASQSIGTNIHWYQ |
| (SEQ ID NO: 4) | MC225 light | QRTNGSPRLLIKYASESISGIPSRFSGSGSGTDFTLSI |
| | chain | NSVESEDIADYYCQQNNNWPTTFGAGTKLELKRTV |
| | | AAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |

A series of activatable antibodies and activatable pseudo-antibodies were developed based on parental antibody C225 and pseudo-antibody MC225, respectively. Each heavy chain of activatable antibody comprises heavy chain C225_HC (SEQ ID NO: 1). Each heavy chain of activatable pseudo-antibody comprises pseudo-heavy chain MC225_HC (SEQ ID NO: 3). Each light chain of activatable antibody or activatable pseudo-antibody is a bipartite polypeptide comprising a (parental) masking moiety (component B), a cleavable linker and a C225 light chain variable domain and light chain constant domain (component A), in the orientation, from N-terminus to C-terminus, B-CL-A. The sequences of the light chains are listed in Table 2 (the masking moiety sequences (component B) are in bold and the cleavable linker (CL) sequences are underlined).

TABLE 2
Sequences of the Bipartite Polypeptide in an Activatable Pseudo-Antibody-
based QZ Probe

| NAME | SEQUENCE |
|---|---|
| C225-S01_LC | QGQSGQCISPRGCPDGPYVMYGSSGGSGGSGGSGLSGRSDNHGSSG |
| (SEQ ID NO: 5) | TQILLTQSPVILSVSPGERVSFSCRASQSIGTNIHWYQQRTNGSPRLLI |
| | KYASESISGIPSRFSGSGSGTDFTLSINSVESEDIADYYCQQNNNWPTT |
| | FGAGTKLELKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC |
| C225-M01_LC | QGQSGQCISPRGCPDGPYVMYGSSGGSGGSGGSGPLGLGSSGTQIL |
| (SEQ ID NO: 6) | LTQSPVILSVSPGERVSFSCRASQSIGTNIHWYQQRINGSPRLLIKYAS |
| | ESISGIPSRFSGSGSGTDFTLSINSVESEDIADYYCQQNNNWPTTFGAG |
| | TKLELKRTVAAPSVFIFPPSDEQLKSGTASVVCLLNNFYPREAKVQW |
| | KVDNALQSGNSQESVTEQDSKDSTYSLSSTLTLSKADYEKHKVYAC |
| | EVTHQGLSSPVTKSFNRGEC |
| MC225- | QGQSGQCISPRGCPDGPYVMYGSSGGSGGSGGSGISSGLLSGRSDN |
| Sub1_LC/C225- | HGSSGTQILLTQSPVILSVSPGERVSFSCRASQSIGTNIHWYQQRINGS |
| Sub1 LC | PRLLIKYASESISGIPSRFSGSGSGTDFTLSINSVESEDIADYYCQQNNN |
| (SEQ ID NO: 7) | WPTTFGAGTKLELKRTVAAPSVFIFPPSDEQLKSGTASVVCLLNNFYP |
| | REAKVQWKVDNALQSGNSQESVTEQDSKDSTYSLSSTLTLSKADYE |
| | KHKVYACEVTHQGLSSPVTKSFNRGEC |
| MC225- | QGQSGQCISPRGCPDGPYVMYGSSGGSGGSGGSGAVGLLAPPGGLS |
| Sub2_LC/C225- | GRSDNHGSSGTQILLTQSPVILSVSPGERVSFSCRASQSIGTNIHWYQQ |
| Sub1_LC | RTNGSPRLLIKYASESISGIPSRFSGSGSGTDFTLSINSVESEDIADYYC |
| (SEQ ID NO: 8) | QQNNNWPTTFGAGTKLELKRTVAAPSVFIFPPSDEQLKSGTASVVCL |
| | LNNFYPREAKVQWKVDNALQSGNSQESVTEQDSKDSTYSLSSTLTLS |
| | KADYEKHKVYACEVTHQGLSSPVTKSFNRGEC |

### EGFR Binding Assay (ELISA)

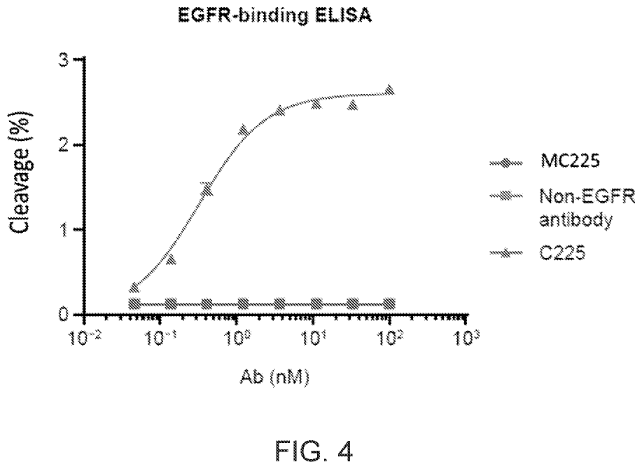

To confirm the pseudo-antibody MC225 was not capable of binding to the EGFR antigen, an EGFR binding assay was conducted. 96-well plates (Nunc) were coated with EGFR-Fc (50 ng/well; R&D Systems) in Hank's Balanced Salt Solution (HBSS pH 7.4, 10 mM Hepes) and blocked with HBSS containing 1% BSA. The plates were incubated with the indicated concentrations of parental antibody C225, pseudo-antibody MC225, or a non-EGFR antibody, in HBSS/1% BSA for 1 h at room temperature. The plates were then incubated with horseradish peroxidase (HRP) conjugated anti-human F(ab′)2 (Jackson ImmunoResearch Laboratories) in HBSS for 30 min. and the detection was performed by the addition of 3,3′,5,5′-tetramethylbenzidine substrate (1-Step Ultra-TMB, Pierce) followed by an equal volume of 1M hydrochloric acid. Absorbance at 450 nm was then measured and reported as optical density (OD 450 nm) as shown in FIG. 4. While C225 showed high-binding ability to immobilized EGFR, MC225 exhibited non-binding activity to the antigen, comparable to the negative control, the non-EGFR antibody.

### QZ Probe Labeling

QZ probes for assessment of tumor sections were conjugated with the far-red fluorescent Alexa Fluor® 647 dye (A20006, ThermoFisher Scientific) using N-hydroxysuccinimide (NHS) ester reaction. QZ probes were incubated with amine-reactive fluorescent dyes for 1 hour at room temperature, and the reaction was stopped with 10% (v/v) addition of 1 M tris(hydroxymethyl)aminomethane hydrochloride (Tris-HCl) buffer, pH 8.5. After conjugation, free dyes were removed using Zeba™ desalting columns (87768, ThermoFisher Scientific), according to manufacturer protocols. QZ probes with a degree of labeling of 2 to 3, as determined by a NanoDrop™ spectrophotometer (ThermoFisher Scientific), were used in most studies.

QZ probes for assessment of plasma samples were conjugated with Oregon Green dye (06149, ThermoFisher Scientific) using the same method.

### Tissue Samples

The protease activity in tumor sections was assessed using frozen tissue sections that had been stored at −80° C. for long-term storage and at −20° C. shortly before use. Slides were brought to room temperature and allowed to dry for 30 minutes before the assay. A hydrophobic barrier was drawn around the tissue sample to maintain liquid on the tissue using an ImmEdge™ Hydrophobic Barrier Pen (Vector Laboratories), and the slides were then incubated with buffer consisting of 150 mM Tris HCl pH 7.4, 5 mM CaCl₂) 100 μM ZnCl2, and 0.005% Tween®-20 (QZ assay buffer) for 30 minutes at room temperature. Labeled QZ probes prepared in QZ buffer were then added directly onto the tissue containing the buffer to form a mixture, and incubated at a concentration of 20 μg/mL in a humidified chamber at 37° C. for 48 hours.

For inhibitor treatment assays utilizing binding-competent QZ probes, tissue sections were blocked for 30 minutes with 3× unlabeled C225 antibody (60 μg/mL) and then preincubated for at least 30 minutes with 3× protease inhibitors or QZ buffer alone before addition of 3× labeled C225 QZ probe (60 μg/mL) in QZ buffer. Protease inhibitors and their final (1×) assay concentrations were as follows: 200 μg/mL aprotinin (78432, ThermoFisher Scientific), 10 μM Galardin or GM 6001 (364206, Calbiochem), and 1×EDTA or 1×HALT/EDTA Protease Inhibitor Cocktail (78438, ThermoFisher Scientific).

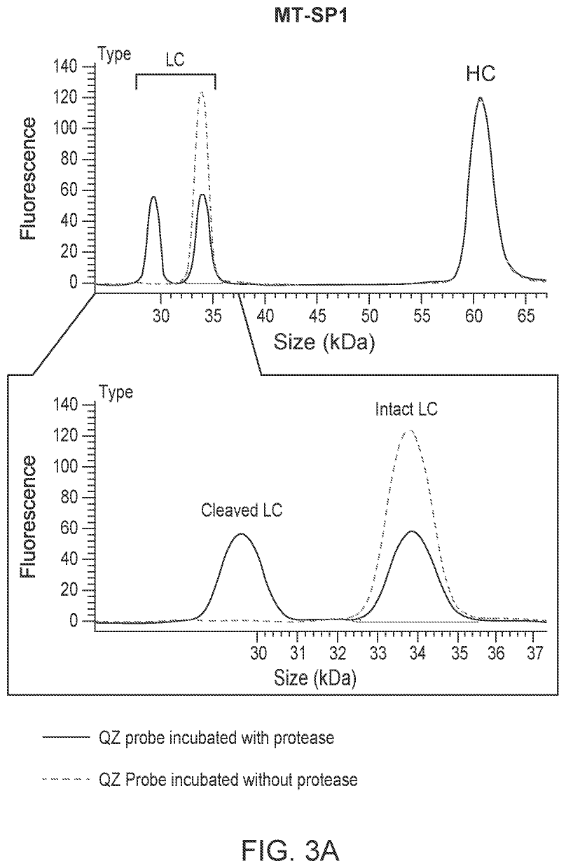

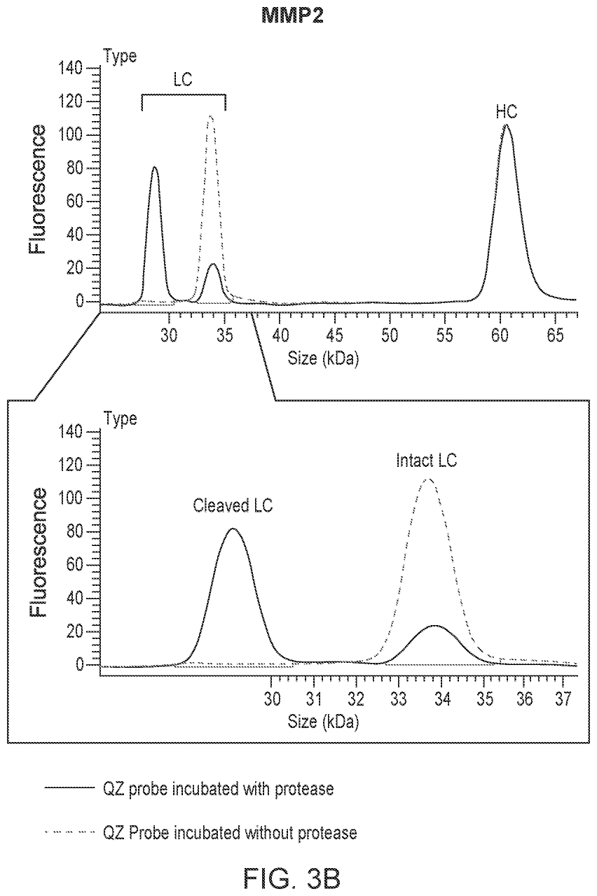

Following 48 hours of incubation, supernatant was collected from each incubated mixture and transferred into a well of a 96-well PCR plate for assay by reducing capillary electrophoresis. Each supernatant sample was mixed with Pico Sample Buffer (Perkin Elmer) containing 2-beta-mercaptoethanol at four parts sample and one part of Pico Sample Buffer and then heated at 95° C. for 10 minutes. The composition of each supernatant sample was then assessed using the Caliper LabChip GXII or LabChip GXII Touch (Caliper Life Sciences) with the HT Pico Protein Express 100 protocol (Perkin Elmer). Protein Express Assay LabChips (Perkin Elmer #760499) were set up using the protocol of the Protein Pico Assay Reagent Kit (Perkin Elmer #760498). The quantity of cleavage product present in each supernatant sample was calculated from the fluorescent signals of component cleavage products and intact light chains (i.e., intact bipartite probe) using the LabChip GX Reviewer software (Perkin Elmer) (e.g. for fluorescently labelled QZ probes). A schematic of this illustrative process is depicted in FIG. 2. Representative capillary electropherograms of QZ probe, C225-Sub1, incubated with human membrane type serine protease 1 (MT-SP1) or human matrix metalloproteinase-2 (MMP-2) are depicted in FIGS. 3A-3B.

### Plasma Samples

Oregon Green-labeled QZ probe in phosphate-buffered saline (PBS) pH 7.2 were added to an equal volume of plasma from healthy donors (n=5), or patients with lung cancer (n=5) or gastric cancer (n=5) (ProteoGenex), to give a final concentration of 714 μg/mL in a total volume of 10 μL. The QZ probe and plasma mixtures were incubated for 48 hours at 37° C. in a humidified chamber, then diluted 1:50 in PBS and analyzed by a capillary electrophoresis immunoassay (CEI). Samples were separated for 33 minutes to resolve component cleavage products and intact QZ probe components (i.e., including intact LC bipartite polypeptide), then incubated for 1 hour with goat polyclonal antibodies against Oregon Green Dye (1/40; A11095 ThermoFisher). Target proteins were immunoprobed using a 30-minute incubation with a horseradish peroxidase-conjugated secondary antibody (1/40; 705-035-147, Jackson ImmunoResearch) and chemiluminescent substrate (PS-CS0; ProteinSimple). Peaks with a signal-to-noise ratio of ≥5 were considered positive. This signal-to-noise ratio was calculated by the Simple Western Compass software (ProteinSimple).

### Illustrative Metrics for Characterizing Relative Level of Protease Activity

A convenient metric for relative protease activity is the percentage of a cleavage product (e.g., the cleaved light chain of MC225-Sub1 (the bipartite polypeptide)) in the incubated liquid using the following equations:

$$Protease activity ( \% ) = ( cleaved LC peak area ( s ) ) / \text{} ( cleaved LC peak area ( s ) + intact LC peak area ) \\ Protease activity ( \% ) = ( cleaved LC peak height ( s ) ) / ( cleaved LC height ( s ) + intact LC peak height ) .$$

The relative level of protease activity may also be characterized as the concentration of cleaved QZ probe using the following equation:

$$Cleaved QZ probe ( μg / mL ) = the initial QZ probe concentration \times protease activity ( \% ) as calculated above .$$

These are just examples of a number of different ways that levels of protease activity can be characterized according to the methods of the present disclosure.

k_{cat}/K_{M} Determination

Recombinant human uPA and MT-SP1 were incubated at 500 nM (uPA) and 2 μM (MT-SP1) concentrations with QZ probe in 50 mM Tris-HCl, pH 7.4, 150 mM NaCl, 0.05% Tween®-20, 5 mM calcium chloride for 24 h at 37° C. The reaction was stopped by adding 5 μl of sample to 7 μl of HT Protein Express Sample Buffer (Caliper LifeSciences) and incubating for 10 min at 95° C. Samples were analyzed by capillary electrophoresis (GXII; Caliper LifeSciences) and concentrations of component cleavage products and uncleaved bipartite polypeptide (i.e., intact light chain of the QZ probe) were determined using LabChip GX software (Caliper LifeSciences). The k_{cat}/K_{M} values were determined using the following equation:

$${k}_{cat} / {K}_{M} = - ln ( 1 - C ) / ( t \times p )$$

C=relative portion of product converted (cleaved LC peak area/(cleaved LC peak area+intact LC peak area), t=time (s) and p=protease concentration (M). The substrate concentration was maintained below the K_{M} and in excess of the protease.

### Example 2

### Optimization of Assay Conditions

QZ probes MC225-Sub1 and MC225-Sub2 containing protease substrates Sub1 or Sub2 were used to assess the effect of experimental variables on protease activity. Here, archival bladder cancer patient tumors, renal cell carcinoma (RCC) patient tumors, ovarian cancer patient tumors, or H292 human non-small cell lung cancer xenograft tumor sections were utilized.

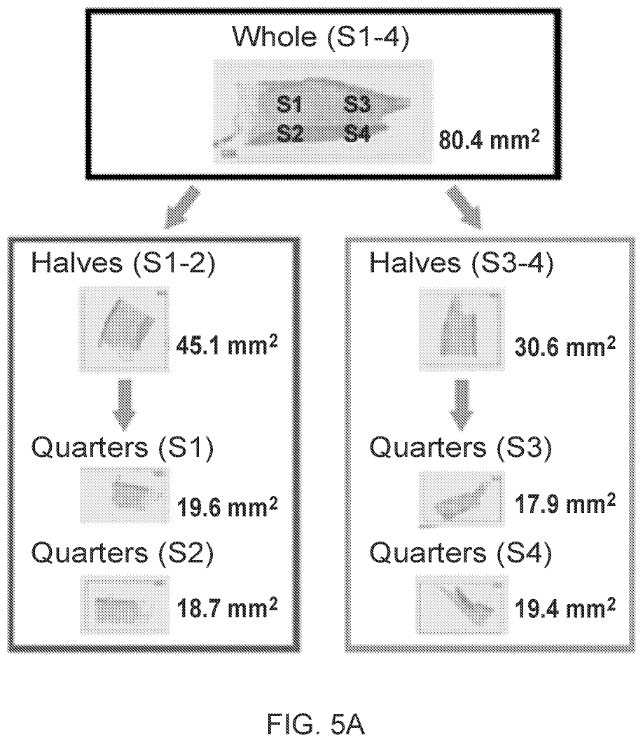

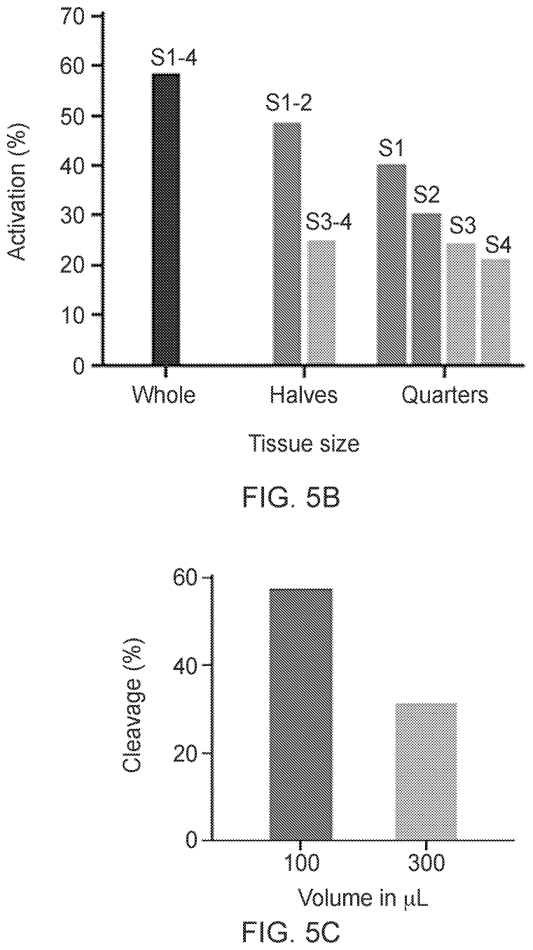

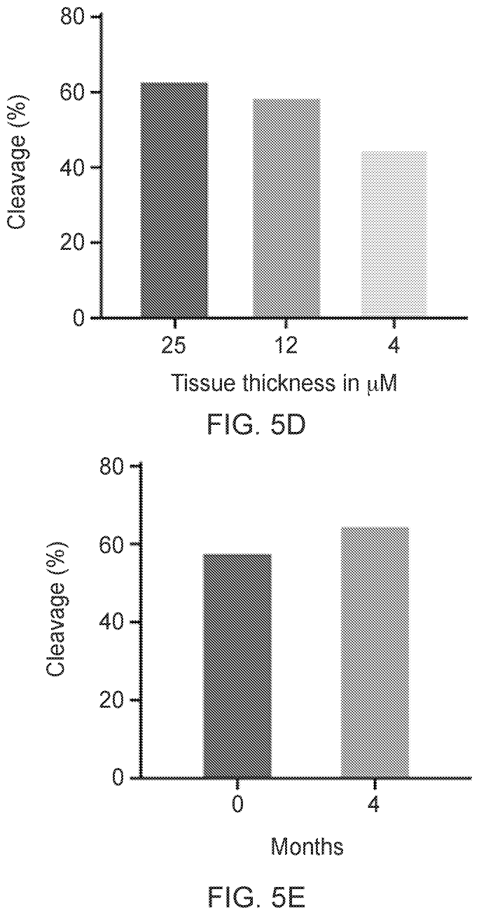

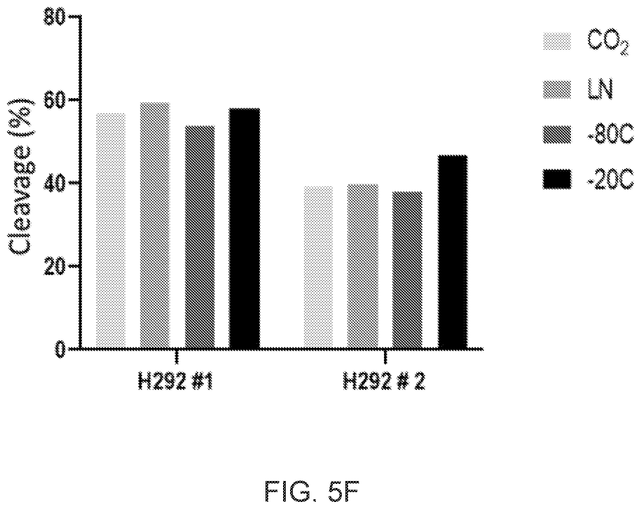

Tissue Size. Three human tumor serial sections were analyzed by leaving one whole (S1-4) and dividing the other two into halves (51-2 and S3-4) or quarters (S1, S2, S3, and S4) for tumor tissue correlation studies (FIG. 5A). The QZ assay was assessed at different section sizes using a constant assay volume (100 μL). As the tissue was halved and quartered, decreased activity was observed. The level of protease-cleaved QZ probe was found to be proportional to tissue content in the tissue halves and quarters (i.e., S1-4 vs S1-2 vs S1 and S2). However, some variabilities were also observed in the tissue quarters (i.e., S3-4 vs S3 and S4), indicating some degree of protease activity heterogeneity across the tumor sample (FIG. 5B).

Assay Volume. Protease activity assessment of tumor sections of the same size but with different assay buffer volumes (100 μL and 300 μL) were tested. The tumor section incubated with QZ probe in 100 μL buffer volume showed a higher protease activity, compared to the same size of the tumor section in 300 μL buffer volume (FIG. 5C). A correlation of QZ probe cleavage levels with the relative concentration of protease activity was observed.

Tissue Thickness. The effect of tumor section thickness in protease activity was also explored. Protease activities of a tissue sample were assessed at different section thicknesses, 25 μM, 12 μM, or 4 μM, in a constant assay volume (100 μL). The assay demonstrated that the QZ probe cleavage activity decreases with reduced section thickness (FIG. 5D).

Storage Time. Protease activity assays are performed on fresh or frozen tissues that maintain endogenous protease activity; therefore, conditions of tissue cryopreservation and storage were extensively evaluated. Specifically, assessment of protease activity in fresh compared to frozen and stored tumor tissue samples showed that QZ probe cleavage did not decrease with frozen storage at −80° C. for 1 month, 4 months, and up to 11 months (Table 3 and FIG. 5E). In this study, protease activity (%) more than about 30% is defined as high protease activity, protease activity between about 1% and about 30% is defined as low/medium protease activity, and protease activity less than 1% is defined as no protease activity (Neg). For subset of samples, two serial sections of the same sample with same storage condition was tested on the same time hence the reporting of two number in one box. This was not performed for all samples due to the scarcity of the tissues.

TABLE 3
Protease activity at −80° C. storage (tissue)

| | | Protease | Time |
|---|---|---|---|
| Sample ID | Indication | Activity (%) | at −80° C. |
| 131068 | Melanoma | High (32% and 39%) | 1 month |
| | | High/Medium (36% | 4 days |
| | | and 28%) | |
| 40846 | Bladder | High (57.52%) | 4 Months |
| | | High (64.38%) | 24 days |
| 40843 | Bladder | High (46%) | 8 Months |
| | | High (65.1%) | 9 days |
| 41924 | RCC | High (31.88%) | 10 Months |
| | | High (48.46% | 11 days |
| | | and 62.7%) | |
| 60362 | Ovarian | Neg (0) | 11 Months |
| | | Neg (0) | |
| 40834 | Bladder | Low/Medium (15.75%) | 9 Months |
| | | Low/Medium (10.91% | 3 days |
| | | and 13.1%) | |
| 40832 | Bladder | Low/Medium (14.49%) | 9 Months |
| | | Low/Medium (25% | 3 days |
| | | and 23.4%) | |

Freezing Process. Protease activity of tissue samples were tested by different freezing processes (liquid nitrogen or frozen carbon dioxide) or storage temperature (−80° C. or −20° C.). In FIG. 5F, two H292 samples were independently divided into fourths and frozen with different processes. Samples frozen using dry ice (C02), liquid nitrogen (LN) or −80° C. temperature were stored at −80° C. Samples frozen using −20° C. temperature were stored at −20° C. Protease activity was assessed on these different cryopreservation conditions. Activities were found to be similar regardless of freezing condition and storage temperature (FIG. 5F).

The incubation time was 24 hours for data shown in FIGS. 5B, 5C, and 5D, while the incubation time was 48 hours for data shown in FIGS. 5E and 5F.

Freeze Thaw. Protease activity was assessed on a series of tissue samples obtained from tissue sections that were subjected to freeze thaw cycles. The tissue samples were incubated with QZ probe MC225-Sub2 for 48 hours at 37 C. Data indicate that protease activity was preserved in tissue section even after two freeze-thaw cycles (Table 4).

TABLE 4
Protease activity (%) vs. Number of freeze-thaw cycles

| | | 1 freeze- | 2 freeze- |
|---|---|---|---|
| Sample ID | No thaw | thaw cycle | thaw cycles |
| 131068 | 42% | 43% | 31% |
| | 30% | 32% | 45% |
| H292 7.1 | 73% | 56% | 68% |
| | 66% | 71% | 50% |

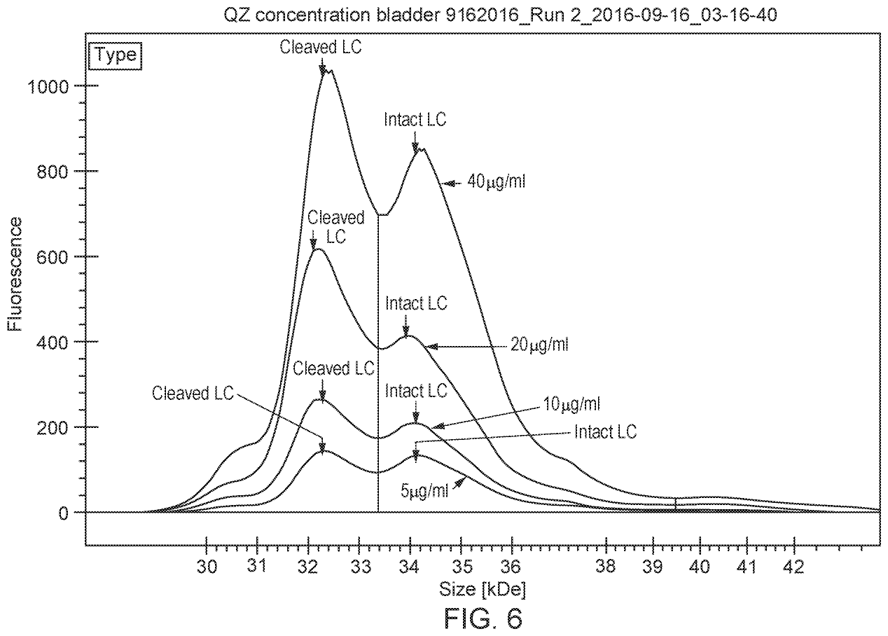

QZ probe Concentration. Protease activity of tissue samples was tested using a serial dilution of QZ probe concentrations (5 μg/mL, 10 μg/mL, 20 μg/mL and 40 μg/mL). No significant difference in protease activity was observed after 24-hour incubation on the tissue sample with QZ probe MC225-Sub1 in the concentration range of from 5 μg/mL to 40 μg/mL (FIG. 6 and Table 5).

TABLE 5
Protease activity (%) in tissue sample
#40846 vs. QZ probe concentration

| QZ probe concentration | 40 | 20 | 10 | 5 |
|---|---|---|---|---|
| (μg/mL) | μg/mL | μg/mL | μg/mL | μg/mL |
| Protease activity (%) | 49.40% | 56.05% | 50.61% | 46.04% |

Tissue Sampling. Protease activity was assessed in tissue samples taken from the center area of the tissue section and from over 100 μm away from the center area. No significant difference in protease activity was observed after 24-hour incubation on the tissue sample with QZ probe MC225-Sub1 in these two locations, indicating that areas with similar cellular content in the same tissue section will likely result in similar protease activity (Table 6).

TABLE 6
Protease activity in different locations of a tissue section

| Distance from Center | Protease |
|---|---|
| Area of Tissue Section | activity (%) |
| 1 μm | 57.52% |
| 132 μm | 58.30% |

### Example 3

### Assessment of Protease Activity in H292 Xenograft Tumor Sections

A series of QZ probes containing either a serine protease substrate, a matrix metalloproteinase (MMP)-cleavable substrate, or a substrate cleavable by both serine protease and MMP, were used to assess protease activity in H292 xenograft tumor sections. Each probe was an activatable antibody or an activatable pseudo-antibody that employed the light chain as the bipartite polypeptide having the structure of, from N-terminus to C terminus, B-CL-A. Table 7 indicates the substrate sequence within the CL of each QZ probe.

TABLE 7
QZ probes

| QZ Probe | Substrate | Target Protease |
|---|---|---|
| C225-S01 | LSGRSDNH | Serine protease |
| | (SEQ ID NO: 17) | |
| C225-M01 | PLGL | MMP |
| | (SEQ ID NO: 84) | |
| MC225-Sub 1 | ISSGLLSGRSDNH | Serine protease and MMP |
| | (SEQ ID NO: 40) | |
| MC225-Sub2 | AVGLLAPPGGLSGRSDNH | Serine protease and MMP |
| | (SEQ ID NO: 41) | |
| Probe A | ISSGLLSS | MMP |
| | (SEQ ID NO: 31) | |
| Probe B | LSGRSDNH | Serine protease |
| | (SEQ ID NO: 17) | |
| Probe C | ISSGLLSGRSDNH | Serine protease and MMP |
| | (SEQ ID NO: 40) | |
| Probe D | AVGLLAPP | MMP |
| | (SEQ ID NO: 26) | |
| Probe E | LSGRSDNH | Serine protease |
| | (SEQ ID NO: 17) | |
| Probe F | AVGLLAPPGGLSGRSDNH | Serine protease and MMP |
| | (SEQ ID NO: 41) | |

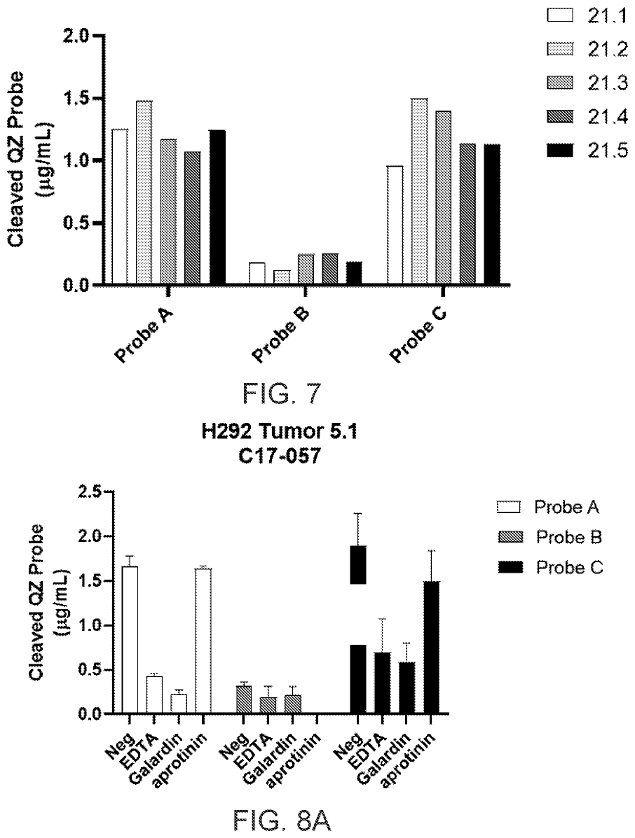

To evaluate the heterogeneity of protease activity in tumor tissues, cleavage of QZ Probes A-F were measured in H292 cell line derived xenograft tumor sections. Probe A and Probe D each carries an MMP-specific substrate, while Probe B and Probe E each carries a serine protease-specific substrate (note Probe B and Probe E are identical), Probe C and Probe F E each carries a substrate which can be cleaved by both MMP and serine protease. Using five independent H292 xenografts, QZ assays showed higher protease activities in the tumor sections incubated with either Probe A or Probe C. Probe A and Probe C both contains an MMP-cleavable substrate (Probe A substrate: ISSGLLSS (SEQ ID NO:31), Probe C substrate: ISSGLLSGRSDNH (SEQ ID NO:40)), indicating that cleavage activities in the H292 xenograft tumor samples are largely driven by MMP proteases, not serine proteases (FIG. 7).

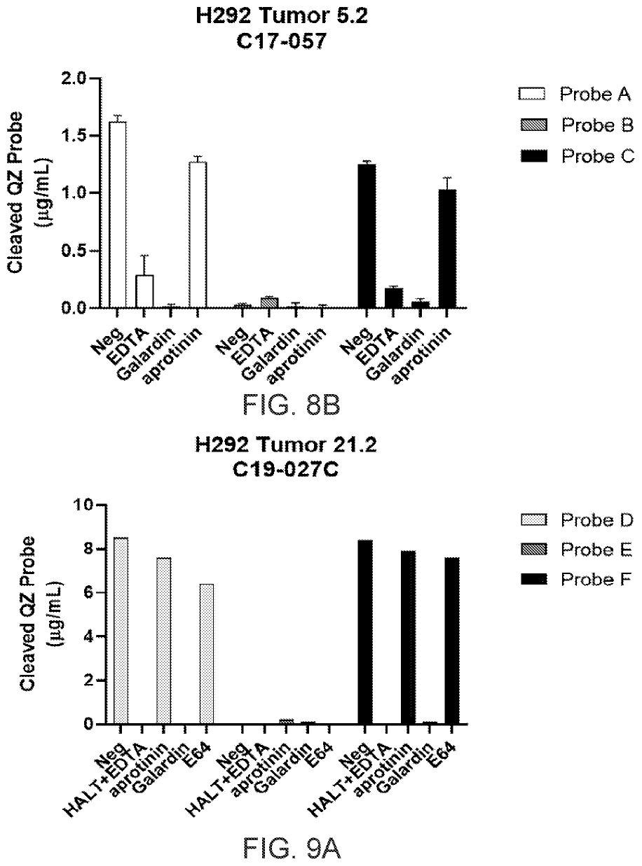

To further confirm that the QZ assay can distinguish different protease specificities, protease activity in H292 xenograft tumor tissues was assessed with different protease inhibitors. 20 μg/mL QZ Probes A-C were incubated for 48 h at 37 C in H292 xenograft tumor samples 5.1 and 5.2 (n=2), in the absence of protease inhibitors (Neg) or in tumor samples pre-incubated with EDTA (broad metalloprotease inhibitor), Galardin (MMP inhibitor), and aprotinin (serine protease inhibitor) (FIGS. 8A and 8B). The results showed a significant decrease of protease activities detected by Probe A and Probe C in the tumor sections when pre-incubated with Galardin, a MMP inhibitor, further confirming a predominant MMP activity in H292 derived xenograft tumor sections.

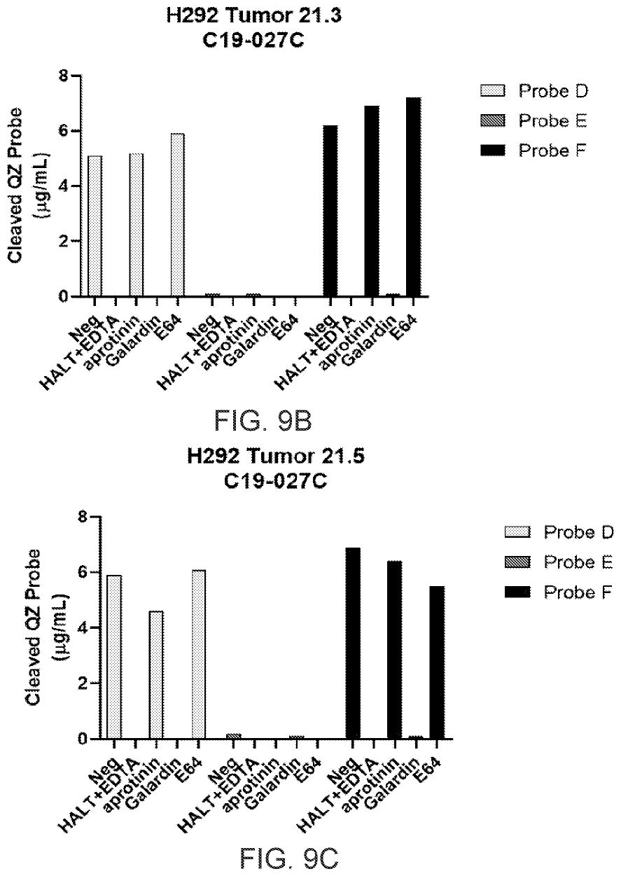

Similarly, 20 μg/mL QZ Probes D-E were incubated for 48 h at 37 C in H292 xenograft tumor samples 21.2, 21.3, and 21.5 (n=3), in the absence of protease inhibitors (Neg) or in tumor samples pre-incubated with a broad-spectrum protease inhibitor cocktail (HALT+EDTA), a MMP protease inhibitor (Galardin), a cysteine protease inhibitor (E64), and a serine protease inhibitor (aprotinin) (FIGS. 9A-C). Consistent with the results from previous assays using Probes A-C, QZ assay assessments with protease inhibitors using Probes D, B, and E showed higher protease activities in the tumor sections incubated with Probe D or Probe E which carries an MMP-specific substrate (AVGLLAPP, SEQ ID NO:26). These protease activities were specifically blocked by Galardin, a MMP inhibitor, further demonstrating a predominant MMP activity in H292 derived xenograft tumor sections.

### Example 4

### Assessment of Protease Activity in Patient-Derived Tumor Samples

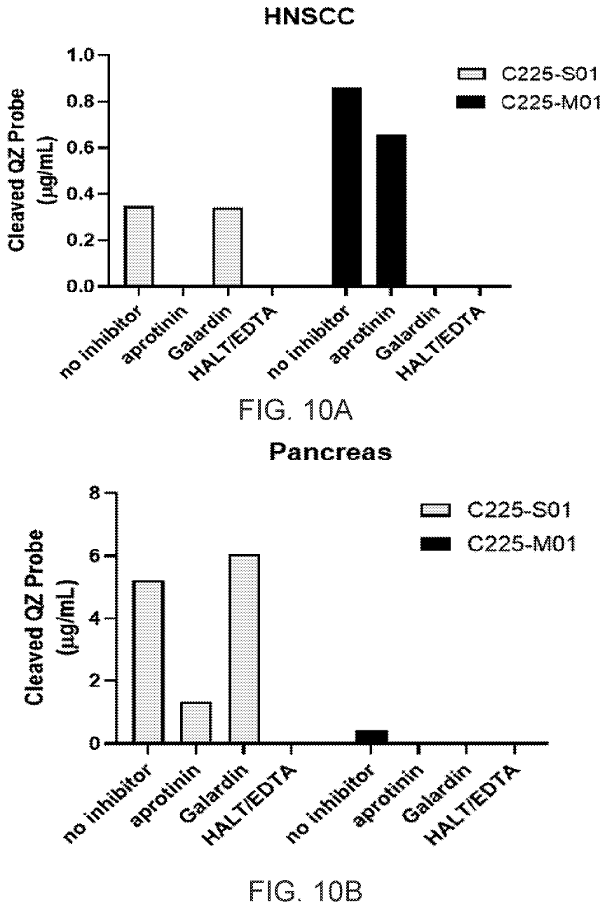

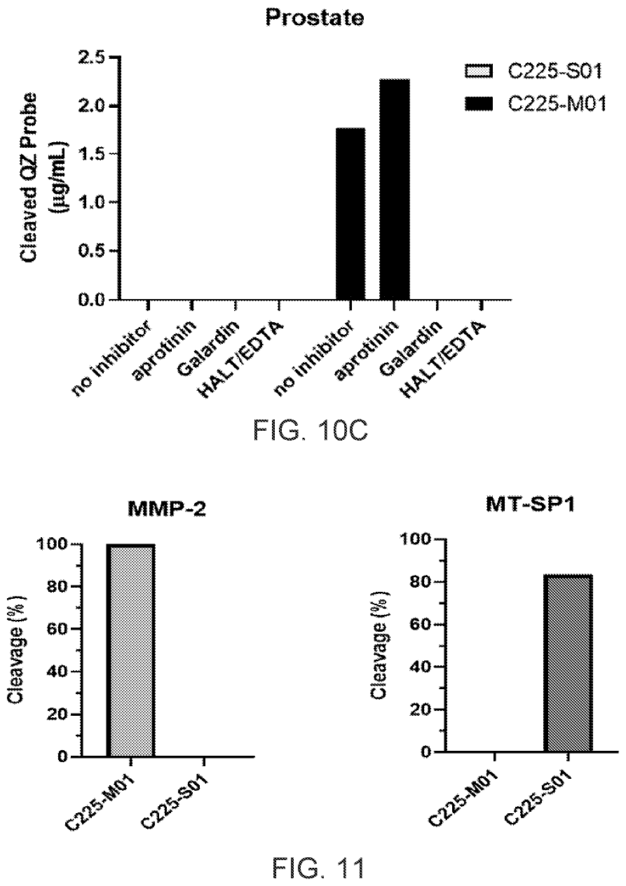

To further evaluate the heterogeneity of protease activity in patient tumor tissues, QZ probes C225-S01 and C225-M01 (activatable antibody probes that employ light chains as bipartite polypeptides having the structure of, from N-terminus to C-terminus, B-CL-A, where B is a parental masking moiety) were used to assess protease activity in tumor samples from patients with different types of cancer: head and neck squamous cell carcinoma (HNSCC) (FIG. 10A), pancreatic cancer (FIG. 10B), and prostate cancer (FIG. 10C). QZ probe C225-S01 contains a serine protease substrate (LSGRSDNH (S01), SEQ ID NO:17) cleavable by at least two serine proteases, MT-SP1 and urokinase-type plasminogen activator, and QZ probe C225-M01 which contains a broad-spectrum matrix metalloproteinase (MMP)-specific substrate (PLGL (M01), SEQ ID NO:84).

Protease inhibitors were used to confirm the specificity of the protease activity measured by both QZ probes. Notably, the HNSCC tumor sample revealed the presence of both serine and MMP protease activities. As expected, the cleavage signal of both C225-S01 and C225-M01 QZ probes was inhibited by pre-treatment of the tissues with a broad-spectrum protease inhibitor cocktail. However, whereas C225-S01 cleavage was abolished by the serine protease-specific inhibitor aprotinin and not the MMP-specific inhibitor Galardin, a reverse inhibition pattern was detected for C225-M01 (FIG. 10A). These data thus demonstrate that the QZ assay can differentiate between different protease specificities by exploiting substrate specificity and selectivity and that further refinement can be achieved using protease-specific inhibitors. Additional tissue samples of human pancreatic and prostate tumors were identified as examples of tissues with predominant serine protease or MMP activity, respectively, using the QZ probe and protease class-specific inhibitors (FIGS. 10B, 10C).

To further demonstrate protease selectivity of the QZ probes, C225-M01 and C225-S01 were cross tested against recombinant human MMP-2 (4 h) and MT-SP1 (24 h), and percent cleavage was measured by capillary electrophoresis (CE) (FIG. 11). QZ probe cleavage assessment showed no cross reactivity for the purified proteases, demonstrating the MMP and serine protease class selectivity of C225-M01 and C225-S01, respectively.

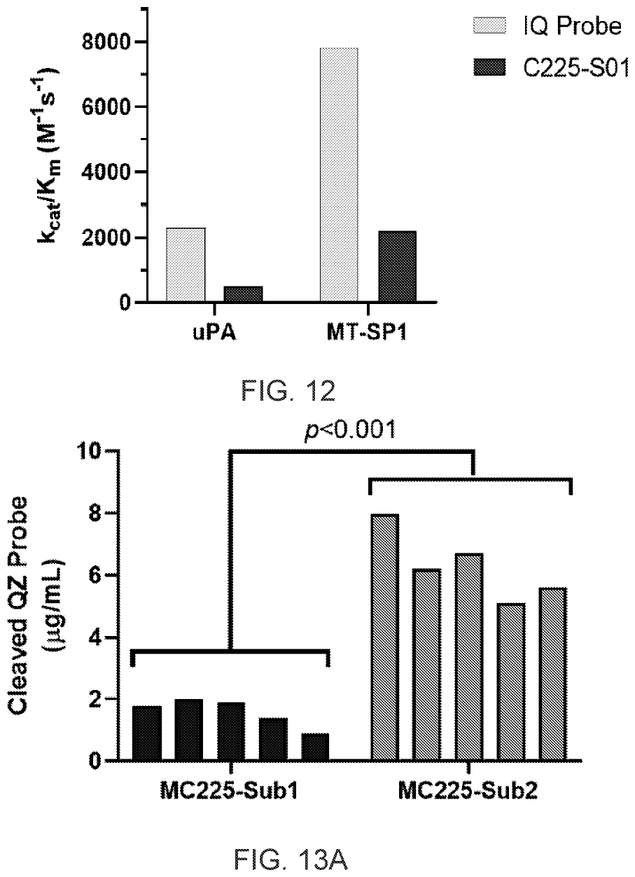

Because cleavability of a given protease substrate can be affected by the scaffold in which it is presented, two QZ probes containing the same S01 substrate in different molecular structures/sizes were evaluated. IQ Probe was represented as a small QZ probe, in the format of an internally quenched (IQ) linear polypeptide containing a S01 substrate (SEQ ID NO:17). C225-S01 was representative of a large QZ probe, in the format of an activatable antibody which comprises the two heavy chains of C225, and two bipartite polypeptides (i.e., light chains), each having the structure of, from N-terminus to C-terminus, B-CL-A, wherein each component A comprises a light chain variable domain and a light chain constant domain of C225, and each component B is the masking moiety, and CL comprises the S01 substrate. The k_{cat}/K_{M} of the S01 substrate in both small and large QZ probes were determined in the presence of a serine protease. The k_{cat}/K_{M} of the S01 substrate was higher in the context of the small probe (IQ Probe) compared to the large probe (C225-S01) for two tested proteases uPA and MT-SP1: 4.6× and 3.5× times, respectively (FIG. 12), indicating a substrate is more accessible to target protease/s in the format of small probes (e.g., IQ probes) or other small molecule formats, compared to more complex, larger molecules, such as antibodies. Thus, assessment of substrate activation in biological tissues using surrogate small molecules and fluorescent probes can be misleading by potentially overestimating the degree of substrate cleavage, and thus should be avoided for the development of conditionally activated therapeutics based on large molecules, such as antibodies.

### Example 5

### Characterization of Protease Activity in H292 Xenograft Tumor Sections

To assess whether protease activity measured in situ with the QZ assay correlates with in vivo activatable antibody efficacy, the EGFR-responsive H292 xenograft model was used. Eight-week-old female Fox Chase severe combined immunodeficiency mice (Charles River Laboratories) were implanted subcutaneously in the right hind flank with 5×10⁶ H292 cells. The implant medium contained a 1:1 mixture of serum-free RPMI media and Matrigel (Corning). After tumors were palpable, body weights and tumor volumes were collected twice weekly. Tumors were measured using digital calipers and tumor volumes calculated using the formula: tumor length×(tumor width)²×0.52=tumor volume. Eleven days after tumor cell implantation, mice were assigned to treatment groups of eight mice each. Each treatment group had approximately equal mean tumor volumes of 140 mm³. After assignment, mice were treated with a single 10 mg/kg intraperitoneal dose of test article.

In these experiments, two activatable antibodies, C225-Sub1 and C225-Sub2, containing the multi-specific protease substrates Sub1 (SEQ ID NO: 40) and Sub2 (SEQ ID NO: 41), respectively, were utilized in the in vivo efficacy study, while two activatable pseudo-antibodies, MC225-Sub1 and MC225-Sub2, were utilized as QZ probes in the in situ QZ assay. The light chains in each of the QZ probes function as a bipartite polypeptide in which component A comprises each light chain antibody or pseudo-light chain antibody component and each component B is a parental masking moiety. The light chains of C225-Sub1 and MC225-Sub1 are identical (SEQ ID NO: 7). The light chains of C225-Sub2 and MC225-Sub2 are also identical (SEQ ID NO: 8).

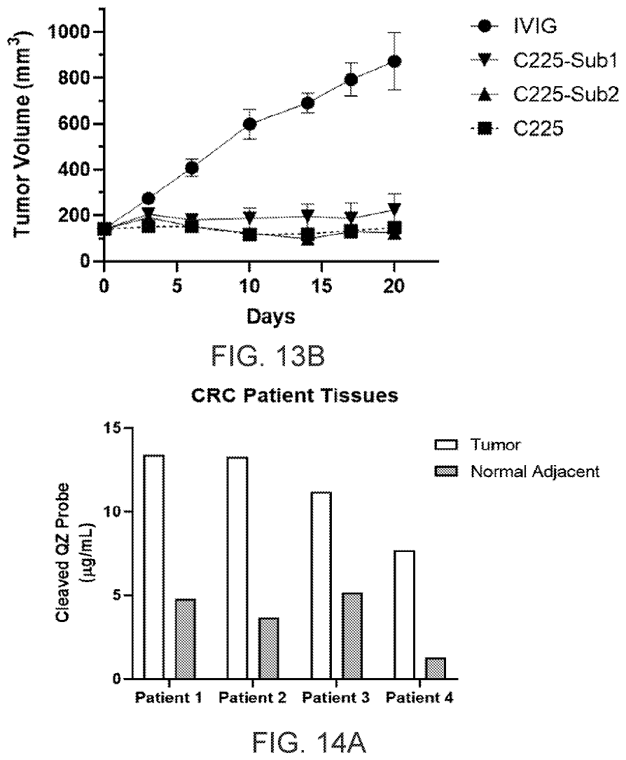

Based on the substrate designs, substrate Sub1 is less cleavable by selected MMP proteases than Sub2 and therefore the QZ probe MC225-Sub1 demonstrated a lower cleavage rate in situ compared to MC225-Sub2 (FIG. 13A). This finding was consistent across several H292 xenograft tumors from different mice (n=5). Notably, this in situ cleavage profile was corroborated by the respective in vivo efficacies of the activatable antibodies in the H292 xenograft tumor model (FIG. 13B). While both the activatable antibodies C225-Sub1 and C225-Sub2 demonstrated significant efficacy at the 10-mg/kg dose, efficacy was enhanced with C225-Sub2 compared with C225-Sub1. These data demonstrate translation of the in situ QZ assay to the biological activity of activatable antibodies within in vivo models and highlight a potential role for the QZ assay in prediction of protease substrate cleavage in biological systems.

### Example 6

### Evaluation of Protease Activity in Patient-Derived Tumor Samples

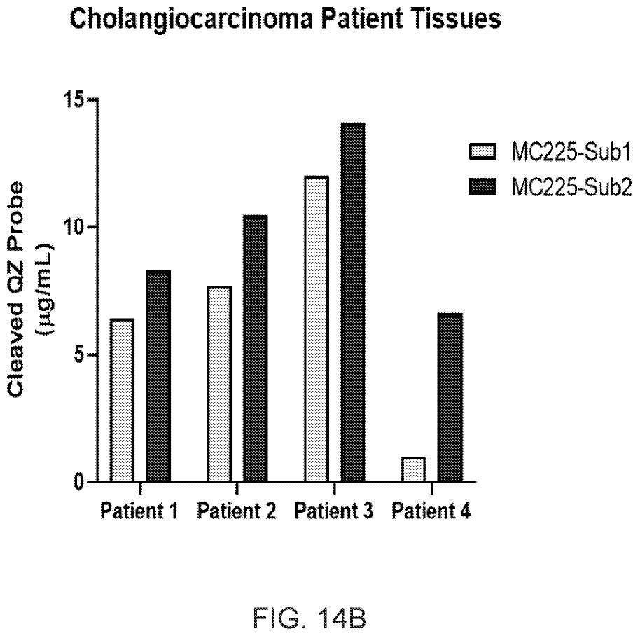

Protease activities in patient-derived tumor samples were evaluated using the QZ assay. Tumor tissue samples and adjacent normal colon tissue samples from four colorectal cancer (CRC) patients were analyzed utilizing QZ probe MC225-Sub2. As expected, higher protease activities were observed in tumor sections compared to normal adjacent sections in all four patients (FIG. 14A). Furthermore, tumor tissue samples from four cholangiocarcinoma patients were examined using QZ probes MC225-Sub1 and MC225-Sub2. Consistent with the H292 xenograft tumor results, a slightly higher cleavage rate was observed with MC225-Sub2 compared with MC255_Sub1 in all four patient tumor samples (FIG. 14B). These results further demonstrate that the QZ assay enables the ability to assess the level of protease activity in patient tissues and supports the design of specific protease substrates that leverage the activities of tumor-associated proteases for therapeutic activation.

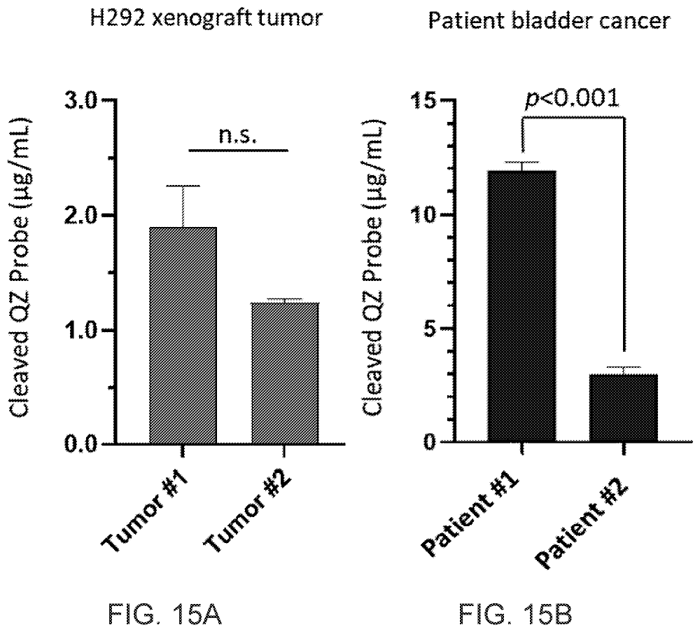

To evaluate the reproducibility of the QZ assay, QZ probe MC225-Sub1 was used to examine protease activities in serial sections of H292 derived xenograft tumor samples (n=2) (FIG. 15A) and patient bladder cancer tumor sections (n=4) from two patients (FIG. 15B). These data support reproducibility of the QZ assay between different tissue sections of the same tumor sample and independent rounds of the assay. As expected, more variability is detected between different patients' tumors (FIG. 15B); however, the data from two tumors of the same mouse xenograft model demonstrate similar protease activity (FIG. 15A). Data are presented as mean standard error. Statistical significance was analyzed using the two-sided Student's t-test (p<0.05 was considered statistically significant).

### Example 7

### Assessment of Protease Activity in Patient-Derived Plasma Samples

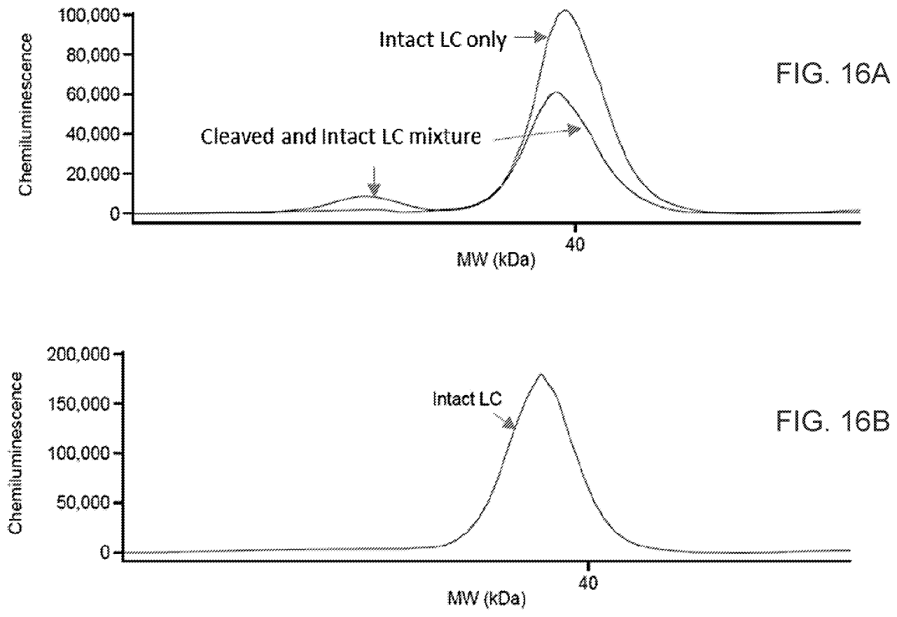

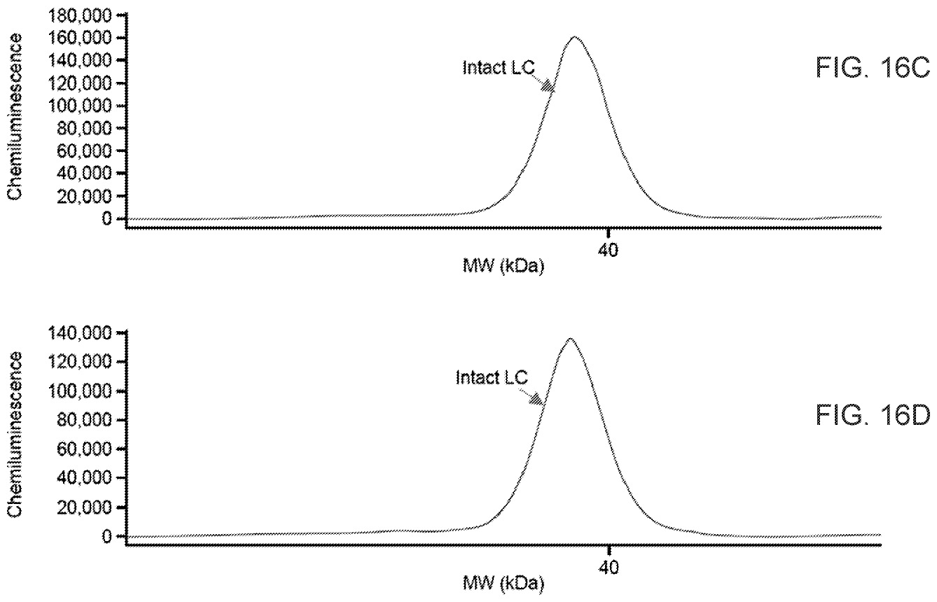

The QZ assay of the present invention can be used to detect protease activity in liquid as well as in solid biological samples. Here, an example is presented of the assessment of a QZ probe comprising an activatable anti-PD-1 antibody therapeutic (HC, SEQ ID NO: 15; LC, SEQ ID NO: 16) was used to assess protease activity in the plasma of heathy donors and patients with cancers. The activatable anti-PD-1 antibody therapeutic has two heavy chains and two light chains, in which each light chain functions as a bipartite polypeptide where component A comprises a light chain variable domain and a light chain constant domain and component B comprises a masking moiety in the structure of, from N-terminus to C-terminus, B-CL-A. It was first established that a capillary electrophoresis immunoassay-based method effectively differentiated the cleaved light chain (LC) and intact LC (intact bipartite polypeptide) by running a mix of cleaved LC and intact LC at a 2:8 ratio (FIG. 16A). The lower limit of detection of cleaved LC was determined to be ≥1 μg/mL. After 48 hours of incubation of the QZ probe in plasma samples from normal healthy volunteers (FIG. 16B, Table 8, n=5) or patients with lung cancer (FIG. 16C, Table 9, n=5) or patients with gastric cancer (FIG. 16D, Table 10, n=5), there was no evidence of light chain cleavage, thereby suggesting that the activatable anti-PD1 antibody therapeutic is stable in patients' plasma as designed.

TABLE 8
Test Sample: Plasma from Healthy Human Donors

| Sample | Clinical | | | Probe |
|---|---|---|---|---|
| No. | Diagnosis | Location | Stage | Cleavage |
| 01 | Normal | N/A | N/A | No cleavage |
| | | | | detected |
| 02 | Normal | N/A | N/A | No cleavage |
| | | | | detected |
| 03 | Normal | N/A | N/A | No cleavage |
| | | | | detected |
| 04 | Normal | N/A | N/A | No cleavage |
| | | | | detected |
| 05 | Normal | N/A | N/A | No cleavage |
| | | | | detected |

N/A = not applicable.

TABLE 9
Test Sample: Plasma from Lung Cancer Patients

| Sample | Clinical | | | Probe |
|---|---|---|---|---|
| No. | Diagnosis | Location | Stage | Cleavage |
| 06 | Lung cancer | Upper lobe of | IIB | No cleavage |
| | | left lung | | detected |
| 07 | Lung cancer | Upper lobe of | IB | No cleavage |
| | | right lung | | detected |
| 08 | Lung cancer | Lower lobe of | IB | No cleavage |
| | | left lung | | detected |
| 09 | Lung cancer | Upper lobe of | IIB | No cleavage |
| | | right lung | | detected |
| 10 | Lung cancer | Lower lobe of | IB | No cleavage |
| | | right lung | | detected |

TABLE 10
Test Sample: Plasma from Gastric Cancer Patients

| Sample | Clinical | Histological | | | Probe |
|---|---|---|---|---|---|
| No. | Diagnosis | Diagnosis | Grade | Stage | Cleavage |
| 11 | Stomach | Adenocarcinoma | G3 | IIA | No cleavage |
| | cancer | | | | detected |
| 12 | Stomach | Adenocarcinoma | G2 | IB | No cleavage |
| | cancer | | | | detected |
| 13 | Stomach | Adenocarcinoma | G2-3 | IIIB | No cleavage |
| | cancer | and signet ring | | | detected |
| | | cell carcinoma | | | |
| 14 | Stomach | Adenocarcinoma | G2 | IIA | No cleavage |
| | cancer | | | | detected |
| 15 | Stomach | Adenocarcinoma | G2-3 | IIA | No cleavage |
| | cancer | | | | detected |

### Example 8

Assessment of Protease Activity in Biological Samples with Mass Spectrometry (Prophetic)

The QZ assay of the present invention can be used to detect protease activity in biological matrices, such as tissues, cells, or liquid samples, using mass spectrometry (MS). In this example, MS-based detection will enable quantification of QZ probe cleavage and identification of protease cleavage site(s) within the QZ probe polypeptide sequence. The biological sample will be incubated with the QZ probe in MS-compatible assay buffer (AB). In the case of QZ assays using tissue sections, a hydrophobic barrier will be drawn around each tissue sample to maintain liquid on the tissue. The QZ probe will be incubated with each biological sample or in AB only; for example, this may be performed in a humidified chamber at 37° C. for up to 48 hours. Following incubation, the QZ probe may be isolated from the biological sample using an affinity capture reagent, such as a capture antibody or affinity resin. The QZ probe may be chemically modified or produced with an affinity tag to enable capture from a biological matrix. The QZ probe may be further processed to remove glycosylation and/or reduced to remove disulfide crosslinks. The QZ probe may be resolved by liquid chromatography or capillary electrophoresis prior to MS detection. MS detection may be performed using electrospray ionization mass spectrometry (ESI MS). Comparison of the observed mass(es) of the QZ probe to the expected mass(es) will enable the site(s) of cleavage in the polypeptide to be determined. The fraction of the QZ probe in the biological samples or AB controls with cleavage will be determined by quantifying the fraction of cleaved polypeptide determined from the deconvoluted MS spectra.

### Example 9

Assessment of Protease Activity in Tissues with a Protease-Activatable Cytokine

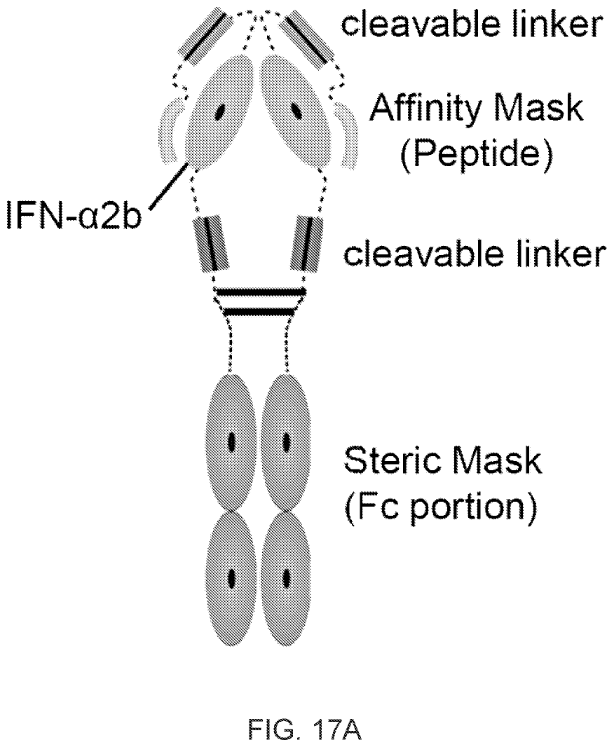

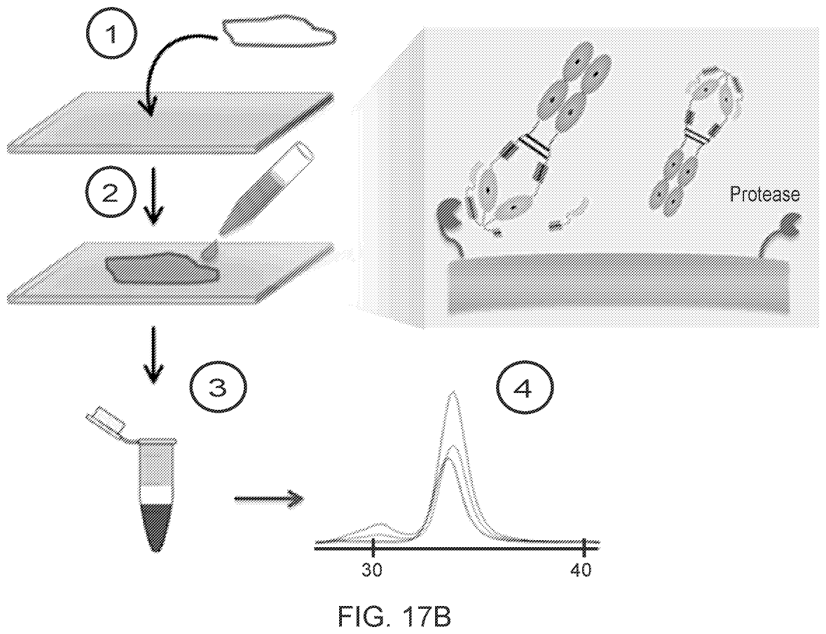

The QZ assay of the present invention can be used to detect protease activity in tissue samples using a protease-activatable cytokine. Here, an example is presented of the assessment of a QZ probe comprising an activatable interferon (IFN)-α2b therapeutic (SEQ ID NO: 85). The activatable IFN-α2b therapeutic has a peptide affinity masking moiety fused to the N-terminus of human IFN-α2b via a protease-cleavable linker and a constant fragment (Fc) steric masking moiety fused to the C-terminus of human IFN-α2b through a second protease-cleavable linker (FIG. 17A). Incubation of a fluorescently labeled IFN-α2b QZ probe with a tissue section enables the measurement of protease activity in the tissue through protease cleavage of the QZ probe (FIG. 17B). In this example, protease cleavage of the IFN-α2b QZ probe with tissues is assessed through capillary electrophoresis. Changes in IFN-α2b QZ probe functional activity during tissue incubation are also assessed with a cell-based reporter assay.

### Example 10

### Assessment of Protease Activity in Patient-Derived Tissue Samples

Using the IFN-α2b QZ probe described in Example 9, the protease activity in tumor sections of 12 μm thickness was assessed from human triple negative breast cancer (TNBC), head and neck (H&N) cancer, and non-small cell lung cancer (NSCLC) patient tissues. In addition, the protease activity was assessed by applying the IFN-α2b QZ probe to normal tissue sections of 12 μm thickness from Cynomolgus monkey brain (cortex), breast, liver, and skin. A hydrophobic barrier was drawn around each tissue sample to maintain liquid on the tissue using an ImmEdge™ Hydrophobic Barrier Pen (Vector Laboratories). The tissues were then incubated with 20 μg/mL AF647-labeled IFN-α2b QZ probe in buffer consisting of 150 mM Tris HCl pH 7.4, 5 mM CaCl₂) 100 μM ZnCl2, and 0.005% Tween®-20 (QZ assay buffer). The QZ probe was incubated with each tissue section or in QZ buffer alone in a humidified chamber at 37° C. for up to 48 hours. A separate tissue section was used for each QZ assay time point.

Following QZ probe incubation, tissue supernatants and no-tissue controls were collected and transferred into a well of a 96-well PCR plate for assay by capillary electrophoresis. Each supernatant sample was mixed with Pico Sample Buffer (Perkin Elmer) containing 2-beta-mercaptoethanol at four parts sample and one part of Pico Sample Buffer and then heated at 95° C. for 10 minutes. The composition of each supernatant sample was then assessed using the LabChip GXII Touch (Perkin Elmer) with the HT Pico Protein Express 100 protocol (Perkin Elmer). Protein Express Assay LabChips (Perkin Elmer #760499) were set up using the protocol of the Protein Pico Assay Reagent Kit (Perkin Elmer #760498). The fraction of the QZ probe in the tissue supernatants or no-tissue controls with IFN-Fc cleavage was determined by quantifying the fraction of the polypeptide corresponding to the released Fc using the equation Fc=(Fc+intact IFN). Quantitation was performed using the LabChip GX Reviewer software (Perkin Elmer). QZ probe functional activity in non-denatured tissue supernatants or no-tissue controls was tested in a separate assay using a HEK-Blue™ IFN-α/β reporter cell line kit (InvivoGen, hkb-ifnab) that measures type 1 interferon pathway activation through quantification of secreted embryonic alkaline phosphatase (SEAP) levels with QUANTI-Blue™ solution.

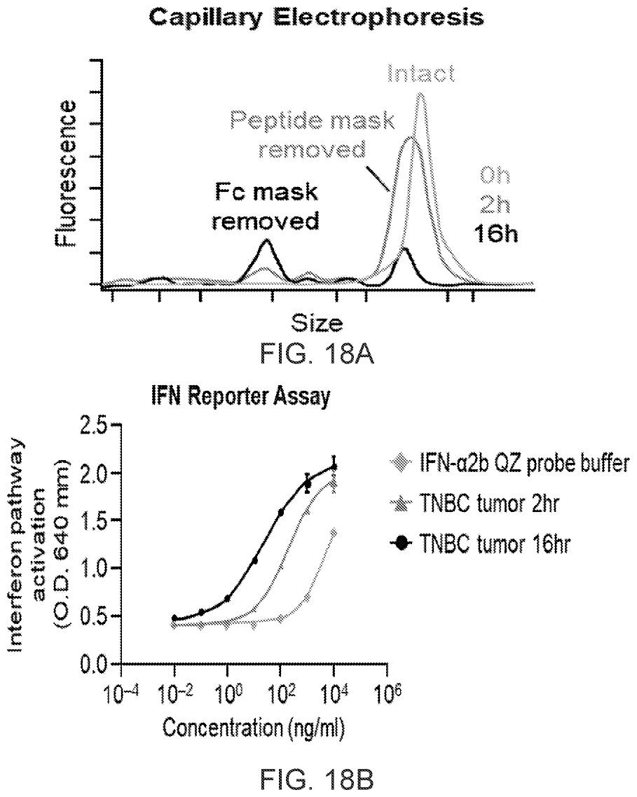

IFN-α2b QZ probe incubated with tumor tissue sections from TNBC, H&N, and NSCLC patients (Table 11A) demonstrated IFN-α2b QZ probe cleavage as measured by capillary electrophoresis. Notably, no measurable QZ probe cleavage was detected in the Cynomolgus monkey normal tissue sections (Table 11B) after 48 h of incubation. A separate assay time course was performed with the TNBC tumor tissue sections to correlate IFN-α2b QZ probe cleavage by capillary electrophoresis (FIG. 18A) and the IFN-α2b functional reporter assay using the HEK-Blue™ reporter cell line (FIG. 18B). These results show that time-dependent IFN-α2b QZ probe cleavage on tumor tissues is correlated with an increase in unmasked IFN-α2b functional activity. Together, these results demonstrate that capillary electrophoresis and/or cytokine functional activity readouts can be used to measure protease-activatable cytokine cleavage in biological samples.

TABLE 11A
IFNα2b QZ probe cleavage with human
tumor tissue sections after 48 h incubation

| | TNBC Tissue | H&N Tissue | NSCLC Tissue |
|---|---|---|---|
| | Cleaved | Cleaved | Cleaved |
| | IFN-Fc | IFN-Fc | IFN-Fc |
| QZ Probe | (μg/mL) | (μg/mL) | (μg/mL) |
| IFNa2b AF647 | 20.0 | 7.6 | 15.2 |

TABLE 11B
IFNα2b QZ probe cleavage with Cynomolgus monkey
normal tissue sections after 48 h incubation

| | Brain (cortex) | Breast | Liver | Skin |
|---|---|---|---|---|
| | Cleaved | Cleaved | Cleaved | Cleaved |
| | IFN-Fc | IFN-Fc | IFN-Fc | IFN-Fc |
| QZ Probe | (μg/mL) | (μg/mL) | (μg/mL) | (μg/mL) |
| IFNa2b AF647 | 0.0 | 0.0 | 0.0 | 0.0 |

Table 12 provides a listing of amino acid sequences referred to herein.

TABLE 12
Table of Sequences

| SEQ | | |
|---|---|---|
| ID NO | Name | Sequence |
| 1 | C225_HC | QVQLKQSGPGLVQPSQSLSITCTVSGFSLTNYGVHWVRQSP |
| | | GKGLEWLGVIWSGGNTDYNTPFTSRLSINKDNSKSQVFFKM |
| | | NSLQSQDTAIYYCARALTYYDYEFAYWGQGTLVTVSAAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 2 | C225_LC | QILLTQSPVILSVSPGERVSFSCRASQSIGTNIHWYQQRINGS |
| | | PRLLIKYASESISGIPSRFSGSGSGTDFTLSINSVESEDIADYYC |
| | | QQNNNWPTTFGAGTKLELKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFN |
| | | RGEC |
| 3 | MC225_HC | QVQLKQSGPGLVQPSQSLSITCTVSGFSLTSYGVHWVRQSP |
| | | GKGLEWLGVIWSKGSTAYNTPFTSRLSINKDNSKSQVFFKM |
| | | NSLQSQDTAIYYCARALTSGKAEFAYWGQGTLVTVSAAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 4 | MC225_LC | QILLTQSPVILSVSPGERVSFSCRASQSIGTNIHWYQQRINGS |
| | | PRLLIKYASESISGIPSRFSGSGSGTDFTLSINSVESEDIADYYC |
| | | QQNNNWPTTFGAGTKLELKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFN |
| | | RGEC |
| 5 | C225-S01_LC | QGQSGQCISPRGCPDGPYVMYGSSGGSGGSGGSGLSGRSD |
| | | NHGSSGTQILLTQSPVILSVSPGERVSFSCRASQSIGTNIHWY |
| | | QQRTNGSPRLLIKYASESISGIPSRFSGSGSGTDFTLSINSVES |
| | | EDIADYYCQQNNNWPTTFGAGTKLELKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| 6 | C225-M01_LC | QGQSGQCISPRGCPDGPYVMYGSSGGSGGSGGSGPLGLGSS |
| | | GTQILLTQSPVILSVSPGERVSFSCRASQSIGTNIHWYQQRTN |
| | | GSPRLLIKYASESISGIPSRFSGSGSGTDFTLSINSVESEDIADY |
| | | YCQQNNNWPTTFGAGTKLELKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC |
| 7 | MC225- | QGQSGQCISPRGCPDGPYVMYGSSGGSGGSGGSGISSGLLS |
| | Sub1_LC | GRSDNHGSSGTQILLTQSPVILSVSPGERVSFSCRASQSIGTNI |
| | | HWYQQRTNGSPRLLIKYASESISGIPSRFSGSGSGTDFTLSINS |
| | | VESEDIADYYCQQNNNWPTTFGAGTKLELKRTVAAPSVFIF |
| | | PPSDEQLKSGTASVVCLLNNFYPREAKVQWKVDNALQSGN |
| | | SQESVTEQDSKDSTYSLSSTLTLSKADYEKHKVYACEVTHQ |
| | | GLSSPVTKSFNRGEC |
| 8 | MC225- | QGQSGQCISPRGCPDGPYVMYGSSGGSGGSGGSGAVGLLA |
| | Sub2_LC | PPGGLSGRSDNHGSSGTQILLTQSPVILSVSPGERVSFSCRAS |
| | | QSIGTNIHWYQQRTNGSPRLLIKYASESISGIPSRFSGSGSGT |
| | | DFTLSINSVESEDIADYYCQQNNNWPTTFGAGTKLELKRTV |
| | | AAPSVFIFPPSDEQLKSGTASVVCLLNNFYPREAKVQWKVD |
| | | NALQSGNSQESVTEQDSKDSTYSLSSTLTLSKADYEKHKVY |
| | | ACEVTHQGLSSPVTKSFNRGEC |
| 9 | Probe A_LC | QGQSGQCISPRGCPDGPYVMYGSSGGSGGSGGSGISSGLLSS |
| | | GSSGTQILLTQSPVILSVSPGERVSFSCRASQSIGTNIHWYQQ |
| | | RTNGSPRLLIKYASESISGIPSRFSGSGSGTDFTLSINSVESEDI |
| | | ADYYCQQNNNWPTTFGAGTKLELKRTVAAPSVFIFPPSDEQ |
| | | LKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 10 | Probe B_LC | QGQSGQCISPRGCPDGPYVMYGSSGGSGGSGGSGLSGRSDN |
| | | HGSSGTQILLTQSPVILSVSPGERVSFSCRASQSIGTNIHWYQ |
| | | QRTNGSPRLLIKYASESISGIPSRFSGSGSGTDFTLSINSVESE |
| | | DIADYYCQQNNNWPTTFGAGTKLELKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 11 | Probe C_LC | QGQSGQCISPRGCPDGPYVMYGSSGGSGGSGGSGISSGLLSG |
| | | RSDNHGSSGTQILLTQSPVILSVSPGERVSFSCRASQSIGTNIH |
| | | WYQQRTNGSPRLLIKYASESISGIPSRFSGSGSGTDFTLSINSV |
| | | ESEDIADYYCQQNNNWPTTFGAGTKLELKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQGL |
| | | SSPVTKSFNRGEC |
| 12 | Probe D_LC | QGQSGQCISPRGCPDGPYVMYGSSGGSGGSGGSGAVGLLAP |
| | | PGSSGTQILLTQSPVILSVSPGERVSFSCRASQSIGTNIHWYQ |
| | | QRTNGSPRLLIKYASESISGIPSRFSGSGSGTDFTLSINSVESE |
| | | DIADYYCQQNNNWPTTFGAGTKLELKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 13 | Probe E_LC | QGQSGQCISPRGCPDGPYVMYGSSGGSGGSGGSGLSGRSDN |
| | | HGSSGTQILLTQSPVILSVSPGERVSFSCRASQSIGTNIHWYQ |
| | | QRTNGSPRLLIKYASESISGIPSRFSGSGSGTDFTLSINSVESE |
| | | DIADYYCQQNNNWPTTFGAGTKLELKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 14 | Probe F_LC | QGQSGQCISPRGCPDGPYVMYGSSGGSGGSGGSGAVGLLAP |
| | | PGGLSGRSDNHGSSGTQILLTQSPVILSVSPGERVSFSCRASQ |
| | | SIGTNIHWYQQRINGSPRLLIKYASESISGIPSRFSGSGSGTDF |
| | | TLSINSVESEDIADYYCQQNNNWPTTFGAGTKLELKRTVAA |
| | | PSVFIFPPSDEQLKSGTASVVCLLNNFYPREAKVQWKVDNA |
| | | LQSGNSQESVTEQDSKDSTYSLSSTLTLSKADYEKHKVYAC |
| | | EVTHQGLSSPVTKSFNRGEC |
| 15 | HC | EVQLVESGGGLVQPGGSLRLSCAASGFTFSGYAMSWVRQA |
| | | PGKGLEWVAYISNSGGNAHYADSVKGRFTISRDNSKNTLYL |
| | | QMNSLRAEDTAVYYCTREDYGTSPFVYWGQGTLVTVSSAS |
| | | TKGPSVFPLAPCSRSTSESTAALGCLVKDYFPEPVTVSWNSG |
| | | ALTSGVHTFPAVLQSSGLYSLSSVVTVPSSSLGTKTYTCNVD |
| | | HKPSNTKVDKRVESKYGPPCPPCPAPEFLGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGL |
| | | PSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLG |
| 16 | LC | QGQSGQGTSYCSIEHYPCNTHHGGGSSGGSISSGLLSGRSDN |
| | | PGGGSDIQLTQSPSSLSASVGDRVTITCRASESVDAYGISFM |
| | | NWFQQKPGKAPKLLIYAASNQGSGVPSRFSGSGSGTDFTLTI |
| | | SSMQPEDFATYYCQQSKDVPWTFGQGTKLEIKRTVAAPSVF |
| | | IFPPSDEQLKSGTASVVCLLNNFYPREAKVQWKVDNALQSG |
| | | NSQESVTEQDSKDSTYSLSSTLTLSKADYEKHKVYACEVTH |
| | | QGLSSPVTKSFNRGEC |
| 17 | Substrate | LSGRSDNH |
| 18 | Substrate | TGRGPSWV |
| 19 | Substrate | PLTGRSGG |
| 20 | Substrate | TARGPSFK |
| 21 | Substrate | NTLSGRSENHSG |
| 22 | Substrate | NTLSGRSGNHGS |
| 23 | Substrate | TSTSGRSANPRG |
| 24 | Substrate | TSGRSANP |
| 25 | Substrate | VHMPLGFLGP |
| 26 | Substrate | AVGLLAPP |
| 27 | Substrate | AQNLLGMV |
| 28 | Substrate | QNQALRMA |
| 29 | Substrate | LAAPLGLL |
| 30 | Substrate | STFPFGMF |
| 31 | Substrate | ISSGLLSS |
| 32 | Substrate | PAGLWLDP |
| 33 | Substrate | VAGRSMRP |
| 34 | Substrate | VVPEGRRS |
| 35 | Substrate | ILPRSPAF |
| 36 | Substrate | MVLGRSLL |
| 37 | Substrate | QGRAITFI |
| 38 | Substrate | SPRSIMLA |
| 39 | Substrate | SMLRSMPL |
| 40 | Substrate | ISSGLLSGRSDNH |
| 41 | Substrate | AVGLLAPPGGLSGRSDNH |
| 42 | Substrate | ISSGLLSSGGSGGSLSGRSDNH |
| 43 | Substrate | LSGRSGNH |
| 44 | Substrate | SGRSANPRG |
| 45 | Substrate | LSGRSDDH |
| 46 | Substrate | LSGRSDIH |
| 47 | Substrate | LSGRSDQH |
| 48 | Substrate | LSGRSDTH |
| 49 | Substrate | LSGRSDYH |
| 50 | Substrate | LSGRSDNP |
| 51 | Substrate | LSGRSANP |
| 52 | Substrate | LSGRSANI |
| 53 | Substrate | LSGRSDNI |
| 54 | Substrate | MIAPVAYR |
| 55 | Substrate | RPSPMWAY |
| 56 | Substrate | WATPRPMR |
| 57 | Substrate | FRLLDWQW |
| 58 | Substrate | ISSGL |
| 59 | Substrate | ISSGLLS |
| 60 | Substrate | ISSGLL |
| 61 | Substrate | ISSGLLSGRSANPRG |
| 62 | Substrate | AVGLLAPPTSGRSANPRG |
| 63 | Substrate | AVGLLAPPSGRSANPRG |
| 64 | Substrate | ISSGLLSGRSDDH |
| 65 | Substrate | ISSGLLSGRSDIH |
| 66 | Substrate | ISSGLLSGRSDQH |
| 67 | Substrate | ISSGLLSGRSDTH |
| 68 | Substrate | ISSGLLSGRSDYH |
| 69 | Substrate | ISSGLLSGRSDNP |
| 70 | Substrate | ISSGLLSGRSANP |
| 71 | Substrate | ISSGLLSGRSANI |
| 72 | Substrate | AVGLLAPPGGLSGRSDDH |
| 73 | Substrate | AVGLLAPPGGLSGRSDIH |
| 74 | Substrate | AVGLLAPPGGLSGRSDQH |
| 75 | Substrate | AVGLLAPPGGLSGRSDTH |
| 76 | Substrate | AVGLLAPPGGLSGRSDYH |
| 77 | Substrate | AVGLLAPPGGLSGRSDNP |
| 78 | Substrate | AVGLLAPPGGLSGRSANP |
| 79 | Substrate | AVGLLAPPGGLSGRSANI |
| 80 | Substrate | ISSGLLSGRSDNI |
| 81 | Substrate | AVGLLAPPGGLSGRSDNI |
| 82 | Substrate | GLSGRSDNHGGAVGLLAPP |
| 83 | Substrate | GLSGRSDNHGGVHMPLGFLGP |
| 84 | Substrate | PLGL |
| 85 | ProC732 | QSGQTDVDYYREWSWTQVSGSSGGSLSGRSDNIGSGGSCD |
| | | LPQTHSLGSRRTLMLLAQMRRISLFSCLKDRHDFGFPQEEFG |
| | | NQFQKAETIPVLHEMIQQIFNLFSTKDSSAAWDETLLDKFYT |
| | | ELYQQLNDLEACVIQGVGVTETPLMKEDSILAVRKYFQRIT |
| | | LYLKEKKYSPCAWEVVRAEIMRSFSLSTNLQESLRSKELSG |
| | | RSDNICPPCPAPEFLGGPSVFLFPPKPKDTLMISRTPEVTCVV |
| | | VDVSQEDPEVQFNWYVDGVEVHNAKTKPREEQFNSTYRV |
| | | VSVLTVLHQDWLNGKEYKCKVSNKGLPSSIEKTISKAKGQP |
| | | REPQVYTLPPSQEEMTKNQVSLTCLVKGFYPSDIAVEWESN |
| | | GQPENNYKTTPPVLDSDGSFFLYSRLTVDKSRWQQGNVFSC |
| | | SVMHEALHNHYTQKSLSLS |
| 86 | Masking | TDVDYYREWSWTQVS |
| | moiety | |
| 87 | Masking | CDLPQTHSLGSRRTLMLLAQMRRISLFSCLKDRHDFGFPQEE |
| | moiety | FGNQFQKAETIPVLHEMIQQIFNLFSTKDSSAAWDETLLDKF |
| | | YTELYQQLNDLEACVIQGVGVTETPLMKEDSILAVRKYFQR |
| | | ITLYLKEKKYSPCAWEVVRAEIMRS |
| 88 | Masking | CDLPQTHSLGSRRTLMLLAQMRRISLFSCLKDRHDFGFPQEE |
| | moiety | FGNQFQKAETIPVLHEMIQQIFNLFSTKDSSAAWDETLLDKF |
| | | YTELYQQLNDLEACVIQGVGVTETPLMKEDSILAVRKYFQR |
| | | ITLYPKEKKYSPCAWEVVRAEIMRSFSLSTNLQESLRSKE |
| 89 | Masking | IAYLEYYEHLHMAYG |
| | moiety | |
| 90 | Masking | TDVDYYREWCWTQVS |
| | moiety | |
| 91 | Masking | FPLNTFDLVHELLSR |
| | moiety | |
| 92 | Masking | FLNDIHRFLHWTDLM |
| | moiety | |
| 93 | Masking | PYTFVEQVEYWLHAT |
| | moiety | |
| 94 | Masking | ACVIHFLDRISNILE |
| | moiety | |
| 95 | Masking | FCYIAAFSAMQRQSC |
| | moiety | |
| 96 | Masking | PLYLPEIGWMFGLPT |
| | moiety | |
| 97 | Masking | TVLVIPDLHYLYVDR |
| | moiety | |
| 98 | Masking | FINNVETALDTIYNL |
| | moiety | |
| 99 | Masking | SAKHLHPGRLPPMTK |
| | moiety | |
| 100 | Masking | ATMYAYLERLEAILS |
| | moiety | |
| 101 | Masking | IYPLDALLRHLNSLC |
| | moiety | |
| 102 | Masking | CFPTVVWRELYNLYG |
| | moiety | |
| 103 | Masking | NLDFYLNHLYNTLAG |
| | moiety | |
| 104 | Masking | DFINSMRSHLQSSDQ |
| | moiety | |
| 105 | Masking | EPKCSFCSPLIVPSP |
| | moiety | |
| 106 | Masking | PNCIESFLSSIHDSL |
| | moiety | |
| 107 | Masking | TDNALFLETVQHYLY |
| | moiety | |
| 108 | Masking | CYPSISWLFADAPRN |
| | moiety | |
| 109 | Masking | ELTQLLNALVDVRNC |
| | moiety | |
| 110 | Masking | LLSSFVETMSSILTC |
| | moiety | |
| 111 | Masking | YLLRLPSLEELWGPS |
| | moiety | |
| 112 | Masking | ATCYIINHWVERYII |
| | moiety | |
| 113 | Masking | IAYLEYYEHLHMAY |
| | moiety | |
| 114 | Masking | RVTCDDYYYGFGCNKFGRPA |
| | moiety | |
| 115 | Masking | MLAVVGAAALVLVAGAPWVLPSAAGGENLKPPENIDVYII |
| | moiety | DDNYTLKWSSHGESMGSVTFSAEYRTKDEAKWLKVPECQ |
| | | HTTTTKCEFSLLDTNVYIKTQFRVRAEEGNSTSSWNEVDPFI |
| | | PFYTAHMSPPEVRLEAEDKAILVHISPPGQDGNMWALEKPS |
| | | FSYTIRIWQKSSSDKKTINSTYYVEKIPELLPETTYCLEVKAI |
| | | HPSLKKHSNYSTVQCISTTVANKMPVPGNLQVDAQGKSYV |
| | | LKWDYIASADVLFRAQWLPGYSKSSSGSRSDKWKPIPTCAN |
| | | VQTTHCVFSQDTVYTGTFFLHVQASEGNHTSFWSEEKFIDS |
| | | QKHILPPPPVITVTAMSDTLLVYVNCQDSTCDGLNYEIIFWE |
| | | NTSNTKISMEKDGPEFTLKNLQPLTVYCVQARVLFRALLNK |
| | | TSNFSEKLCEKTRPGSFST |
| 116 | Masking | MLLSQNAFIFRSLNLVLMVYISLVFGISYDSPDYTDESCTFKI |
| | moiety | SLRNFRSILSWELKNHSIVPTHYTLLYTIMSKPEDLKVVKNC |
| | | ANTTRSFCDLTDEWRSTHEAYVTVLEGFSGNTTLFSCSHNF |
| | | WLAIDMSFEPPEFEIVGFTNHINVMVKFPSIVEEELQFDLSLV |
| | | IEEQSEGIVKKHKPEIKGNMSGNFTYIIDKLIPNTNYCVSVYL |
| | | EHSDEQAVIKSPLKCTLLPPGQESESAESAK |
| 117 | Masking | MMVVLLGATTLVLVAVAPWVLSAAAGGKNLKSPQKVEVD |
| | moiety | IIDDNFILRWNRSDESVGNVTFSFDYQKTGMDNWIKLSGCQ |
| | | NITSTKCNFSSLKLNVYEEIKLRIRAEKENTSSWYEVDSFTPF |
| | | RKAQIGPPEVHLEAEDKAIVIHISPGTKDSVMWALDGLSFTY |
| | | SLVIWKNSSGVEERIENIYSRHKIYKLSPETTYCLKVKAALL |
| | | TSWKIGVYSPVHCIKTTVENELPPPENIEVSVQNQNYVLKW |
| | | DYTYANMTFQVQWLHAFLKRNPGNHLYKWKQIPDCENVK |
| | | TTQCVFPQNVFQKGIYLLRVQASDGNNTSFWSEEIKFDTEIQ |
| | | AFLLPPVFNIRSLSDSFHIYIGAPKQSGNTPVIQDYPLIYEIIFW |
| | | ENTSNAERKIIEKKTDVTVPNLKPLTVYCVKARAHTMDEKL |
| | | NKSSVFSDAVCEKTKPGNTSK |
| 118 | Masking | MRSRCTVSAVGLLSLCLVVSASLETITPSAFDGYPDEPCTINI |
| | moiety | TIRNSRLILSWELENKSGPPANYTLWYTVMSKDENLTKVKN |
| | | CSDTTKSSCDVTDKWLEGMESYVVAIVIVHRGDLTVCRCSD |
| | | YIVPANAPLEPPEFEIVGFTDHINVTMEFPPVTSKIIQEKMKT |
| | | TPFVIKEQIGDSVRKKHEPKVNNVTGNFTFVLRDLLPKTNY |
| | | CVSLYFDDDPAIKSPLKCIVLQPGQESGLSESA |
| 119 | Masking | TDVDYYREW |
| | moiety | |
| 120 | Masking | GSGTDVDYYREWSWTQV |
| | moiety | |
| 121 | Masking | GSGTDVDYYREWSWTQVS |
| | moiety | |
| 122 | Masking | TDVDYYREWSWTQV |
| | moiety | |
| 123 | Masking | TDVDYYREWSWTQVS |
| | moiety | |
| 124 | Masking | ALTSTEDPEPPSVPVPTNVLIKSYNLNPVVCWEYQNMSQTPI |
| | moiety | FTVQVKVYSGSWTDSCTNISDHCCNIYEQIMYPDVSAWAR |
| | | VKAKVGQKESDYARSKEFLMCLKGKVGPPGLEIRRKKEEQ |
| | | LSVLVFHPEVVVNGESQGTMFGDGSTCYTFDYTVYVEHNR |
| | | SGEILHTKHTVEKEECNETLCELNISVSTLDSRYCLSVDGISS |
| | | FWQVRTEKSKDVCIPPFHDDRKDS |
| 125 | Masking | ASSPDSFSQLAAPLNPRLHLYNDEQILTWEPSPSSNDPRPVV |
| | moiety | YQVEYSFIDGSWHRLLEPNCTDITETKCDLTGGGRLKLFPHP |
| | | FTVFLRVRAKRGNLTSKWVGLEPFQHYENVTVGPPKNISVT |
| | | PGKGSLVIHFSPPFDVFHGATFQYLVHYWEKSETQQEQVEG |
| | | PFKSNSIVLGNLKPYRVYCLQTEAQLILKNKKIRPHGLLSNV |
| | | SCHETTANASARLQQVILIPLGIFALLLGLTGACFTLFLKYQS |
| | | RVKYWFQAPPNIPEQIEEYLKDPDQFILEVLDKDGSPKEDSW |
| | | DSVSIISSPEKERDDVLQTP |
| 126 | Masking | EMGTADLGPSSVPTPTNVTIESYNMNPIVYWEYQIMPQVPV |
| | moiety | FTVEVKNYGVKNSEWIDACINISHHYCNISDHVGDPSNSLW |
| | | VRVKARVGQKESAYAKSEEFAVCRDGKIGPPKLDIRKEEKQ |
| | | IMIDIFHPSVFVNGDEQEVDYDPETTCYIRVYNVYVRMNGS |
| | | EIQYKILTQKEDDCDEIQCQLAIPVSSLNSQYCVSAEGVLHV |
| | | WGVTTEKSKEVCITIFNSSIKG |
| 127 | Masking | SQLPAPQHPKIRLYNAEQVLSWEPVALSNSTRPVVYQVQFK |
| | moiety | YTDSKWFTADIMSIGVNCTQITATECDFTAASPSAGFPMDFN |
| | | VTLRLRAELGALHSAWVTMPWFQHYRNVTVGPPENIEVTP |
| | | GEGSLIIRFSSPFDIADTSTAFFCYYVHYWEKGGIQQVKGPFR |
| | | SNSISLDNLKPSRVYCLQVQAQLLWNKSNIFRVGHLSNISCY |
| | | ETMADASTELQQ |
| 128 | Masking | CISPRG |
| | moiety | |
| 129 | Masking | CISPRGC |
| | moiety | |
| 130 | Masking | CISPRGCG |
| | moiety | |
| 131 | Masking | CISPRGCPDGPYVMY |
| | moiety | |
| 132 | Masking | CISPRGCPDGPYVM |
| | moiety | |
| 133 | Masking | CISPRGCEPGTYVPT |
| | moiety | |
| 134 | Masking | CISPRGCPGQIWHPP |
| | moiety | |
| 135 | Masking | GSHCLIPINMGAPSC |
| | moiety | |
| 136 | Masking | CISPRGCGGSSASQSGQGSHCLIPINMGAPSC |
| | moiety | |
| 137 | Masking | CNHHYFYTCGCISPRGCPG |
| | moiety | |
| 138 | Masking | ADHVFWGSYGCISPRGCPG |
| | moiety | |
| 139 | Masking | CHHVYWGHCGCISPRGCPG |
| | moiety | |
| 140 | Masking | CPHFTTTSCGCISPRGCPG |
| | moiety | |
| 141 | Masking | CNHHYHYYCGCISPRGCPG |
| | moiety | |
| 142 | Masking | CPHVSFGSCGCISPRGCPG |
| | moiety | |
| 143 | Masking | CPYYTLSYCGCISPRGCPG |
| | moiety | |
| 144 | Masking | CNHVYFGTCGCISPRGCPG |
| | moiety | |
| 145 | Masking | CNHFTLTTCGCISPRGCPG |
| | moiety | |
| 146 | Masking | CHHFTLTTCGCISPRGCPG |
| | moiety | |
| 147 | Masking | YNPCATPMCCISPRGCPG |
| | moiety | |
| 148 | Masking | CNHHYFYTCGCISPRGCG |
| | moiety | |
| 149 | Masking | CNHHYHYYCGCISPRGCG |
| | moiety | |
| 150 | Masking | CNHVYFGTCGCISPRGCG |
| | moiety | |
| 151 | Masking | CHHVYWGHCGCISPRGCG |
| | moiety | |
| 152 | Masking | CPHFTTTSCGCISPRGCG |
| | moiety | |
| 153 | Masking | CNHFTLTTCGCISPRGCG |
| | moiety | |
| 154 | Masking | CHHFTLTTCGCISPRGCG |
| | moiety | |
| 155 | Masking | CPYYTLSYCGCISPRGCG |
| | moiety | |
| 156 | Masking | CPHVSFGSCGCISPRGCG |
| | moiety | |
| 157 | Masking | ADHVFWGSYGCISPRGCG |
| | moiety | |
| 158 | Masking | YNPCATPMCCISPRGCG |
| | moiety | |
| 159 | Masking | CHHVYWGHCGCISPRGCG |
| | moiety | |
| 160 | Masking | CISPRGCGQPIPSVK |
| | moiety | |
| 161 | Masking | CISPRGCTQPYHVSR |
| | moiety | |
| 162 | Masking | CISPRGCNAVSGLGS |
| | moiety | |

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and scope of the appended claims.

What is claimed is:

1. A method of determining the level of protease activity in a biological sample, the method comprising:
(a) contacting a biological sample with a solution comprising a QZ probe to form a mixture, wherein the QZ probe comprises at least one bipartite polypeptide having a component A, a CL, and a component B in a structural arrangement of, from N-terminus to C-terminus,
A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide comprising the component A or a portion thereof, and a cleaved polypeptide comprising the component B or a portion thereof,
(b) incubating the mixture, thereby forming an incubated mixture comprising an incubated liquid; and
(c) measuring the quantity of one or more analytes in a sample of the incubated liquid to determine the level of protease activity in the biological sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

2. The method of claim 1, wherein the biological samples is a cell.

3. The method of claim 1, wherein the biological sample is an organoid.

4. A method of determining the level of protease activity in a biological sample, the method comprising:
(a) contacting a biological sample with a solution comprising a QZ probe to form a mixture, wherein the biological sample is a tissue sample, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide that comprises component A or a portion thereof, and a cleaved polypeptide comprising component B or a portion thereof,
(b) incubating the mixture, thereby forming an incubated mixture comprising the tissue sample and an incubated liquid; and
(c) measuring the quantity of one or more analytes in a sample of the incubated liquid to determine the level of protease activity in the tissue sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

5. A method of determining the level of protease activity in a biological sample comprising a liquid, the method comprising:
(a) contacting a biological sample with a QZ probe to form a mixture, wherein the biological sample comprises a liquid, and wherein the QZ probe comprises at least one bipartite polypeptide having a component A, a CL, and a component B in a structural arrangement of, from N-terminus to C-terminus, A-CL-B or B-CL-A,
wherein the component A and the component B are each independently a polypeptide,
wherein CL is a cleavable linker comprising a substrate for a protease,
wherein cleavage of the CL generates a cleavage product comprising a cleaved polypeptide comprising one or more cleaved polypeptide species comprising the component A or a portion thereof and a cleaved polypeptide comprising the component B or a portion thereof,
(b) incubating the mixture thereby forming an incubated mixture comprising an incubated liquid; and
(c) measuring the quantity of one or more analytes in a sample of incubated liquid to determine the level of protease activity in the biological sample, wherein the analyte is selected from the group consisting of the cleaved polypeptide comprising the component A or portion thereof, the cleaved polypeptide comprising the component B or portion thereof, an uncleaved bipartite polypeptide, and any combination of two or more thereof.

6. The method of claim 5, wherein the biological sample is selected from the group consisting of a cell culture supernatant, a cell lysate supernatant, an organoid culture supernatant, blood, bile, bone marrow aspirate, breast milk, cerebrospinal fluid, plasma, saliva, serum, sputum, synovial fluid, and urine.

7. The method of claim 5, wherein the biological sample is plasma.

8. The method of any one of claims 1-7, wherein the bipartite polypeptide comprises the structure of, from N-terminus to C-terminus, B-CL-A.

9. The method of any one of claims 1-8, wherein the QZ probe comprises a polypeptide complex comprising one or more further polypeptides.

10. The method of any one of claims 1-9, wherein the QZ probe comprises an antibody, wherein at least one of the components A and B of the bipartite polypeptide comprises an antibody domain selected from the group consisting of a light chain variable domain, a heavy chain variable domain, and a combination thereof.

11. The method of claim 10, wherein the QZ probe comprises an antibody comprising a first polypeptide, a second polypeptide, a third polypeptide, and a fourth polypeptide, wherein:
(1) the first polypeptide comprises a bipartite polypeptide comprising a first component A that comprises a first light chain variable domain;
(2) the second polypeptide comprises a second component A comprising a second light chain variable domain;
(3) the third polypeptide comprises a first heavy chain variable domain; and
(4) the fourth polypeptide comprises a second heavy chain variable domain.

12. The method of claim 10, wherein the QZ probe comprises an antibody comprising a first polypeptide, a second polypeptide, a third polypeptide, and a fourth polypeptide, wherein: (1) the first polypeptide comprises a bipartite polypeptide comprising a first component A that comprises a first heavy chain variable domain, (2) the second polypeptide comprises a second component A comprising a second heavy chain variable domain; (3) the third polypeptide comprises a first light chain variable domain; and (4) the fourth polypeptide comprises a second light chain variable domain.

13. The method of any one of claims 1-12, wherein each component B comprises a polypeptide having at least 3 amino acid residues.

14. The method of any one of claims 1-9, wherein the QZ probe comprises an activatable antibody and each component B comprises a masking moiety.

15. The method of claim 14, wherein the QZ probe comprises an activatable antibody comprising a first, a second, a third, and a fourth polypeptide, wherein:
the first polypeptide is a first light chain comprising a first bipartite polypeptide, wherein the component A comprises a first light chain variable domain, and a light chain constant domain, and the component B comprises a first masking moiety;
the second polypeptide is a second light chain comprising a second bipartite polypeptide, wherein the component A comprises a second light chain variable domain, and a light chain constant domain, and the component B comprises a second masking moiety;
the third polypeptide is a first heavy chain comprising a first heavy chain variable domain, and a CH1, a CH2, a CH3, a hinge region, and an Fc domain; and
the fourth polypeptide is a second heavy chain comprising a second heavy chain variable domain, and a CH1, a CH2, a CH3, a hinge region, and an Fc domain.

16. The method of claim 14, wherein the QZ probe comprises an activatable antibody comprising a first, a second, a third, and a fourth polypeptide, wherein:
the first polypeptide is a first light chain comprising a first light chain variable domain, and a light chain constant domain;
the second polypeptide is a second light chain comprising a second light chain variable domain and a light chain constant domain;
the third polypeptide is a first heavy chain comprising a first bipartite polypeptide, wherein the component A comprises a first heavy chain variable domain and a CH1, a CH2, a CH3, a hinge region, and an Fc domain, and the component B comprises a first masking moiety; and
the fourth polypeptide is a second heavy chain comprising a bipartite polypeptide, wherein the component A comprises a second heavy chain variable domain, and a CH1, a CH2, a CH3, a hinge region, and an Fc domain, and the component B comprises a second masking moiety.

17. The method of any one of claims 1-9, wherein the QZ probe comprises a pseudo-antibody, wherein one of the component A and the component B comprises a pseudo-antibody variable domain selected from the group consisting of a pseudo-light chain variable domain and a pseudo-heavy chain variable domain, and a combination thereof.

18. The method of any one of claims 1-9, wherein the QZ probe comprises a pseudo-antibody comprising the bipartite polypeptide and a second polypeptide, wherein either
(i) the component A comprises a pseudo-light chain variable domain and the second polypeptide comprises a domain selected from the group consisting of a heavy chain variable domain and a pseudo-heavy chain variable domain; or
(ii) the component A comprises a pseudo-heavy chain variable domain and the second polypeptide comprises a domain selected from the group consisting of a light chain variable domain and a pseudo-light chain variable domain.

19. The method of any one of claims 1-9, wherein the QZ probe comprises a pseudo-antibody comprising the bipartite polypeptide and a second polypeptide, wherein either
(i) the component A comprises a light chain variable domain and the second polypeptide comprises a pseudo-heavy chain domain; or
(ii) the component A comprises a heavy chain variable domain and the second polypeptide comprises a pseudo-light chain variable domain.

20. The method of any one of claims 1-9, wherein the QZ probe comprises a pseudo-antibody comprising a first polypeptide, a second polypeptide, a third polypeptide, and a fourth polypeptide, wherein:
(1) the first polypeptide is a light chain comprising a bipartite polypeptide, wherein the component A comprises a domain selected from the group consisting of a first light chain variable domain and a first pseudo-light chain variable domain;
(2) the second polypeptide is a light chain comprising a bipartite polypeptide, wherein the component A comprises a domain selected from the group consisting of a second light chain variable domain and a second pseudo-light chain variable domain;
(3) the third polypeptide is a heavy chain comprising a first pseudo-heavy chain variable domain; and
(4) the fourth polypeptide is a heavy chain comprising a second pseudo-heavy chain variable domain.

21. The method of any one of claims 1-9, wherein the QZ probe comprises a pseudo-antibody comprising a first polypeptide, a second polypeptide, a third polypeptide, and a fourth polypeptide, wherein (1) the first polypeptide is a light chain comprising a first bipartite polypeptide, wherein the component A comprises a first pseudo-light chain variable domain; (2) the second polypeptide is a light chain comprising a second bipartite polypeptide, wherein the component A comprises a second pseudo-light chain variable domain; (3) the third polypeptide is a heavy chain comprising a domain selected from the group consisting of a first heavy chain variable domain and a first pseudo-heavy chain variable domain; and (4) the fourth polypeptide is a heavy chain comprising a domain selected from the group consisting of a second heavy chain variable domain and a second pseudo-heavy chain variable domain.

22. The method of any one of claims 17-21, wherein each component B is a polypeptide comprising at least about 3 amino acid residues.

23. The method of any one of claims 17-21, wherein the pseudo-antibody is a variant of a parental antibody and wherein each component B comprises a parental masking moiety.

24. The method of any one of claims 1-9, wherein the QZ probe comprises an activatable pseudo-antibody comprising a first polypeptide, a second polypeptide, a third polypeptide, and a fourth polypeptide,
wherein:
the first polypeptide is a first light chain comprising a first bipartite polypeptide, wherein the component A comprises (i) a first domain selected from the group consisting of first pseudo-light chain variable domain and a first light chain variable domain, and (ii) a light chain constant domain, and the component B comprises a first parental masking moiety;
the second polypeptide is a second light chain comprising a second bipartite polypeptide, wherein the component A comprises (i) a second domain selected from the group consisting of a second pseudo-light chain variable domain and a second light chain variable domain, and (ii) a light chain constant domain, and the component B comprises a second parental masking moiety;
the third polypeptide is a heavy chain comprising (i) a third domain selected from the group consisting of a first pseudo-heavy chain domain and a first heavy chain variable domain, and (ii) a CH1, a CH2, a CH3, a hinge region, and an Fc domain; and
the fourth polypeptide is a heavy chain comprising (i) fourth domain selected from the group consisting of a second pseudo-heavy chain domain and a second heavy chain variable domain, and (ii) a CH1, a CH2, a CH3, a hinge region, and an Fc domain,
with the proviso that at least one of the first domain, the second domain, the third domain, and the fourth domain is a pseudo-variable domain.

25. The method of any one of claims 1-9, wherein the QZ probe comprises an activatable pseudo-antibody comprising a first polypeptide, a second polypeptide, a third polypeptide, and a fourth polypeptide, wherein:
the first polypeptide is a first light chain comprising (i) a domain selected from the group consisting of first pseudo-light chain variable domain and a first light chain variable domain, and (ii) a light chain constant domain;
the second polypeptide is a second light chain comprising (i) a domain selected from the group consisting of a second pseudo-light chain variable domain and a second light chain variable domain, and (ii) a light chain constant domain;
the third polypeptide is a heavy chain comprising a first bipartite polypeptide, wherein the component A comprises (i) a domain selected from the group consisting of a first pseudo-heavy chain variable domain and a first heavy chain variable domain and (ii) a CH1, a CH2, a CH3, a hinge region, and an Fc domain, and the component B comprises a first parental masking moiety; and
the fourth polypeptide is a heavy chain comprising a bipartite polypeptide, wherein the component A comprises (i) a domain selected from the group consisting of a second pseudo-heavy chain domain and a second heavy chain variable domain, and (ii) a CH1, a CH2, a CH3, a hinge region, and an Fc domain, and the component B comprises a second parental masking moiety,
with the proviso that at least one of the first domain, the second domain, the third domain, and the fourth domain is a pseudo-antibody variable domain.

26. The method of any one of claims 1-25, further comprising performing a plurality of cycles of steps (a)-(c).

27. The method of claim 26, wherein the CL in each of the plurality of cycles or subset thereof, is different.

28. The method of any one of claims 26-27, wherein the component A in each of the plurality of cycles or subset thereof, is the same.

29. The method of any one of claims 26-27, wherein the component B in each of the plurality of cycles or subset thereof, is the same.

30. The method of any one of claims 26-27, wherein the component A in each of the plurality of cycles or subset thereof, is the same, and the component B in each of the plurality of cycles or subset thereof, is the same.

31. The method of any one of claims 1-30, further comprising performing a plurality of cycles of steps (a)-(c), wherein in each cycle, the biological sample is incubated with one or more protease inhibitors or a combination of two or more protease inhibitors prior to step (a) and/or during step (a).

32. The method of claim 31, wherein the protease inhibitor in each cycle of the plurality of cycles is different.

33. The method of any one of claims 31-32, wherein the QZ probe in each cycle of the plurality of cycles is the same.

34. The method of any one of claims 26-33, wherein the plurality of cycles is performed in parallel.

35. The method of claim 34, wherein the plurality of cycles is performed in a multi-well plate.

36. The method of any one of claims 26-33, wherein the plurality of cycles is performed in series.

37. The method of any one of claims 1-36, wherein the QZ probe comprises a plurality of distinct species of QZ probes, and wherein the biological sample is contacted with the plurality of distinct QZ probes.

38. The method of claim 37, wherein each distinct species of QZ probe in the plurality or subset thereof comprises a CL having a different substrate.

39. The method of any one of claims 37-38, wherein measuring the quantity of one or more analytes in a sample of the incubated liquid comprises measuring a distinct signal associated with each distinct species of QZ probe in the plurality.

40. The method of any one of claims 1-39, wherein each CL comprises a substrate for a protease selected from the group consisting of a disintegrin and metalloprotease (ADAM), a disintegrin and metalloproteinase with thrombospondin motifs (ADAMTS), an aspartate protease, an aspartic cathepsin, a caspase, a cysteine cathepsin, a cysteine proteinase, a KLK, a metalloproteinase, a matrix metalloproteinase (MMP), a serine protease, a coagulation factor protease, and a Type II Transmembrane Serine Protease (TTSP).

41. The method of any one of claims 1-39, wherein each CL comprises a substrate for at least one protease selected from the group consisting of ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathepsin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP1, MMP2, MMP3, MMP7, MMP8, MMP9, MMP10, MMP11, MMP12, MMP13, MMP14, MMPP15, MMPP16, MMP17, MMP19, MMP20, MMP23, MMP24, MMP26, MMP27, activated protein C, Cathepsin A, Cathepsin G, Chymase, FVIIa, FIXa, FXa, FXI, FXIIa, elastase, granzyme B, Guanidinobenzoatase, HtrA1, proteinase 3, neutrophil elastase, NSP4, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matriptase, TMPRSS2, TMPRSS3, TMPRSS4, TMPRSS5, TMPRSS6, TMPRSS7, TMPRSS8, TMPRSS9, TMPRSS10, and TMPRSS11.

42. The method of any one of claims 1-39, wherein each CL comprises a substrate for a serine protease.

43. The method of any one of claims 1-39, wherein each CL comprises a substrate for a matrix metalloproteinase (MMP).

44. The method of any one of claims 1-39, wherein each CL comprises a substrate for an aspartate protease, cysteine protease, or threonine protease.

45. The method of any one of claims 1-39, wherein each CL comprises a substrate having an amino acid sequence selected from the group consisting of SEQ ID NOs: 17-84.

46. The method of any one of claims 1-45, wherein the one or more measured analytes comprise an uncleaved bipartite polypeptide.

47. The method of any one of claims 1-45, wherein the one or more measured analytes comprise one or more species of cleaved polypeptide comprising the component A or portion thereof and the uncleaved bipartite polypeptide.

48. The method of any one of claims 1-45, wherein the one or more measured analytes comprise one or more species of the cleaved polypeptide comprising the component B or portion thereof and the uncleaved bipartite polypeptide.

49. The method of any one of claims 1-45, wherein the one or more measured analytes comprise one or more species of the cleaved polypeptide comprising the component A or portion thereof, one or more species of the cleaved polypeptide comprising the component B or portion thereof, and the uncleaved bipartite polypeptide.

50. A method of identifying a patient suitable for a treatment with a protease-activatable therapeutic molecule, the method comprising:
determining the level of protease activity in a biological sample from the patient according to the method of any one of claims 1-49,
wherein the protease-activatable therapeutic molecule is activated by a target protease, and
wherein, if the biological sample is determined to have target-protease activity, then the patient is identified as being suitable for treatment with a protease-activatable therapeutic molecule.

51. A method of treating a patient having a disease or disorder with a protease-activatable therapeutic molecule that is activated by a target protease, the method comprising:
administering to a patient having a disease or disorder a therapeutically effective amount of a protease-activatable therapeutic molecule,
wherein the patient has been identified as suitable for treatment with the protease-activatable therapeutic molecule in accordance with the method of claim 50.

52. The method of any one of claims 50-51, wherein the patient has a disorder or a disease selected from the group consisting of a cardiovascular disease, a neoplastic disease, a neurodegenerative disease, an inflammatory disease, a skin disease, an infectious disease, a bacterial infection, a viral infection, an autoimmune disease, a metabolic disease, a hematologic disease, and a cancer.

53. The method of any one of claims 1-9, wherein the QZ probe comprises an activatable cytokine, wherein the component A of the bipartite polypeptide comprises a cytokine and the component B comprises a masking moiety.

54. The method of any one of claims 1-53, wherein each QZ probe further comprises a detectable label.

55. The method of any one of claims 1-54, further comprising measuring the quantity of analyte comprises using a secondary reagent that binds to at least one analyte, wherein the secondary reagent is attached to a detectable label.

56. The method of any one of claims 1-55, wherein the quantities of one or more analytes is/are determined by subjecting the sample(s) of incubated liquid to capillary electrophoresis.

57. The method of claim 56, wherein the capillary electrophoresis is reducing capillary electrophoresis.

58. The method of any one of claims 1-57, wherein the quantities of one or more analytes is/are determined by subjecting the sample(s) of incubated liquid to a capillary electrophoresis immunoassay.


SEQUENCE LISTING (162 sequences)

SEQ ID NO: 1 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
QVQLKQSGPGLVQPSQSLSITCTVSGFSLTSYGVHWVRQSPGKGLEWLGVIWSKGSTAYNTPFTSRLSINKDNSKSQVFFKMNSLQSQDTAIYYCARALTSGKAEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct; identical to SEQ ID NO: 2

SEQ ID NO: 5 - protein, 261 aa, synthetic construct
QGQSGQCISPRGCPDGPYVMYGSSGGSGGSGGSGLSGRSDNHGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 257 aa, synthetic construct
QGQSGQCISPRGCPDGPYVMYGSSGGSGGSGGSGPLGLGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 266 aa, synthetic construct
QGQSGQCISPRGCPDGPYVMYGSSGGSGGSGGSGISSGLLSGRSDNHGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 271 aa, synthetic construct
QGQSGQCISPRGCPDGPYVMYGSSGGSGGSGGSGAVGLLAPPGGLSGRSDNHGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 261 aa, synthetic construct
QGQSGQCISPRGCPDGPYVMYGSSGGSGGSGGSGISSGLLSSGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 260 aa, synthetic construct
QGQSGQCISPRGCPDGPYVMYGSSGGSGGSGGSGLSGRSDNHGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 11 - protein, 266 aa, synthetic construct; identical to SEQ ID NO: 7

SEQ ID NO: 12 - protein, 261 aa, synthetic construct
QGQSGQCISPRGCPDGPYVMYGSSGGSGGSGGSGAVGLLAPPGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 261 aa, synthetic construct; identical to SEQ ID NO: 5

SEQ ID NO: 14 - protein, 271 aa, synthetic construct; identical to SEQ ID NO: 8

SEQ ID NO: 15 - protein, 445 aa, synthetic construct
EVQLVESGGGLVQPGGSLRLSCAASGFTFSGYAMSWVRQAPGKGLEWVAYISNSGGNAHYADSVKGRFTISRDNSKNTLYLQMNSLRAEDTAVYYCTREDYGTSPFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 265 aa, synthetic construct
QGQSGQGTSYCSIEHYPCNTHHGGGSSGGSISSGLLSGRSDNPGGGSDIQLTQSPSSLSASVGDRVTITCRASESVDAYGISFMNWFQQKPGKAPKLLIYAASNQGSGVPSRFSGSGSGTDFTLTISSMQPEDFATYYCQQSKDVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 8 aa, synthetic construct
LSGRSDNH

SEQ ID NO: 18 - protein, 8 aa, synthetic construct
TGRGPSWV

SEQ ID NO: 19 - protein, 8 aa, synthetic construct
PLTGRSGG

SEQ ID NO: 20 - protein, 8 aa, synthetic construct
TARGPSFK

SEQ ID NO: 21 - protein, 12 aa, synthetic construct
NTLSGRSENHSG

SEQ ID NO: 22 - protein, 12 aa, synthetic construct
NTLSGRSGNHGS

SEQ ID NO: 23 - protein, 12 aa, synthetic construct
TSTSGRSANPRG

SEQ ID NO: 24 - protein, 8 aa, synthetic construct
TSGRSANP

SEQ ID NO: 25 - protein, 10 aa, synthetic construct
VHMPLGFLGP

SEQ ID NO: 26 - protein, 8 aa, synthetic construct
AVGLLAPP

SEQ ID NO: 27 - protein, 8 aa, synthetic construct
AQNLLGMV

SEQ ID NO: 28 - protein, 8 aa, synthetic construct
QNQALRMA

SEQ ID NO: 29 - protein, 8 aa, synthetic construct
LAAPLGLL

SEQ ID NO: 30 - protein, 8 aa, synthetic construct
STFPFGMF

SEQ ID NO: 31 - protein, 8 aa, synthetic construct
ISSGLLSS

SEQ ID NO: 32 - protein, 8 aa, synthetic construct
PAGLWLDP

SEQ ID NO: 33 - protein, 8 aa, synthetic construct
VAGRSMRP

SEQ ID NO: 34 - protein, 8 aa, synthetic construct
VVPEGRRS

SEQ ID NO: 35 - protein, 8 aa, synthetic construct
ILPRSPAF

SEQ ID NO: 36 - protein, 8 aa, synthetic construct
MVLGRSLL

SEQ ID NO: 37 - protein, 8 aa, synthetic construct
QGRAITFI

SEQ ID NO: 38 - protein, 8 aa, synthetic construct
SPRSIMLA

SEQ ID NO: 39 - protein, 8 aa, synthetic construct
SMLRSMPL

SEQ ID NO: 40 - protein, 13 aa, synthetic construct
ISSGLLSGRSDNH

SEQ ID NO: 41 - protein, 18 aa, synthetic construct
AVGLLAPPGGLSGRSDNH

SEQ ID NO: 42 - protein, 22 aa, synthetic construct
ISSGLLSSGGSGGSLSGRSDNH

SEQ ID NO: 43 - protein, 8 aa, synthetic construct
LSGRSGNH

SEQ ID NO: 44 - protein, 9 aa, synthetic construct
SGRSANPRG

SEQ ID NO: 45 - protein, 8 aa, synthetic construct
LSGRSDDH

SEQ ID NO: 46 - protein, 8 aa, synthetic construct
LSGRSDIH

SEQ ID NO: 47 - protein, 8 aa, synthetic construct
LSGRSDQH

SEQ ID NO: 48 - protein, 8 aa, synthetic construct
LSGRSDTH

SEQ ID NO: 49 - protein, 8 aa, synthetic construct
LSGRSDYH

SEQ ID NO: 50 - protein, 8 aa, synthetic construct
LSGRSDNP

SEQ ID NO: 51 - protein, 8 aa, synthetic construct
LSGRSANP

SEQ ID NO: 52 - protein, 8 aa, synthetic construct
LSGRSANI

SEQ ID NO: 53 - protein, 8 aa, synthetic construct
LSGRSDNI

SEQ ID NO: 54 - protein, 8 aa, synthetic construct
MIAPVAYR

SEQ ID NO: 55 - protein, 8 aa, synthetic construct
RPSPMWAY

SEQ ID NO: 56 - protein, 8 aa, synthetic construct
WATPRPMR

SEQ ID NO: 57 - protein, 8 aa, synthetic construct
FRLLDWQW

SEQ ID NO: 58 - protein, 5 aa, synthetic construct
ISSGL

SEQ ID NO: 59 - protein, 7 aa, synthetic construct
ISSGLLS

SEQ ID NO: 60 - protein, 6 aa, synthetic construct
ISSGLL

SEQ ID NO: 61 - protein, 15 aa, synthetic construct
ISSGLLSGRSANPRG

SEQ ID NO: 62 - protein, 18 aa, synthetic construct
AVGLLAPPTSGRSANPRG

SEQ ID NO: 63 - protein, 17 aa, synthetic construct
AVGLLAPPSGRSANPRG

SEQ ID NO: 64 - protein, 13 aa, synthetic construct
ISSGLLSGRSDDH

SEQ ID NO: 65 - protein, 13 aa, synthetic construct
ISSGLLSGRSDIH

SEQ ID NO: 66 - protein, 13 aa, synthetic construct
ISSGLLSGRSDQH

SEQ ID NO: 67 - protein, 13 aa, synthetic construct
ISSGLLSGRSDTH

SEQ ID NO: 68 - protein, 13 aa, synthetic construct
ISSGLLSGRSDYH

SEQ ID NO: 69 - protein, 13 aa, synthetic construct
ISSGLLSGRSDNP

SEQ ID NO: 70 - protein, 13 aa, synthetic construct
ISSGLLSGRSANP

SEQ ID NO: 71 - protein, 13 aa, synthetic construct
ISSGLLSGRSANI

SEQ ID NO: 72 - protein, 18 aa, synthetic construct
AVGLLAPPGGLSGRSDDH

SEQ ID NO: 73 - protein, 18 aa, synthetic construct
AVGLLAPPGGLSGRSDIH

SEQ ID NO: 74 - protein, 18 aa, synthetic construct
AVGLLAPPGGLSGRSDQH

SEQ ID NO: 75 - protein, 18 aa, synthetic construct
AVGLLAPPGGLSGRSDTH

SEQ ID NO: 76 - protein, 18 aa, synthetic construct
AVGLLAPPGGLSGRSDYH

SEQ ID NO: 77 - protein, 18 aa, synthetic construct
AVGLLAPPGGLSGRSDNP

SEQ ID NO: 78 - protein, 18 aa, synthetic construct
AVGLLAPPGGLSGRSANP

SEQ ID NO: 79 - protein, 18 aa, synthetic construct
AVGLLAPPGGLSGRSANI

SEQ ID NO: 80 - protein, 13 aa, synthetic construct
ISSGLLSGRSDNI

SEQ ID NO: 81 - protein, 18 aa, synthetic construct
AVGLLAPPGGLSGRSDNI

SEQ ID NO: 82 - protein, 19 aa, synthetic construct
GLSGRSDNHGGAVGLLAPP

SEQ ID NO: 83 - protein, 21 aa, synthetic construct
GLSGRSDNHGGVHMPLGFLGP

SEQ ID NO: 84 - protein, 4 aa, synthetic construct
PLGL

SEQ ID NO: 85 - protein, 430 aa, synthetic construct
QSGQTDVDYYREWSWTQVSGSSGGSLSGRSDNIGSGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKELSGRSDNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 86 - protein, 15 aa, synthetic construct
TDVDYYREWSWTQVS

SEQ ID NO: 87 - protein, 150 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRS

SEQ ID NO: 88 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYPKEKKYSPCAWEVVRAEIMRSFSLSTNLQESLRSKE

SEQ ID NO: 89 - protein, 15 aa, synthetic construct
IAYLEYYEHLHMAYG

SEQ ID NO: 90 - protein, 15 aa, synthetic construct
TDVDYYREWCWTQVS

SEQ ID NO: 91 - protein, 15 aa, synthetic construct
FPLNTFDLVHELLSR

SEQ ID NO: 92 - protein, 15 aa, synthetic construct
FLNDIHRFLHWTDLM

SEQ ID NO: 93 - protein, 15 aa, synthetic construct
PYTFVEQVEYWLHAT

SEQ ID NO: 94 - protein, 15 aa, synthetic construct
ACVIHFLDRISNILE

SEQ ID NO: 95 - protein, 15 aa, synthetic construct
FCYIAAFSAMQRQSC

SEQ ID NO: 96 - protein, 15 aa, synthetic construct
PLYLPEIGWMFGLPT

SEQ ID NO: 97 - protein, 15 aa, synthetic construct
TVLVIPDLHYLYVDR

SEQ ID NO: 98 - protein, 15 aa, synthetic construct
FINNVETALDTIYNL

SEQ ID NO: 99 - protein, 15 aa, synthetic construct
SAKHLHPGRLPPMTK

SEQ ID NO: 100 - protein, 15 aa, synthetic construct
ATMYAYLERLEAILS

SEQ ID NO: 101 - protein, 15 aa, synthetic construct
IYPLDALLRHLNSLC

SEQ ID NO: 102 - protein, 15 aa, synthetic construct
CFPTVVWRELYNLYG

SEQ ID NO: 103 - protein, 15 aa, synthetic construct
NLDFYLNHLYNTLAG

SEQ ID NO: 104 - protein, 15 aa, synthetic construct
DFINSMRSHLQSSDQ

SEQ ID NO: 105 - protein, 15 aa, synthetic construct
EPKCSFCSPLIVPSP

SEQ ID NO: 106 - protein, 15 aa, synthetic construct
PNCIESFLSSIHDSL

SEQ ID NO: 107 - protein, 15 aa, synthetic construct
TDNALFLETVQHYLY

SEQ ID NO: 108 - protein, 15 aa, synthetic construct
CYPSISWLFADAPRN

SEQ ID NO: 109 - protein, 15 aa, synthetic construct
ELTQLLNALVDVRNC

SEQ ID NO: 110 - protein, 15 aa, synthetic construct
LLSSFVETMSSILTC

SEQ ID NO: 111 - protein, 15 aa, synthetic construct
YLLRLPSLEELWGPS

SEQ ID NO: 112 - protein, 15 aa, synthetic construct
ATCYIINHWVERYII

SEQ ID NO: 113 - protein, 14 aa, synthetic construct
IAYLEYYEHLHMAY

SEQ ID NO: 114 - protein, 20 aa, synthetic construct
RVTCDDYYYGFGCNKFGRPA

SEQ ID NO: 115 - protein, 429 aa, synthetic construct
MLAVVGAAALVLVAGAPWVLPSAAGGENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHILPPPPVITVTAMSDTLLVYVNCQDSTCDGLNYEIIFWENTSNTKISMEKDGPEFTLKNLQPLTVYCVQARVLFRALLNKTSNFSEKLCEKTRPGSFST

SEQ ID NO: 116 - protein, 243 aa, synthetic construct
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 117 - protein, 436 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 118 - protein, 242 aa, synthetic construct
MRSRCTVSAVGLLSLCLVVSASLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESA

SEQ ID NO: 119 - protein, 9 aa, synthetic construct
TDVDYYREW

SEQ ID NO: 120 - protein, 17 aa, synthetic construct
GSGTDVDYYREWSWTQV

SEQ ID NO: 121 - protein, 18 aa, synthetic construct
GSGTDVDYYREWSWTQVS

SEQ ID NO: 122 - protein, 14 aa, synthetic construct
TDVDYYREWSWTQV

SEQ ID NO: 123 - protein, 15 aa, synthetic construct
TDVDYYREWSWTQVS

SEQ ID NO: 124 - protein, 229 aa, synthetic construct
ALTSTEDPEPPSVPVPTNVLIKSYNLNPVVCWEYQNMSQTPIFTVQVKVYSGSWTDSCTNISDHCCNIYEQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCLSVDGISSFWQVRTEKSKDVCIPPFHDDRKDS

SEQ ID NO: 125 - protein, 313 aa, synthetic construct
ASSPDSFSQLAAPLNPRLHLYNDEQILTWEPSPSSNDPRPVVYQVEYSFIDGSWHRLLEPNCTDITETKCDLTGGGRLKLFPHPFTVFLRVRAKRGNLTSKWVGLEPFQHYENVTVGPPKNISVTPGKGSLVIHFSPPFDVFHGATFQYLVHYWEKSETQQEQVEGPFKSNSIVLGNLKPYRVYCLQTEAQLILKNKKIRPHGLLSNVSCHETTANASARLQQVILIPLGIFALLLGLTGACFTLFLKYQSRVKYWFQAPPNIPEQIEEYLKDPDQFILEVLDKDGSPKEDSWDSVSIISSPEKERDDVLQTP

SEQ ID NO: 126 - protein, 228 aa, synthetic construct [sequence omitted]

SEQ ID NO: 127 - protein, 220 aa, synthetic construct
SQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 128 - protein, 6 aa, synthetic construct
CISPRG

SEQ ID NO: 129 - protein, 7 aa, synthetic construct
CISPRGC

SEQ ID NO: 130 - protein, 8 aa, synthetic construct
CISPRGCG

SEQ ID NO: 131 - protein, 15 aa, synthetic construct
CISPRGCPDGPYVMY

SEQ ID NO: 132 - protein, 14 aa, synthetic construct
CISPRGCPDGPYVM

SEQ ID NO: 133 - protein, 15 aa, synthetic construct
CISPRGCEPGTYVPT

SEQ ID NO: 134 - protein, 15 aa, synthetic construct
CISPRGCPGQIWHPP

SEQ ID NO: 135 - protein, 15 aa, synthetic construct
GSHCLIPINMGAPSC

SEQ ID NO: 136 - protein, 32 aa, synthetic construct
CISPRGCGGSSASQSGQGSHCLIPINMGAPSC

SEQ ID NO: 137 - protein, 19 aa, synthetic construct
CNHHYFYTCGCISPRGCPG

SEQ ID NO: 138 - protein, 19 aa, synthetic construct
ADHVFWGSYGCISPRGCPG

SEQ ID NO: 139 - protein, 19 aa, synthetic construct
CHHVYWGHCGCISPRGCPG

SEQ ID NO: 140 - protein, 19 aa, synthetic construct
CPHFTTTSCGCISPRGCPG

SEQ ID NO: 141 - protein, 19 aa, synthetic construct
CNHHYHYYCGCISPRGCPG

SEQ ID NO: 142 - protein, 19 aa, synthetic construct
CPHVSFGSCGCISPRGCPG

SEQ ID NO: 143 - protein, 19 aa, synthetic construct
CPYYTLSYCGCISPRGCPG

SEQ ID NO: 144 - protein, 19 aa, synthetic construct
CNHVYFGTCGCISPRGCPG

SEQ ID NO: 145 - protein, 19 aa, synthetic construct
CNHFTLTTCGCISPRGCPG

SEQ ID NO: 146 - protein, 19 aa, synthetic construct
CHHFTLTTCGCISPRGCPG

SEQ ID NO: 147 - protein, 18 aa, synthetic construct
YNPCATPMCCISPRGCPG

SEQ ID NO: 148 - protein, 18 aa, synthetic construct
CNHHYFYTCGCISPRGCG

SEQ ID NO: 149 - protein, 18 aa, synthetic construct
CNHHYHYYCGCISPRGCG

SEQ ID NO: 150 - protein, 18 aa, synthetic construct
CNHVYFGTCGCISPRGCG

SEQ ID NO: 151 - protein, 18 aa, synthetic construct
CHHVYWGHCGCISPRGCG

SEQ ID NO: 152 - protein, 18 aa, synthetic construct
CPHFTTTSCGCISPRGCG

SEQ ID NO: 153 - protein, 18 aa, synthetic construct
CNHFTLTTCGCISPRGCG

SEQ ID NO: 154 - protein, 18 aa, synthetic construct
CHHFTLTTCGCISPRGCG

SEQ ID NO: 155 - protein, 18 aa, synthetic construct
CPYYTLSYCGCISPRGCG

SEQ ID NO: 156 - protein, 18 aa, synthetic construct
CPHVSFGSCGCISPRGCG

SEQ ID NO: 157 - protein, 18 aa, synthetic construct
ADHVFWGSYGCISPRGCG

SEQ ID NO: 158 - protein, 17 aa, synthetic construct
YNPCATPMCCISPRGCG

SEQ ID NO: 159 - protein, 18 aa, synthetic construct
CHHVYWGHCGCISPRGCG

SEQ ID NO: 160 - protein, 15 aa, synthetic construct
CISPRGCGQPIPSVK

SEQ ID NO: 161 - protein, 15 aa, synthetic construct
CISPRGCTQPYHVSR

SEQ ID NO: 162 - protein, 15 aa, synthetic construct
CISPRGCNAVSGLGS